SEQUENCE LISTING (310 sequences)

SEQ ID NO: 1 - DNA, 58 bp, synthetic construct
  source 1..58: mol_type = other DNA
ggtggaggcggtagcggaggcggagggtcggctagcgaggtgcagctggtggagtctg

SEQ ID NO: 2 - DNA, 68 bp, synthetic construct
  source 1..68: mol_type = other DNA
ttacaagtcctcttcagaaataagcttttgttcggatccgccccctaggacggtcagcttggtccctc

SEQ ID NO: 3 - protein, 4 aa, synthetic construct
GGGS

SEQ ID NO: 4 - DNA, 38 bp, synthetic construct
  source 1..38: mol_type = other DNA
acgatgacgacaaggctagcgaggtgcagctggtggag

SEQ ID NO: 5 - DNA, 38 bp, synthetic construct
  source 1..38: mol_type = other DNA
tggtgatgatgatggggcgggccccctaggacggtcag

SEQ ID NO: 6 - DNA, 32 bp, synthetic construct
  source 1..32: mol_type = other DNA
agctgaccgtcctagggggcccgccccatcat

SEQ ID NO: 7 - DNA, 32 bp, synthetic construct
  source 1..32: mol_type = other DNA
gactccaccagctgcacctcgctagccttgtc

SEQ ID NO: 8 - protein, 30 aa, synthetic construct
EVQLVESGGGLVQPGGSLRLSCAASGFTFS

SEQ ID NO: 9 - protein, 5 aa, synthetic construct
SYGMH

SEQ ID NO: 10 - protein, 14 aa, synthetic construct
WVRQAPGKGLEWVA

SEQ ID NO: 11 - protein, 17 aa, synthetic construct
VISYDGSNKYYADSVKG

SEQ ID NO: 12 - protein, 31 aa, synthetic construct
FTISRDNSKNTLYLQMNSLRAEDTAVYYCAR

SEQ ID NO: 13 - protein, 7 aa, synthetic construct
NLRHEKQ

SEQ ID NO: 14 - protein, 9 aa, synthetic construct
WGQGTLVTV

SEQ ID NO: 15 - protein, 22 aa, synthetic construct
QSVLTQPPSASGTPGQRVTISC

SEQ ID NO: 16 - protein, 13 aa, synthetic construct
SGSSSNIGSNYVY

SEQ ID NO: 17 - protein, 15 aa, synthetic construct
WYQQLPGTAPKLLIY

SEQ ID NO: 18 - protein, 7 aa, synthetic construct
RNNQRPS

SEQ ID NO: 19 - protein, 32 aa, synthetic construct
GVPDRFSGSKSGTSASLAISGLRSEDEADYYC

SEQ ID NO: 20 - protein, 9 aa, synthetic construct
AAWDDSLFV

SEQ ID NO: 21 - protein, 10 aa, synthetic construct
FGGGTKLTVL

SEQ ID NO: 22 - protein, 30 aa, synthetic construct; identical to SEQ ID NO: 8

SEQ ID NO: 23 - protein, 5 aa, synthetic construct
SYGMH

SEQ ID NO: 24 - protein, 14 aa, synthetic construct
WVRQAPGKGLEWVA

SEQ ID NO: 25 - protein, 17 aa, synthetic construct
VISYDGSNKYYADSVKG

SEQ ID NO: 26 - protein, 31 aa, synthetic construct; identical to SEQ ID NO: 12

SEQ ID NO: 27 - protein, 7 aa, synthetic construct
NLRHEKQ

SEQ ID NO: 28 - protein, 9 aa, synthetic construct
WGQGTLVTV

SEQ ID NO: 29 - protein, 22 aa, synthetic construct
QSVLTQPPSASGTPGQRVTSSC

SEQ ID NO: 30 - protein, 13 aa, synthetic construct
SGSSSNIGSNYVY

SEQ ID NO: 31 - protein, 15 aa, synthetic construct
WYRQLPGTAPKLLIY

SEQ ID NO: 32 - protein, 7 aa, synthetic construct
RNNHRPS

SEQ ID NO: 33 - protein, 32 aa, synthetic construct; identical to SEQ ID NO: 19

SEQ ID NO: 34 - protein, 9 aa, synthetic construct
AAWDDRLFV

SEQ ID NO: 35 - protein, 10 aa, synthetic construct
FGGGTKLTVL

SEQ ID NO: 36 - protein, 30 aa, synthetic construct; identical to SEQ ID NO: 8

SEQ ID NO: 37 - protein, 5 aa, synthetic construct
SYGMH

SEQ ID NO: 38 - protein, 14 aa, synthetic construct
WVRQAPGKGLERVA

SEQ ID NO: 39 - protein, 17 aa, synthetic construct
VISYDGSNKYYADSVKG

SEQ ID NO: 40 - protein, 31 aa, synthetic construct
FTISRDNSKNTLYLRMNSLRAEDTAVYYCAR

SEQ ID NO: 41 - protein, 7 aa, synthetic construct
NLRHEKQ

SEQ ID NO: 42 - protein, 9 aa, synthetic construct
WGQGTLVTV

SEQ ID NO: 43 - protein, 22 aa, synthetic construct
QSVLTQPPSASGTPGQRVTISC

SEQ ID NO: 44 - protein, 13 aa, synthetic construct
SGSSSNIGSNYVY

SEQ ID NO: 45 - protein, 15 aa, synthetic construct
WYRHLPGTAPKLLIY

SEQ ID NO: 46 - protein, 7 aa, synthetic construct
RKNQRPS

SEQ ID NO: 47 - protein, 32 aa, synthetic construct; identical to SEQ ID NO: 19

SEQ ID NO: 48 - protein, 9 aa, synthetic construct
AAWDDSLFV

SEQ ID NO: 49 - protein, 10 aa, synthetic construct
FGGGTKLTVL

SEQ ID NO: 50 - protein, 679 aa, synthetic construct
MGVLLTQRTLLSLVLALLFPSMASMDYKDDDDKASEVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLFVFGGGTKLTVLGAPPHHHHHHGAAEQKLISEEDLVKGKHLCPSPLFPGPSKPFWVLVVVGGVLACYSLLVTVAFIIFWVRSKRSRLLHSDYMNMTPRRPGPTRKHYQPYAPPRDFAAYRSQVRKAAITSYEKSDGVYTGLSTRNQETYETLKHEKPPQEGRGSLLTCGDVEENPGPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 51 - protein, 23 aa, synthetic construct
MGVLLTQRTLLSLVLALLFPSMA

SEQ ID NO: 52 - protein, 8 aa, synthetic construct
DYKDDDDK

SEQ ID NO: 53 - protein, 116 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSS

SEQ ID NO: 54 - protein, 15 aa, synthetic construct
GGGGSGGGGSGGSAL

SEQ ID NO: 55 - protein, 108 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 56 - protein, 6 aa, synthetic construct
HHHHHH

SEQ ID NO: 57 - protein, 10 aa, synthetic construct
EQKLISEEDL

SEQ ID NO: 58 - protein, 18 aa, synthetic construct
VKGKHLCPSPLFPGPSKP

SEQ ID NO: 59 - protein, 27 aa, synthetic construct
FWVLVVVGGVLACYSLLVTVAFIIFWV

SEQ ID NO: 60 - protein, 39 aa, synthetic construct
RSKRSRLLHSDYMNMTPRRPGPTRKHYQPYAPPRDFAAY

SEQ ID NO: 61 - protein, 40 aa, synthetic construct
RSQVRKAAITSYEKSDGVYTGLSTRNQETYETLKHEKPPQ

SEQ ID NO: 62 - protein, 18 aa, synthetic construct
EGRGSLLTCGDVEENPGP

SEQ ID NO: 63 - protein, 240 aa, synthetic construct
PVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 64 - DNA, 2040 bp, synthetic construct
  source 1..2040: mol_type = other DNA
atgggggtactgctcacacagaggacgctgctcagtctggtccttgcactcctgtttccaagcatggcatcgatggactacaaagacgatgacgacaaggctagcgaggtgcagctggtggagtctgggggaggcttggtacagcctggggggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaactccaagaacacgctgtatctgcaaatgaacagcctgagagctgaggacacggccgtgtattactgtgcaagaaatctgaggcatgagaagcagtggggccaaggtaccctggtcaccgtctcgagtggtggaggcggttcaggcggaggtggctctggcggtagtgcacttcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgtttgtattcggcggagggaccaagctgaccgtcctaggtgcgccgccccatcatcatcaccatcacggggccgcagaacaaaaactcatctcagaagaggatctggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcaagtgcgaaaggcagctataaccagctatgagaaatcagatggtgtttacacgggcctgagcaccaggaaccaggagacttacgagactctgaagcatgagaaaccaccacaggagggcagaggaagtcttctaacatgcggtgacgtggaggagaatcccggcccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 65 - DNA, 69 bp, synthetic construct
  source 1..69: mol_type = other DNA
atgggggtactgctcacacagaggacgctgctcagtctggtccttgcactcctgtttccaagcatggca

SEQ ID NO: 66 - DNA, 24 bp, synthetic construct
  source 1..24: mol_type = other DNA
gactacaaagacgatgacgacaag

SEQ ID NO: 67 - DNA, 348 bp, synthetic construct
  source 1..348: mol_type = other DNA
gaggtgcagctggtggagtctgggggaggcttggtacagcctggggggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaactccaagaacacgctgtatctgcaaatgaacagcctgagagctgaggacacggccgtgtattactgtgcaagaaatctgaggcatgagaagcagtggggccaaggtaccctggtcaccgtctcgagt

SEQ ID NO: 68 - DNA, 45 bp, synthetic construct
  source 1..45: mol_type = other DNA
ggtggaggcggttcaggcggaggtggctctggcggtagtgcactt

SEQ ID NO: 69 - DNA, 324 bp, synthetic construct
  source 1..324: mol_type = other DNA
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgtttgtattcggcggagggaccaagctgaccgtccta

SEQ ID NO: 70 - DNA, 18 bp, synthetic construct
  source 1..18: mol_type = other DNA
catcatcatcaccatcac

SEQ ID NO: 71 - DNA, 30 bp, synthetic construct
  source 1..30: mol_type = other DNA
gaacaaaaactcatctcagaagaggatctg

SEQ ID NO: 72 - DNA, 54 bp, synthetic construct
  source 1..54: mol_type = other DNA
gtgaaagggaaacacctttgtccaagtcccctatttcccggaccttctaagccc

SEQ ID NO: 73 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 74 - DNA, 117 bp, synthetic construct
  source 1..117: mol_type = other DNA
aggagtaagaggagcaggctcctgcacagtgactacatgaacatgactccccgccgccccgggcccacccgcaagcattaccagccctatgccccaccacgcgacttcgcagcctat

SEQ ID NO: 75 - DNA, 120 bp, synthetic construct
  source 1..120: mol_type = other DNA
agatctcaagtgcgaaaggcagctataaccagctatgagaaatcagatggtgtttacacgggcctgagcaccaggaaccaggagacttacgagactctgaagcatgagaaaccaccacag

SEQ ID NO: 76 - DNA, 54 bp, synthetic construct
  source 1..54: mol_type = other DNA
gagggcagaggaagtcttctaacatgcggtgacgtggaggagaatcccggccca

SEQ ID NO: 77 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 78 - protein, 45 aa, synthetic construct
TTTPAPRPPTPAPTIASQPLSLRPEACRPAAGGAVHTRGLDFACD

SEQ ID NO: 79 - protein, 29 aa, synthetic construct
IYIWAPLAGTCGVLLLSLVITLYCNHRNR

SEQ ID NO: 80 - protein, 42 aa, synthetic construct
KRGRKKLLYIFKQPFMRPVQTTQEEDGCSCRFPEEEEGGCEL

SEQ ID NO: 81 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 795 aa, synthetic construct
MGVLLTQRTLLSLVLALLFPSMASMDYKDDDDKASEVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLFVFGGGTKLTVLGAPPHHHHHHGAAEQKLISEEDLTSTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAIAGSGEGRGSLLTCGDVEENPGPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 83 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 84 - DNA, 87 bp, synthetic construct
  source 1..87: mol_type = other DNA
atctacatctgggcgcccttggccgggacttgtggggtccttctcctgtcactggttatcaccctttactgcaaccacaggaaccgt

SEQ ID NO: 85 - DNA, 124 bp, synthetic construct
  source 1..124: mol_type = other DNA
aaacggggcagaaagaaactcctgtatatattcaaacaaccatttatgagaccagtacaaactactcaagaggaagatggctgtagctgccgatttccagaagaagaagaaggaggatgtgaac

SEQ ID NO: 86 - DNA, 338 bp, synthetic construct
  source 1..338: mol_type = other DNA
tgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 87 - DNA, 2388 bp, synthetic construct
  source 1..2388: mol_type = other DNA
atgggggtactgctcacacagaggacgctgctcagtctggtccttgcactcctgtttccaagcatggcatcgatggactacaaagacgatgacgacaaggctagcgaggtgcagctggtggagtctgggggaggcttggtacagcctggggggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaactccaagaacacgctgtatctgcaaatgaacagcctgagagctgaggacacggccgtgtattactgtgcaagaaatctgaggcatgagaagcagtggggccaaggtaccctggtcaccgtctcgagtggtggaggcggttcaggcggaggtggctctggcggtagtgcacttcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgtttgtattcggcggagggaccaagctgaccgtcctaggtgcgccgccccatcatcatcaccatcacggggccgcagaacaaaaactcatctcagaagaggatctgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgatcgcaggcagcggcgagggcagaggaagtcttctaacatgcggtgacgtggaggagaatcccggcccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 88 - protein, 795 aa, synthetic construct
MGVLLTQRTLLSLVLALLFPSMASMDYKDDDDKASEVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTSSCSGSSSNIGSNYVYWYRQLPGTAPKLLIYRNNHRPSGVPDRFSGSKSGTSASLAISGLRSEDEADYYCAAWDDRLFVFGGGTKLTVLGGPPHHHHHHGAAEQKLISEEDLTSTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAIAGSGEGRGSLLTCGDVEENPGPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 89 - DNA, 2388 bp, synthetic construct
  source 1..2388: mol_type = other DNA
atgggggtactgctcacacagaggacgctgctcagtctggtccttgcactcctgtttccaagcatggcatcgatggactacaaagacgatgacgacaaggctagcgaggtgcagctggtggagtctgggggaggcttggtacagcctggggggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaactccaagaacacgctgtatctgcaaatgaacagcctgagagctgaggacacggccgtgtattactgtgcaagaaatctgaggcatgagaagcagtggggccaaggtaccctggtcaccgtctcgagtggtggaggcggttcaggcggaggtggctctggcggtagtgcacttcagtctgtgctgactcagccaccctcagcgtctgggacccccgggcagagggtcaccagctcttgttctggaagcagctccaacatcggaagtaattatgtatactggtaccggcagctcccaggaacggcccccaaactcctcatctataggaataatcatcggccctcaggggtccctgaccgattctctggctccaagtctggcacctcagcctccctggccatcagtgggctccggtccgaggatgaggctgattattactgtgcagcatgggatgacagactgtttgtattcggcggagggaccaagctgaccgtcctagggggcccgccccatcatcatcaccatcacggggccgcagaacaaaaactcatctcagaagaggatctgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgatcgcaggcagcggcgagggcagaggaagtcttctaacatgcggtgacgtggaggagaatcccggcccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 90 - protein, 795 aa, synthetic construct
MGVLLTQRTLLSLVLALLFPSMASMDYKDDDDKASEVQLVESGGGLVQPGGSLRLSCAASGFTFSSYGMHWVRQAPGKGLERVAVISYDGSNKYYADSVKGRFTISRDNSKNTLYLRMNSLRAEDTAVYYCARNLRHEKQWGQGTLVTVSSGGGGSGGGGSGGSALQSVLTQPPSASGTPGQRVTISCSGSSSNIGSNYVYWYRHLPGTAPKLLIYRKNQRPSGVPDRFSGSKSGTSASLAISGLRSEDEADYYCAAWDDSLFVFGGGTKLTVLGGPPHHHHHHGAAEQKLISEEDLTSTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAIAGSGEGRGSLLTCGDVEENPGPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 91 - DNA, 2388 bp, synthetic construct
  source 1..2388: mol_type = other DNA
atgggggtactgctcacacagaggacgctgctcagtctggtccttgcactcctgtttccaagcatggcatcgatggactacaaagacgatgacgacaaggctagcgaggtgcagctggtggagtctgggggaggcttggttcagcctggggggtccctgagactctcctgtgcagcctctggattcaccttcagtagctatggcatgcactgggtccgccaggctccaggcaaggggctggagcgggtggcagttatatcatatgatggaagtaataaatactatgcagactccgtgaagggccgattcaccatctccagagacaactccaagaacacgctgtatctgcgaatgaacagcctgagagctgaggacacggccgtgtattactgtgcaagaaatctgaggcatgagaagcagtggggccaaggtaccctggtcaccgtctcgagtggtggaggcggttcaggcggaggtggctctggcggtagtgcacttcagtctgtgctgactcagccaccctcagcgtctgggacccccgggcagagggtcaccatctcttgttctggaagcagctccaacatcggaagtaattatgtatactggtaccggcacctcccaggaacggcccccaaactcctcatctataggaaaaatcagcggccctcaggggtccctgaccgattctctggctccaagtctggcacctcagcctccctggccatcagtgggctccggtccgaggatgaggctgattattactgtgcagcatgggatgacagcctgtttgtattcggcggagggaccaagctgaccgtcctagggggcccgccccatcatcatcaccatcacggggccgcagaacaaaaactcatctcagaagaggatctgactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcgatcgcaggcagcggcgagggcagaggaagtcttctaacatgcggtgacgtggaggagaatcccggcccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 92 - protein, 116 aa, synthetic construct; identical to SEQ ID NO: 53

SEQ ID NO: 93 - protein, 108 aa, synthetic construct
QSVLTQPPSASGTPGQRVTSSCSGSSSNIGSNYVYWYRQLPGTAPKLLIYRNNHRPSGVPDRFSGSKSGTSASLAISGLRSEDEADYYCAAWDDRLFVFGGGTKLTVL

SEQ ID NO: 94 - DNA, 348 bp, synthetic construct; identical to SEQ ID NO: 67
  source 1..348: mol_type = other DNA

SEQ ID NO: 95 - DNA, 324 bp, synthetic construct
  source 1..324: mol_type = other DNA
cagtctgtgctgactcagccaccctcagcgtctgggacccccgggcagagggtcaccagctcttgttctggaagcagctccaacatcggaagtaattatgtatactggtaccggcagctcccaggaacggcccccaaactcctcatctataggaataatcatcggccctcaggggtccctgaccgattctctggctccaagtctggcacctcagcctccctggccatcagtgggctccggtccgaggatgaggctgattattactgtgcagcatgggatgacagactgtttgtattcggcggagggaccaagctgaccgtccta

SEQ ID NO: 96 - protein, 116 aa, synthetic construct
EVQLVESGGGLVQPGGSLRLSCAASGFTFSSYGMHWVRQAPGKGLERVAVISYDGSNKYYADSVKGRFTISRDNSKNTLYLRMNSLRAEDTAVYYCARNLRHEKQWGQGTLVTVSS

SEQ ID NO: 97 - protein, 108 aa, synthetic construct
QSVLTQPPSASGTPGQRVTISCSGSSSNIGSNYVYWYRHLPGTAPKLLIYRKNQRPSGVPDRFSGSKSGTSASLAISGLRSEDEADYYCAAWDDSLFVFGGGTKLTVL

SEQ ID NO: 98 - DNA, 348 bp, synthetic construct
  source 1..348: mol_type = other DNA
gaggtgcagctggtggagtctgggggaggcttggttcagcctggggggtccctgagactctcctgtgcagcctctggattcaccttcagtagctatggcatgcactgggtccgccaggctccaggcaaggggctggagcgggtggcagttatatcatatgatggaagtaataaatactatgcagactccgtgaagggccgattcaccatctccagagacaactccaagaacacgctgtatctgcgaatgaacagcctgagagctgaggacacggccgtgtattactgtgcaagaaatctgaggcatgagaagcagtggggccaaggtaccctggtcaccgtctcgagt

SEQ ID NO: 99 - DNA, 324 bp, synthetic construct
  source 1..324: mol_type = other DNA
cagtctgtgctgactcagccaccctcagcgtctgggacccccgggcagagggtcaccatctcttgttctggaagcagctccaacatcggaagtaattatgtatactggtaccggcacctcccaggaacggcccccaaactcctcatctataggaaaaatcagcggccctcaggggtccctgaccgattctctggctccaagtctggcacctcagcctccctggccatcagtgggctccggtccgaggatgaggctgattattactgtgcagcatgggatgacagcctgtttgtattcggcggagggaccaagctgaccgtccta

SEQ ID NO: 100 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYMQMNSLRAEDTAVYYCARNLRHEKQWGRGTLVTVSSGGGGSGGGGSGGSALQSVLTQTPSASGTPGQRVTISRSGSSSNIGSNYVYWYQQLPGTAPKLLIYRNNQRPSGVHDRFSGSKSGTSASLAISGLRSEDEADYYCAAWDDSLFVFGGGTKLTVL

SEQ ID NO: 101 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISYSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 102 - protein, 239 aa, synthetic construct
EVQLVESGGGLVQPGGSLRLSCAASGFTFSSYCMHWVRQAPGKGLEWVAVISYDGSNKYCADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNDQRPSGVPDRFSGSKSGTSASPAISGLRFEDEADYYSAARDDSLFVFGGGTKLTVL

SEQ ID NO: 103 - protein, 240 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYRAAWDDSLFVFGGGTKLTVL

SEQ ID NO: 104 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTTASLAISGLRSEDEADYYSAAWDDSLFVFGGGTKLTVL

SEQ ID NO: 105 - protein, 239 aa, synthetic construct
EVQLVESGGGLVQPRGSLRLSCAASGFTFSSFGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPRQRVTISSSGSSSNIGSNYVYWYQQLPGTAPKLLIYRNNQRPSGAPDRFSGSKSGTSASRAISGLRSEDEADYYRAAWDDSLFVFGGGTKLTVL

SEQ ID NO: 106 - protein, 239 aa, synthetic construct
EVQPVESGGGLVQPGGSLRLSCAASGFTFSSYGMHWVRQAPGKGLEWVAVISYDGSNKYYADSVKGRYTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 107 - protein, 239 aa, synthetic construct
EAQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYRAAWDDSLFVFGGGTKLTVL

SEQ ID NO: 108 - protein, 239 aa, synthetic construct
EVQLVESGGGLVQPGGSLRLSCAASGFTFSSYGMHWVRQAPGKGLERVAVISYDGSNKYYADSAKGRFAVSRDNSKNTLYLRMNCLRAEDTAVYYCARNLRHEKQWGQGTLVTVSRGGGGSGGGGSGGSALQSVLTQPPSASGTPGQRVIISCSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 109 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYCCARNLRHEKQWGQGTLVTVSSGGGGSGGGGSGGSALQSVLTQPPSASGTPGQRVTISCSGSSSNIGSNYVYWYQQHPGTAPKLLIYRYNQRPSGVPDRFSGSKSGTSASLAISGLRSEDEADYYCAAWDDSLFVFGGGTKLTVL

SEQ ID NO: 110 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTSSCSGSSSNIGSNYVYWYRQLPGTAPKLLIYRNNHRPSGVPDRFSGSKSGTSASLAISGLRSEDEADYYCAAWDDRLFVFGGGTKLTVL

SEQ ID NO: 111 - protein, 239 aa, synthetic construct
EVQLVESGGGLVQPGGSLRLSCAASGFTFSSYGMHWVRQAPGKGLERVAVISYDGSNKYYADSVKGRFTISRDNSKNTLYLRMNSLRAEDTAVYYCARNLRHEKQWGQGTLVTVSSGGGGSGGGGSGGSALQSVLTQPPSASGTPGQRVTISCSGSSSNIGSNYVYWYRHLPGTAPKLLIYRKNQRPSGVPDRFSGSKSGTSASLAISGLRSEDEADYYCAAWDDSLFVFGGGTKLTVL

SEQ ID NO: 112 - protein, 239 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCARNLRHEKQWGQGTLVTVSSGGGGSGGGGSGGSALQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 113 - protein, 717 aa, synthetic construct
GAGGTGCAGCTGGTGGAGTCTGGGGGAGGCTTGGTACAGCCTGGGGGGTCCCTGAGACTCTCCTGTGCAGCCTCTGGATTCACCTTCAGTAGCTATGGCATGCACTGGGTCCGCCAGGCTCCAGGCAAGGGGCTGGAGTGGGTGGCAGTTATATCATATGATGGAAGTAATAAATACTATGCAGACTCCGTGAAGGGCCGATTCACCATCTCCAGAGACAACTCCAAGAACACGCTGTATCTGCAAATGAACAGCCTGAGAGCTGAGGACACGGCCGTGTATTACTGTGCAAGAAATCTGAGGCATGAGAAGCAGTGGGGCCAAGGTACCCTGGTCACCGTCTCGAGTGGTGGAGGCGGTTCAGGCGGAGGTGGCTCTGGCGGTAGTGCACTTCAGTCTGTGCTGACTCAGCCACCCTCAGCGTCTGGGACCCCCGGGCAGAGGGTCACCATCTCTTGTTCTGGAAGCAGCTCCAACATCGGAAGTAATTATGTATACTGGTACCAGCAGCTCCCAGGAACGGCCCCCAAACTCCTCATCTATAGGAATAATCAGCGGCCCTCAGGGGTCCCTGACCGATTCTCTGGCTCCAAGTCTGGCACCTCAGCCTCCCTGGCCATCAGTGGGCTCCGGTCCGAGGATGAGGCTGATTATTACTGTGCAGCATGGGATGACAGCCTGTTTGTATTCGGCGGAGGGACCAAGCTGACCGTCCTA

SEQ ID NO: 114 - protein, 717 aa, synthetic construct
GAGGTGCAGCTGGTGGAGTCTGGGGGAGGCTTGGTACAGCCTGGGGGGTCCCTGAGACTCTCCTGTGCAGCCTCTGGATTCACCTTCAGTAGCTATGGCATGCACTGGGTCCGCCAGGCTCCAGGCAAGGGGCTGGAGTGGGTGGCAGTTATATCATATGATGGAAGTAATAAATACTATGCAGACTCCGTGAAGGGCCGATTCACCATCTCCAGAGACAACTCCAAGAACACGCTGTATCTGCAAATGAACAGCCTGAGAGCTGAGGACACGGCCGTGTATTACTGTGCAAGAAATCTGAGGCATGAGAAGCAGTGGGGCCAAGGTACCCTGGTCACCGTCTCGAGTGGTGGAGGCGGTTCAGGCGGAGGTGGCTCTGGCGGTAGTGCACTTCAGTCTGTGCTGACTCAGCCACCCTCAGCGTCTGGGACCCCCGGGCAGAGGGTCACCAGCTCTTGTTCTGGAAGCAGCTCCAACATCGGAAGTAATTATGTATACTGGTACCGGCAGCTCCCAGGAACGGCCCCCAAACTCCTCATCTATAGGAATAATCATCGGCCCTCAGGGGTCCCTGACCGATTCTCTGGCTCCAAGTCTGGCACCTCAGCCTCCCTGGCCATCAGTGGGCTCCGGTCCGAGGATGAGGCTGATTATTACTGTGCAGCATGGGATGACAGACTGTTTGTATTCGGCGGAGGGACCAAGCTGACCGTCCTA

SEQ ID NO: 115 - protein, 717 aa, synthetic construct
GAGGTGCAGCTGGTGGAGTCTGGGGGAGGCTTGGTTCAGCCTGGGGGGTCCCTGAGACTCTCCTGTGCAGCCTCTGGATTCACCTTCAGTAGCTATGGCATGCACTGGGTCCGCCAGGCTCCAGGCAAGGGGCTGGAGCGGGTGGCAGTTATATCATATGATGGAAGTAATAAATACTATGCAGACTCCGTGAAGGGCCGATTCACCATCTCCAGAGACAACTCCAAGAACACGCTGTATCTGCGAATGAACAGCCTGAGAGCTGAGGACACGGCCGTGTATTACTGTGCAAGAAATCTGAGGCATGAGAAGCAGTGGGGCCAAGGTACCCTGGTCACCGTCTCGAGTGGTGGAGGCGGTTCAGGCGGAGGTGGCTCTGGCGGTAGTGCACTTCAGTCTGTGCTGACTCAGCCACCCTCAGCGTCTGGGACCCCCGGGCAGAGGGTCACCATCTCTTGTTCTGGAAGCAGCTCCAACATCGGAAGTAATTATGTATACTGGTACCGGCACCTCCCAGGAACGGCCCCCAAACTCCTCATCTATAGGAAAAATCAGCGGCCCTCAGGGGTCCCTGACCGATTCTCTGGCTCCAAGTCTGGCACCTCAGCCTCCCTGGCCATCAGTGGGCTCCGGTCCGAGGATGAGGCTGATTATTACTGTGCAGCATGGGATGACAGCCTGTTTGTATTCGGCGGAGGGACCAAGCTGACCGTCCTA

SEQ ID NO: 116 - protein, 30 aa, synthetic construct; identical to SEQ ID NO: 8

SEQ ID NO: 117 - protein, 5 aa, synthetic construct
SYGMH

SEQ ID NO: 118 - protein, 14 aa, synthetic construct
WVRQAPGKGLEWVA

SEQ ID NO: 119 - protein, 17 aa, synthetic construct
VISYDGSNKYYADSVKG

SEQ ID NO: 120 - protein, 32 aa, synthetic construct
RFTISRDNSKNTLYMQMNSLRAEDTAVYYCAR

SEQ ID NO: 121 - protein, 7 aa, synthetic construct
NLRHEKQ

SEQ ID NO: 122 - protein, 11 aa, synthetic construct
WGRGTLVTVSS

SEQ ID NO: 123 - protein, 22 aa, synthetic construct
QSVLTQTPSASGTPGQRVTISR

SEQ ID NO: 124 - protein, 13 aa, synthetic construct
SGSSSNIGSNYVY

SEQ ID NO: 125 - protein, 15 aa, synthetic construct
WYQQLPGTAPKLLIY

SEQ ID NO: 126 - protein, 7 aa, synthetic construct
RNNQRPS

SEQ ID NO: 127 - protein, 32 aa, synthetic construct
GVHDRFSGSKSGTSASLAISGLRSEDEADYYC

SEQ ID NO: 128 - protein, 9 aa, synthetic construct
AAWDDSLFV

SEQ ID NO: 129 - protein, 10 aa, synthetic construct
FGGGTKLTVL

SEQ ID NO: 130 - protein, 30 aa, synthetic construct; identical to SEQ ID NO: 8

SEQ ID NO: 131 - protein, 30 aa, synthetic construct; identical to SEQ ID NO: 8

SEQ ID NO: 132 - protein, 5 aa, synthetic construct
SYGMH

SEQ ID NO: 133 - protein, 14 aa, synthetic construct
WVRQAPGKGLEWVA

SEQ ID NO: 134 - protein, 17 aa, synthetic construct
VISYDGSNKYYADSVKG

SEQ ID NO: 135 - protein, 32 aa, synthetic construct
RFTISRDNSKNTLYLQMNSLRAEDTAVYYCAR

SEQ ID NO: 136 - protein, 7 aa, synthetic construct
NLRHEKQ

SEQ ID NO: 137 - protein, 11 aa, synthetic construct
WGQGTLVTVSS

SEQ ID NO: 138 - protein, 22 aa, synthetic construct
QSVLTQPPSASGTPGQRVTISY

SEQ ID NO: 139 - protein, 13 aa, synthetic construct
SGSSSNIGSNYVY

SEQ ID NO: 140 - protein, 15 aa, synthetic construct
WYQQLPGTAPKLLIY

SEQ ID NO: 141 - protein, 7 aa, synthetic construct
RNNQRPS

SEQ ID NO: 142 - protein, 32 aa, synthetic construct; identical to SEQ ID NO: 19

SEQ ID NO: 143 - protein, 9 aa, synthetic construct
AAWDDSLFV

SEQ ID NO: 144 - protein, 10 aa, synthetic construct
FGGGTKLTVL

SEQ ID NO: 145 - protein, 5 aa, synthetic construct
SYGMH

SEQ ID NO: 146 - protein, 30 aa, synthetic construct; identical to SEQ ID NO: 8

SEQ ID NO: 147 - protein, 5 aa, synthetic construct
SYCMH

SEQ ID NO: 148 - protein, 14 aa, synthetic construct
WVRQAPGKGLEWVA

SEQ ID NO: 149 - protein, 17 aa, synthetic construct
VISYDGSNKYCADSVKG

SEQ ID NO: 150 - protein, 32 aa, synthetic construct; identical to SEQ ID NO: 135

SEQ ID NO: 151 - protein, 7 aa, synthetic construct
NLRHEKQ

SEQ ID NO: 152 - protein, 11 aa, synthetic construct
WGQGTLVTVSS

SEQ ID NO: 153 - protein, 22 aa, synthetic construct
QSVLTQPPSASGTPGQRVTISC

SEQ ID NO: 154 - protein, 13 aa, synthetic construct
SGSSSNIGSNYVY

SEQ ID NO: 155 - protein, 15 aa, synthetic construct
WYQQLPGTAPKLLIY

SEQ ID NO: 156 - protein, 7 aa, synthetic construct
RNDQRPS

SEQ ID NO: 157 - protein, 32 aa, synthetic construct
GVPDRFSGSKSGTSASPAISGLRFEDEADYYS

SEQ ID NO: 158 - protein, 9 aa, synthetic construct
AARDDSLFV

SEQ ID NO: 159 - protein, 10 aa, synthetic construct
FGGGTKLTVL

SEQ ID NO: 160 - protein, 14 aa, synthetic construct
WVRQAPGKGLEWVA

SEQ ID NO: 161 - protein, 17 aa, synthetic construct
VISYDGSNKYYADSVKG

SEQ ID NO: 162 - protein, 32 aa, synthetic construct; identical to SEQ ID NO: 135

SEQ ID NO: 163 - protein, 7 aa, synthetic construct
NLRHEKQ

SEQ ID NO: 164 - protein, 11 aa, synthetic construct
WGQGTLVTVSS

SEQ ID NO: 165 - protein, 22 aa, synthetic construct
QSVLTQPPSASGTPGQRVTISC

SEQ ID NO: 166 - protein, 13 aa, synthetic construct
SGSSSNIGSNYVY

SEQ ID NO: 167 - protein, 15 aa, synthetic construct
WYQQLPGTAPKLLIY

SEQ ID NO: 168 - protein, 7 aa, synthetic construct
RNNQRPS

SEQ ID NO: 169 - protein, 32 aa, synthetic construct
GVPDRFSGSKSGTSASLAISGLRSEDEADYYR

SEQ ID NO: 170 - protein, 9 aa, synthetic construct
AAWDDSLFV

SEQ ID NO: 171 - protein, 10 aa, synthetic construct
FGGGTKLTVL

SEQ ID NO: 172 - protein, 30 aa, synthetic construct; identical to SEQ ID NO: 8

SEQ ID NO: 173 - protein, 5 aa, synthetic construct
SYGMH

SEQ ID NO: 174 - protein, 14 aa, synthetic construct
WVRQAPGKGLEWVA

SEQ ID NO: 175 - protein, 17 aa, synthetic construct
VISYDGSNKYYADSVKG

SEQ ID NO: 176 - protein, 32 aa, synthetic construct; identical to SEQ ID NO: 135

SEQ ID NO: 177 - protein, 7 aa, synthetic construct
NLRHEKQ

SEQ ID NO: 178 - protein, 11 aa, synthetic construct
WGQGTLVTVSS

SEQ ID NO: 179 - protein, 22 aa, synthetic construct
QSVLTQPPSASGTPGQRVTISC

SEQ ID NO: 180 - protein, 13 aa, synthetic construct
SGSSSNIGSNYVY

SEQ ID NO: 181 - protein, 15 aa, synthetic construct
WYQQLPGTAPKLLIY

SEQ ID NO: 182 - protein, 7 aa, synthetic construct
RNNQRPS

SEQ ID NO: 183 - protein, 32 aa, synthetic construct
GVPDRFSGSKSGTTASLAISGLRSEDEADYYS

SEQ ID NO: 184 - protein, 9 aa, synthetic construct
AAWDDSLFV

SEQ ID NO: 185 - protein, 10 aa, synthetic construct
FGGGTKLTVL

SEQ ID NO: 186 - protein, 30 aa, synthetic construct
EVQLVESGGGLVQPRGSLRLSCAASGFTFS

SEQ ID NO: 187 - protein, 5 aa, synthetic construct
SFGMH

SEQ ID NO: 188 - protein, 14 aa, synthetic construct
WVRQAPGKGLEWVA

SEQ ID NO: 189 - protein, 17 aa, synthetic construct
VISYDGSNKYYADSVKG

SEQ ID NO: 190 - protein, 32 aa, synthetic construct; identical to SEQ ID NO: 135

SEQ ID NO: 191 - protein, 7 aa, synthetic construct
NLRHEKQ

SEQ ID NO: 192 - protein, 11 aa, synthetic construct
WGQGTLVTVSS

SEQ ID NO: 193 - protein, 22 aa, synthetic construct
QSVLTQPPSASGTPRQRVTISS

SEQ ID NO: 194 - protein, 13 aa, synthetic construct
SGSSSNIGSNYVY

SEQ ID NO: 195 - protein, 15 aa, synthetic construct
WYQQLPGTAPKLLIY

SEQ ID NO: 196 - protein, 7 aa, synthetic construct
RNNQRPS

SEQ ID NO: 197 - protein, 32 aa, synthetic construct
GAPDRFSGSKSGTSASRAISGLRSEDEADYYR

SEQ ID NO: 198 - protein, 9 aa, synthetic construct
AAWDDSLFV

SEQ ID NO: 199 - protein, 10 aa, synthetic construct
FGGGTKLTVL

SEQ ID NO: 200 - protein, 30 aa, synthetic construct
EAQLVESGGGLVQPGGSLRLSCAASGFTFS

SEQ ID NO: 201 - protein, 5 aa, synthetic construct
SYGMH

SEQ ID NO: 202 - protein, 14 aa, synthetic construct
WVRQAPGKGLEWVA

SEQ ID NO: 203 - protein, 17 aa, synthetic construct
VISYDGSNKYYADSVKG

SEQ ID NO: 204 - protein, 32 aa, synthetic construct; identical to SEQ ID NO: 135

SEQ ID NO: 205 - protein, 7 aa, synthetic construct
NLRHEKQ

SEQ ID NO: 206 - protein, 11 aa, synthetic construct
WGQGTLVTVSS

SEQ ID NO: 207 - protein, 22 aa, synthetic construct
QSVLTQPPSASGTPGQRVTISC

SEQ ID NO: 208 - protein, 13 aa, synthetic construct
SGSSSNIGSNYVY

SEQ ID NO: 209 - protein, 15 aa, synthetic construct
WYQQLPGTAPKLLIY

SEQ ID NO: 210 - protein, 7 aa, synthetic construct
RNNQRPS

SEQ ID NO: 211 - protein, 32 aa, synthetic construct; identical to SEQ ID NO: 169

SEQ ID NO: 212 - protein, 9 aa, synthetic construct
AAWDDSLFV

SEQ ID NO: 213 - protein, 10 aa, synthetic construct
FGGGTKLTVL

SEQ ID NO: 214 - protein, 30 aa, synthetic construct; identical to SEQ ID NO: 8

SEQ ID NO: 215 - protein, 5 aa, synthetic construct
SYGMH

SEQ ID NO: 216 - protein, 14 aa, synthetic construct
WVRQAPGKGLERVA

SEQ ID NO: 217 - protein, 17 aa, synthetic construct
VISYDGSNKYYADSAKG

SEQ ID NO: 218 - protein, 32 aa, synthetic construct
RFAVSRDNSKNTLYLRMNCLRAEDTAVYYCAR

SEQ ID NO: 219 - protein, 7 aa, synthetic construct
NLRHEKQ

SEQ ID NO: 220 - protein, 11 aa, synthetic construct
WGQGTLVTVSR

SEQ ID NO: 221 - protein, 22 aa, synthetic construct
QSVLTQPPSASGTPGQRVIISC

SEQ ID NO: 222 - protein, 13 aa, synthetic construct
SGSSSNIGSNYVY

SEQ ID NO: 223 - protein, 15 aa, synthetic construct
WYQQLPGTAPKLLIY

SEQ ID NO: 224 - protein, 7 aa, synthetic construct
RNNQRPS

SEQ ID NO: 225 - protein, 32 aa, synthetic construct; identical to SEQ ID NO: 19

SEQ ID NO: 226 - protein, 9 aa, synthetic construct
AAWDDSLFV

SEQ ID NO: 227 - protein, 10 aa, synthetic construct
FGGGTKLTVL

SEQ ID NO: 228 - protein, 30 aa, synthetic construct
EVQPVESGGGLVQPGGSLRLSCAASGFTFS

SEQ ID NO: 229 - protein, 5 aa, synthetic construct
SYGMH

SEQ ID NO: 230 - protein, 14 aa, synthetic construct
WVRQAPGKGLEWVA

SEQ ID NO: 231 - protein, 17 aa, synthetic construct
VISYDGSNKYYADSVKG

SEQ ID NO: 232 - protein, 32 aa, synthetic construct
RYTISRDNSKNTLYLQMNSLRAEDTAVYYCAR

SEQ ID NO: 233 - protein, 7 aa, synthetic construct
NLRHEKQ

SEQ ID NO: 234 - protein, 11 aa, synthetic construct
WGQGTLVTVSS

SEQ ID NO: 235 - protein, 22 aa, synthetic construct
QSVLTQPPSASGTPGQRVTISC

SEQ ID NO: 236 - protein, 13 aa, synthetic construct
SGSSSNIGSNYVY

SEQ ID NO: 237 - protein, 15 aa, synthetic construct
WYQQLPGTAPKLLIY

SEQ ID NO: 238 - protein, 7 aa, synthetic construct
RNNQRPS

SEQ ID NO: 239 - protein, 32 aa, synthetic construct; identical to SEQ ID NO: 19

SEQ ID NO: 240 - protein, 9 aa, synthetic construct
AAWDDSLFV

SEQ ID NO: 241 - protein, 10 aa, synthetic construct
FGGGTKLTVL

SEQ ID NO: 242 - protein, 30 aa, synthetic construct; identical to SEQ ID NO: 8

SEQ ID NO: 243 - protein, 5 aa, synthetic construct
SYGMH

SEQ ID NO: 244 - protein, 14 aa, synthetic construct
WVRQAPGKGLEWVA

SEQ ID NO: 245 - protein, 17 aa, synthetic construct
VISYDGSNKYYADSVKG

SEQ ID NO: 246 - protein, 32 aa, synthetic construct
RFTISRDNSKNTLYLQMNSLRAEDTAVYCCAR

SEQ ID NO: 247 - protein, 7 aa, synthetic construct
NLRHEKQ

SEQ ID NO: 248 - protein, 11 aa, synthetic construct
WGQGTLVTVSS

SEQ ID NO: 249 - protein, 22 aa, synthetic construct
QSVLTQPPSASGTPGQRVTISC

SEQ ID NO: 250 - protein, 13 aa, synthetic construct
SGSSSNIGSNYVY

SEQ ID NO: 251 - protein, 15 aa, synthetic construct
WYQQHPGTAPKLLIY

SEQ ID NO: 252 - protein, 7 aa, synthetic construct
RNNQRPS

SEQ ID NO: 253 - protein, 32 aa, synthetic construct; identical to SEQ ID NO: 19

SEQ ID NO: 254 - protein, 9 aa, synthetic construct
AAWDDSLFV

SEQ ID NO: 255 - protein, 10 aa, synthetic construct
FGGGTKLTVL

SEQ ID NO: 256 - protein, 116 aa, synthetic construct
EVQLVESGGGLVQPGGSLRLSCAASGFTFSSYGMHWVRQAPGKGLEWVAVISYDGSNKYYADSVKGRFTISRDNSKNTLYMQMNSLRAEDTAVYYCARNLRHEKQWGRGTLVTVSS

SEQ ID NO: 257 - protein, 108 aa, synthetic construct
QSVLTQTPSASGTPGQRVTISRSGSSSNIGSNYVYWYQQLPGTAPKLLIYRNNQRPSGVHDRFSGSKSGTSASLAISGLRSEDEADYYCAAWDDSLFVFGGGTKLTVL

SEQ ID NO: 258 - protein, 116 aa, synthetic construct; identical to SEQ ID NO: 53

SEQ ID NO: 259 - protein, 108 aa, synthetic construct
QSVLTQPPSASGTPGQRVTISYSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 260 - protein, 116 aa, synthetic construct
EVQLVESGGGLVQPGGSLRLSCAASGFTFSSYCMHWVRQAPGKGLEWVAVISYDGSNKYCADSVKGRFTISRDNSKNTLYLQMNSLRAEDTAVYYCARNLRHEKQWGQGTLVTVSS

SEQ ID NO: 261 - protein, 108 aa, synthetic construct
QSVLTQPPSASGTPGQRVTISCSGSSSNIGSNYVYWYQQLPGTAPKLLIYRNDQRPSGVPDRFSGSKSGTSASPAISGLRFEDEADYYSAARDDSLFVFGGGTKLTVL

SEQ ID NO: 262 - protein, 116 aa, synthetic construct; identical to SEQ ID NO: 53

SEQ ID NO: 263 - protein, 108 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYRAAWDDSLFVFGGGTKLTVL

SEQ ID NO: 264 - protein, 116 aa, synthetic construct; identical to SEQ ID NO: 53

SEQ ID NO: 265 - protein, 108 aa, synthetic construct
QSVLTQPPSASGTPGQRVTISCSGSSSNIGSNYVYWYQQLPGTAPKLLIYRNNQRPSGVPDRFSGSKSGTTASLAISGLRSEDEADYYSAAWDDSLFVFGGGTKLTVL

SEQ ID NO: 266 - protein, 115 aa, synthetic construct
EVQLVESGGGLVQPRGSLRLSCAASGFTFSFGMHWVRQAPGKGLEWVAVISYDGSNKYYADSVKGRFTISRDNSKNTLYLQMNSLRAEDTAVYYCARNLRHEKQWGQGTLVTVSS

SEQ ID NO: 267 - protein, 108 aa, synthetic construct
QSVLTQPPSASGTPRQRVTISSSGSSSNIGSNYVYWYQQLPGTAPKLLIYRNNQRPSGAPDRFSGSKSGTSASRAISGLRSEDEADYYRAAWDDSLFVFGGGTKLTVL

SEQ ID NO: 268 - protein, 116 aa, synthetic construct
EVQPVESGGGLVQPGGSLRLSCAASGFTFSSYGMHWVRQAPGKGLEWVAVISYDGSNKYYADSVKGRYTISRDNSKNTLYLQMNSLRAEDTAVYYCARNLRHEKQWGQGTLVTVSS

SEQ ID NO: 269 - protein, 108 aa, synthetic construct; identical to SEQ ID NO: 55

SEQ ID NO: 270 - protein, 115 aa, synthetic construct
EAQLVESGGGLVQPGGSLRLSCAASGFTFSSYGMHWVRQAPGKGLEWVAVISYDGSNKYYADSVKRFTISRDNSKNTLYLQMNSLRAEDTAVYYCARNLRHEKQWGQGTLVTVSS

SEQ ID NO: 271 - protein, 108 aa, synthetic construct; identical to SEQ ID NO: 263

SEQ ID NO: 272 - protein, 116 aa, synthetic construct
EVQLVESGGGLVQPGGSLRLSCAASGFTFSSYGMHWVRQAPGKGLERVAVISYDGSNKYYADSAKGRFAVSRDNSKNTLYLRMNCLRAEDTAVYYCARNLRHEKQWGQGTLVTVSR

SEQ ID NO: 273 - protein, 108 aa, synthetic construct
QSVLTQPPSASGTPGQRVIISCSGSSSNIGSNYVYWYQQLPGTAPKLLIYRNNQRPSGVPDRFSGSKSGTSASLAISGLRSEDEADYYCAAWDDSLFVFGGGTKLTVL

SEQ ID NO: 274 - protein, 116 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYCCARNLRHEKQWGQGTLVTVSS

SEQ ID NO: 275 - protein, 108 aa, synthetic construct
QSVLTQPPSASGTPGQRVTISCSGSSSNIGSNYVYWYQQHPGTAPKLLIYRNNQRPSGVPDRFSGSKSGTSASLAISGLRSEDEADYYCAAWDDSLFVFGGGTKLTVL

SEQ ID NO: 276 - protein, 216 aa, Pseudomonas aeruginosa
AEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 277 - DNA, 651 bp, Pseudomonas aeruginosa
  source 1..651: mol_type = genomic DNA
gcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggaccttgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccacgcaacgtcggcggcgacctcgccccgtccagcatccccgaccaggaacaggcgatcagcgccctgccggactacgccagccagcccggcaaaccgtcgcgcgaggacctgaagtaa

SEQ ID NO: 278 - protein, 536 aa, Corynebacterium diphtheriae
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 279 - DNA, 1611 bp, Corynebacterium diphtheriae
  source 1..1611: mol_type = genomic DNA
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcatctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcatcggattccataggcgttcttgggtaccagaaaacagtagatcacaccaaggttaattctaagctatcgctattttttgaaatcaaaagctga

SEQ ID NO: 280 - protein, 127 aa, synthetic construct
TKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 281 - DNA, 698 bp, synthetic construct
  source 1..698: mol_type = other DNA
gggngggggacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaaaaaaaaaaaaaccccccccc

SEQ ID NO: 282 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 283 - DNA, 1311 bp, Homo sapiens
  source 1..1311: mol_type = genomic DNA
gtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaaccccccctcccactgtaggccactggatggtcatttggcatctccgtatatgtgctctggctcctcagctgagagagaaaaaaataaactgtatttggctgc

SEQ ID NO: 284 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 285 - DNA, 2406 bp, Homo sapiens
  source 1..2406: mol_type = genomic DNA
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 286 - protein, 153 aa, Homo sapiens [sequence omitted]

SEQ ID NO: 287 - DNA, 921 bp, Homo sapiens
  source 1..921: mol_type = genomic DNA
ttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 288 - protein, 365 aa, Homo sapiens [sequence omitted]

SEQ ID NO: 289 - DNA, 4000 bp, Homo sapiens
  source 1..4000: mol_type = genomic DNA
aagcttactctctggcaccaaactccatgggatgatttttccttcctagaagagtccaggtggacaggtaaggagtgggagtcagggagtccagttccagggacagagattacgggataaaaagtgaaaggagagggacggggcccatgccgagggtttctcccttgtttctcagacagctcttgggccaagactcagggagacattgagacagagcgcttggcacagaagcagaggggtcagggcgaagtccagggccccaggcgttggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggtgagtgcggggtcgggagggaaacggcctctgtggggagaagcaacgggccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaatttccacggaataggagattatcccaggtgcctgtgtccaggctggtgtctgggttctgtgctcccttccccatcccaggtgtcctgtccattctcaagatagccacatgtgtgctggaggagtgtcccatgacagatcgaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcttttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttgg

SEQ ID NO: 290 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 291 - DNA, 1601 bp, Homo sapiens
  source 1..1601: mol_type = genomic DNA
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 292 - protein, 576 aa, Ricinus communis
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 293 - DNA, 12150 bp, Ricinus communis
  source 1..12150: mol_type = genomic DNA
atatttcacgaactgatacaatatagcaagagattgaaaaaactcaaattcttacaaaactgaactgaaataaaacaagagatgcataataaaaaaatacaaatcttgatacttattacaactggaatatgcgactaagctgaaataaactgacattaaagacatgacagaaatacatgttttggtctataacaacctaagtagaaatcgcagcctgttgatgcacagatacatgtattcttatatatttgtattaatattttaatttatgtacatatcgagatgaatgaaaataagctaatatttgtcatttagaaattattccaaaactgaatcatcttacttgagttatttaattaataaaattaaatatatttcattaaacaatgcattttctctttaaaacaatccatttgaatcttactttataggaagaccttgatagataaacaatgtattggatatacgttttttttagatgctctaaagattgcattagaatgaaataaaactttttacttttaagatttttgctctttaaaataaaaagtacaaactttgataagttttatataacctaaaaagaaattgtaacatgtgagtttgagatgtcttatataatctacaaagatatttaaagagtataaacagtaaagcaataatacttaggtgatgaaaatacctcacttagtactaatacaacaaagcaagatatcgaacaaatcttaaatctttgcaaaactgaactgaagtaaaacaagaaatgcataataataagacaaatcttggaacttattatagctggaatatgccagtaagctgaaataaattgatactaaggacacaacagaaacacatgcgtctgcaacctaagaagaagttgcaacctgtcgatgcataaatatacatatgcgacgtatattgctatgtatgaaaataagcgaatacttatcatttggaatttattccaaaattggatcatcttgctgagttattcaattaataataatgaatatattttattaagaaataaaaaatttaccttttcctctttaaaacgatttatttgaatattactttatgagaagactttttataatcagaaaacttgatatataaagaatgtatttgataatgtttctttagagatgctctaaagattgtattgcacttaaataaaaccttttactcttaatagtttagtatttctcaaaattattactctttaaagtaaatagctcaaactttgagaagtcttatgtaaactaaacaaaaattgtaacctacgagtttgagatgtcttatacaacctaaaataaaatttaaagaatataaactttaagatgtctttttctatataatcaattttattttgaaaaaccaccaaagcaaaaaaaatttaggtgatggaaatatcctacctagaactaataaaatatagcaaaagattaaacaaatctcaaattcttataaaattgaactgaagtaaaacaagagatgtataataacaaattttggtacttaatacaaaaatatgtcattaagctaaagtaaagtgataataaggacacaatataaacacatgctttggcctccagcaatctaataagaaattgcaacctatcgatgcacaaatacacgtatctttatatgcttatataagtattttaatttgtgtacgtgtattcaaatgactgaaaataaactaaaacttgtcatataaaattttttcaaaattaaatgatcttgctaagttattcactaataataataaatatatttcattaaaaataaaaatttgtcctttctctataaaagagtccattagaatcttactatatatgaaggctttttataattaaaaaacttgatatataaagaatgtattggaggtgcattttttttttgagatgttctgaagattgcattggactacaataaaagtttttacttttacttcttagaattcttgctctttaaaataataagcacaaactttgagatgtcttgtagaacctaagaaaaaaattacaatttgtgatttttagatgttttatacaatctaaaaagagatttgaagagtataaattttaagatgttttatacaacctaaaacaaattgtaatctgtcgacgcacagacacgtatttttatatatttataaagatcattttaatttatgtacttatatttagataaataaaaataagctaatatttgtcatatataaattatttcataactgaattgtctgatttgttagtgttattaaattaataataatagttatattttattaaataataaaagatttaccttttctctataaaaaggggaagaaatgtggttactaaaaccatctcatattcctccgctttcttcctcagctgctcactttgtaagtattacgactttctcaaacttcctacttgttttcaattaatgttattctacctgacttcatatatatctcttttcttttgggatcattattactgctactgtaatgatcgagaaaaatctcttcttcttatcattatgacttcatatattatcatcttctatcacttcatataatgtttttgattttatgattcaatatatttataattaagttctatctttaaacgagagcaaccaattaaagaaacaagagtaaatatatataaattgtaaggtattatttgtttgatttaaatcattaaatagctagatcttctttttcttttctttttttctttctcatccaaagtttttatgaattgcaggctattagtaatatagatggtagaaagaaaattaaaattaaaacttcttcaatcatcacaaatgagagtaccaactaaactatgtgattttggtaaaaaatgcaaaacaagtacttatatatatatatatatatatatatatatatatatatatatatatattcttttataaatcagtatatacattttactctgattaccataatattatagattttactaaggtgacactaatgttatatattttggttagaatggtagtgttttcttttcttaaagatgctctagaggattcttaacaaaagaatataatatataaataatatattaaagatgccctagaaaatgcatttactgtacttaaataacctgttttgctcttaatattttattatttttatttctataataaaaaatattttaagaaatatttaagtataaaaaataaagtattttattgatgtccactgtactttttatattttatttcttattttacttttgtttccaagggcatcaatatcttcttcttttttctgtttaatttttattaataaaaaaaataattacaaatattaattaatcaaatacatagaaatttatttttataaaaaaaatccttcaaattcttttaaaatgtcattttgaccctaaatttcttttaatagttagtgttctaataaaaaaaatttacccaataatttttctaatatttcattattcttttataagacaaactcttagcctctagaattattttaaggatatatataatttgtctctctttctctttaacatagccttagtttccaataaataaataatgaaatatatttcactcttcatttctttaaacttcttacatttttttttgtagcattctttgtaagtggaatgacaaaaccgttaatgatgttcttttaaaagtgaaagatgtttatatattgcagtacagataatgatatatctactgcactacataaaacaatttaaatctccctgtttattttaagaagttatattttctttctttctcatcctaagaaagttaaattactgtaatcgacattatatgaattttaactaattccgtttctaatttataattatttcgttaaaccaatcaattccctttaaacactgcttatgcatattctgtctcaatttatatatggcatgcatcttccgtattaatttataagttcatttttattgatcaagtatttgtggttttctttatataaaaaaatgtattagtgtttttctgtattaattttataagttcatctttatgagaatgctaatgtatttggacagccaataaaattccaagaattgctgcaatcaaagatgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgccatgaaaatagatggcttaaataaaaaggacattgtaaattttgtaactgaaaggacagcaagttattgcagtccagtatctaataagagcacaactattgtcttgtgcattctaaatttatggatgaattgtatgaattaagctaattattttggtcatcagacttgatatctttttgaataaaataaataatatgttttttcaaacttataaaactatgaatgatatgaatataatgcggagactagtcaatcttttatgtaattctatgatgataaaagcttgtctcttaacttagtgaatttgtatccaagtaaaaaacagcctactaagtcatggattccttcaaatttacgctcttattataagcttaattttcatccacgatcatccctattcatgtgatgcacaagaacttaaggtatatagatttgaagtaattccttaattataatttttaagtttatcactttctttactttctaatttctttttctcaaattgtactaattaactcattgaagaatttaacaacttgttcatcagttccatagtaattctcaataaatttatggtctcacaactcaagcatccatgggttcataagttgtgataatcaggcagcttcatatatattgaaattaacataaggagtaaagtgggttgagcaatgccaggtggtacatccaggtggtacatccatatatacttcttcctccaaatcaccatgtaaaaaagtattttttacatcaaattgtcttaatgcccatcctttagttgctgcaatagaaatcagtattcaaatcgtaccaagtttcgccacaggtgcaaacgtttcctggtagtcaattccatacttctgagtaaatcctttcgctaccagtctcgcttttcgcctgtctagactcccatctgccttaagcttcactgtatagatccatttacagccaatcgtattcttcccctctggtaatttcacaaactcccatgttgcattcttctctagggctttcacctcttcctccatcgcctcttttcatttcaagtcttgcattgcctcctgcacactattaggaatcgcaatcccggataattcactagtgctaagtgcataataatctgttaaatgattatgtcgaataccattcttagttaaaaaattacttaaatgactatccataaattcctttgcattatcagtttgccaaactaaaacccatgtacgatattgatttctaataaaatttcaaaaatcacaaaaagctttacttacctcacttttgttccgaagtaaggttatccaagtcatacgagtacattcgtctataaaagttccgaaatatctagcacctgataatgaaggaatttttgcaggaccccaaacatcagaatgtataacagcaaatggttcagtacttttattaaaactaggcaaataagtagcacggtggctcttaccaagttcacacacatcacaattaaacacggaatcatccaaattaacaaacaattcaggttttaattttttcaaataactaaatgacaaatgtccaaggcgtcgatgccatagccaaattctttccattgtattacttaagctttcagtcgtgtaaacctcacttattttcttctctcctatctcagtcaagtctagataataaagtttgcctcgtttaacaccataaccaagagtctcccgagtcaggatgtcctgaaaaacacaatatgaaggccagaaagttacagtacagttaagagacgaggttatttgtccaaccgagagtaagttacaagacagtgtaagaacaatcaatacagattttaaattcagattttttgaaagagtaatggatccttctccactaacaatacatttggatccatttgttattgacacggtaaaatgagaggatggaatgaaagaataaattcgatcagaattgcataccatatgatcagatgcaccagggtctattgtccacaatgttcacacagcaagttacatttttttccagctccatttttcctgttttttccataattctggcaaacctgaaatacagtaggctccacagttgcatttgttcctcccattgtcttacattgttgatcttctcttcgaacataagcatatgtttgttctaaatcaagtttagggtctttgcgcaaaatctcacctcggacttgatcaaagtttgaatcaagcccactgaggaagatatgcatgcgtagccttgccatggcagaatgcaacgctactactccatccactgtaccttcatgtgactgagtacgatgatctatctcttgaaagatttccatcaactcagagtaatacgtaggcaatggtctgccctcttgtttgattgcgaaagacttctgattcaattcgaataacctcgtttcatcagttccatcataaaatgtttttgctgctgcctcccaaacatctttggcagtaggaagtctgatgaaatgttgcatcagtgaaggtatcatcgaatcaatcagccagctctttaccttttgattctcagttatccaggtagagtatctcgtatctgtagttgcaggcttcacttccgatccggttaaatacccagttttatttcgtgctccaatacgcatctccatgagctgtgaccatagagaataattcgattcatccagtataacactggtagggaatgatacattatcttgttgaatgataatttgattcgaagacggattcatgatttgagtatgaggtggatcttctgtactcataattctgtttagggattcagttttgatttgaaaaaaatttgttttcagtatggtgagcggctgttgtgataccatgtgagtaatgaaactctaataattgattaattttcatcctttttctctattcattcaacctgaatatatacaaaagtttgttacaaggataacttctaactacttatgtaacaagcaaccttatcaaaaatacaattagaataacatctaactactacagcttatacatacttatctctatactaacaaattaactaaatatagttaacaagttctgaatcatagtaattttagtctctataatattaacaaatgagggactaaaatacttttcttttcttaaatatgattctattttaaaaagcgggagatagaaaaatttatataatttcttactaaaaaaaagtaaggataattttatttaattctaagaaacaaagcaaaggaaaatgggaaagtttataatttgatctaatttctttgcatgatagacaggtaactgattctcaatacgattctaagatgatccaatataaattatttgtgggaaataggttcacgaaaaagccatttgttggggattcaatgtttattgatttgttttgtgtagtatgttttgcatcaattaattagatcagctagtcatttacatattcttgcaaatttgtgccatttacaaatgaattatctgaatttagtgtaatttgattaatttaattagacgggttttccttcttcagtaatcaacatacacaaagtgaaaggcaaacgagttctagcaatttaattgattaggttcaagttaattaattaatttgataatttatggttcagtccgttactgaaaatttaaaaagaagtagcatgcatctttagattctttctctattttatgcatgccattttctgattaattgtatatgttcgttcttcttgattaagtatttatatttattttttatttgattagtgagctatatatatttttaagagaatcttgatgtatttggacaactaaaaaaattccaagaattgctacaattggagatgaaactgaaaggaaacactatggtgtggatatacgcagtggcaacgtggctctgcttgggatcagccttggggtggtaattcaatgccactgtaagctacacattcccaactgtaagctttaccactgttgatgccacggagcaaatgtacagagagtgttgtagaaaataagaataagtatatatacttatcggcttagtagccaagaagtcttttggtaacctacaactcttaaacatcatatacttaatacataagttacacgcttaatgaatatatatgtaacttacatatttaataggtgactctttaatttcataacttaagcattaaaaactatacataatgattattaaattaaatttagtttttcaacactccttaaatttaatttcagatcaaaccgagtcttgctcttaatctggcaaaatcttcaaactttaatgccttcgtcaaaatatcagcaagttgatcttgcgacttgacatacttcagctctatttccttcttacggatactttctctgatgaaatgatatcttctattaatgtatttactcttaacatgtaaaataggattcttgctcaatgctatagacgatttattatcaataaaaatatcaattggttcttcgtttgaaaccaatagcacctctaacacctttcttaaccatgtggcatgacaaacacaagcagcagcaacaacatacttaacttcacaagttgaaagaattacaatagattacttctttgaactccatgtaaaagcagtctctccaatagaaaacacaaaacccattgtactctttcgatcatcaatatcacctcctcaatcactatcactatagcctactagactgaaactttttgaaggtgagtaaaacaaaccataatctaaagttccttcgacatatctcaaaattcttttaccagccttcatgtgcgaatctgttggtgcctccatgtagcgactaataagtcctactccataaagaatatcaggtctagtacaagtcaagtacctcaagtttccaacaaatctttgaaacaaaattggattcaccttctcagtattatcatatcttgacagctttactccacattcaactagtgtgctgacaagctgacattgctccatgccaaactcctttaagatctttcttacatagctcttgtgagaaataaaaatgccatcattagtttgtttcactttgattcccagataataagacatcaaccctatatctgtcatctcaaattcagcagccatctcctttttgaattattcaatcattgctgaattatttttctgtaaagatcaaatcatcaacatacaagcacaccacaagaacatcaccattctctttcttcttagaataaagagaatattcatgaggatatttcagaaaatttttctttttgaaataatcatcaatcctgttgtaccatgcccttagtacttgcttcaatccatacaaaggtaccttcagttttaaaactttgttttcttcccctctaacaacatatctgaaaggttgttttagatatccttcttcttctaagtatccattcaagaacgccgatttcacgtccatttgaaaaattcttcatctcttttgtgcagctaaagcaatgattaaacaaattatttccaatcgagcaactactacaaagacttcatcatagtcaattccatgttgttggctaaacccttttgcaacaagtcgagccttgtatttctcgacatctccgttttcattctttttaactttttataaatccatttcacaccaacaacttgcttgccttaggaaagattgctaattcccaagtgtcattcttttgaattgattttatttcgtcatctatggctactctccatttttcatttttcatcgcttcttcaaaattcattggttcagtatctgcagaaatacaataatgaacaaaatctaaaacttcatcaatgttagcatatatgtcctacagatttctcatctttgtaggtttttcagatgatcttgaacgacttgaatctccttgaatacttgctggagttcttggaagtgtcaagttctgtacatcaaaattaaaatgttgaacatcttgatctgaataatcaaaaattggaaaaaaattataattttctgattgattctcccaatttcatccatcagcttcctcaaacttgacatctcgactcaggataatctttttgcttgtcggattgtacaacttgtagactttagactttgcatcatatccaacaaaaatgaattttccacttttatcatccagcttaattctggtttcatctgcaacatgaacataagcaacataaccaaaaactctaagatgagaaagagttggctttcttccactccatgcttcctgtggtatagacttctgcaaactctttataggacatatg

SEQ ID NO: 294 - protein, 238 aa, Aequorea macrodactyla
MSKGEELFTGIVPVLIELDGDVHGHKFSVRGEGEGDADYGKLEIKFICTTGKLPVPWPTLVTTLGYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSLQTAISKDRNETRDHMVFLEFFSACGHTHGMDELYK

SEQ ID NO: 295 - DNA, 717 bp, Aequorea macrodactyla
  source 1..717: mol_type = genomic DNA
atgagtaaaggagaagaacttttcactgggattgtcccagttctcattgagttagacggtgatgtccatggacataaattctctgtcagaggagaaggggaaggcgatgcagattatggaaaacttgaaatcaaattcatttgcactactggaaagctaccagttccatggccaacacttgttactacactgggctacggcatccaatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaattcacataatgtatacattatgccggacaaagccaataatggactcaaagtcaatttcaaaattagacacaatatcgaaggtggtggtgtccaacttgctgatcattaccaaacaaatgttccccttggagacggtcctgtccttataccaatcaatcactacctatccttgcaaacagccatttcaaaagatcgaaatgagacgagagatcatatggtgtttctggaatttttctcagcttgtggacatacacatggcatggatgaactatacaaataa

SEQ ID NO: 296 - protein, 489 aa, synthetic construct
MKQSTIALALLPLLFTPVTKARTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 297 - DNA, 7151 bp, synthetic construct
  source 1..7151: mol_type = other DNA
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttttttcgccaggcgcagacttgctgttcttcaggcaatcactcatgtaggtcttacgagcatcccctttcaacgcctgcgccgtcgcctgctgattacaggaggtcatacgctgttgttgtggggttaaagttctctcggcagcgccgacggtggttaaaaaaaccagaccgaaaagcaaggtaaccagtaatgttattttcatagcaccatccctcttcatgttttaaccatgagcgtatgcgcccgtgatctgccattaagtctggttgctaacagcaaaaaaaccacccggcagcgaaaattcactgccgggcgcggttttatttcagccccagagcggctttcatggtgtagaagagatcggtctggtcggtcagtccaacaacattggcggcatgcgggccatacgccgcaatacgcaactgactgccggtatgttcttgtgaatcctcttcggagttcccgtaactcatcaccatcactgcgccatctttggtatttagcgcctgggtgaggcccggagctttggtatccggcgcaacaatctggctggcgtgggcgtgatcagcggtgactatgaccagcgtgttaccctcctttttagcgaattccagcgcccgttgtacggcttcatcgagatcgaccgtctcgccaatttgcccacaaggattcgcagcatgatcctgtttatcgattgacgcaccttcaacttgcaggaaaaagcctttctcatttttactcaacaattcaatggctttgtcggtcatctgcgccagggttggtacactgtcattacgttgcggatttggcgtacaggtgactgcgggcttatcgatattgccatggtacgttgctttcggtcctagccagcgcactggcatattgccgtcagcaaacaggccaagcaggggtttttgctgattcgcttccgtcaccgaattcagtgaggcagcatcgctcaccaactgataaccacgcgcctgtgcctgttcacgcagcgtttttccctgccattcaccagcggttgccgtttcagcaaaggtttttgcgccgccgccaagcgtaacgtcggcacgagcgttaagcagctgttcggtaatcgatccttttccgcctttttccagagcgttacccggacatttttcactggtcgcgctcggaccgtagcatttgcgcgaggtcacatgtgccaccagcgcagcgggcgtggcatcctgcaactctgcggtagaaacgttaccggtcgccagacctgcggcttttgccatttccagaatcgttgggtgatctttttcgtgaatatcgacgcccagcgcgccgttataggttttgacaccggttgaccaggcggttgctgatgcagccgagtcggtgacgtagtccggtttgccggtttttttattcagcgcatagtgagtgtattgcccggtaagcggtaaggcatctatacctttaaaaaagccgcccgcaccttcggcataattacgtgcggcagtaatttccgagtcccccatcccatcgccaatcagcaaaataatattttttgcaggtttatcgctaagagaatcacgcagagcggcagtctgatcacccgttaaacggcgagcaccgccgggtgcagtaatatcgccctgagcagcccggttttccagaacctcgaggctagcatgcatagaaccgccaccaccgtcgacagcggtaccctgcagaggcatttctggtgtccgggcttttgtcacaggggtaaacagtaacggtaagagtgccagtgcaatagtgctttgtttcactttattttctccatgtcgcgtcttatcagggggaattctgtttcctgtgtgaaattgttatccgctcacaattccacacattatacgagccgatgattaattgtcaacagctcatttcagaatatttgccagaaccgttatgatgtcggcgcaaaaaacattatctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 298 - protein, 309 aa, Armoracia rusticana
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 299 - DNA, 955 bp, Armoracia rusticana
  source 1..955: mol_type = genomic DNA
aagcttaaccatgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactcttaataaggatccgaattc

SEQ ID NO: 300 - protein, 6 aa, synthetic construct
HHHHHH

SEQ ID NO: 301 - DNA, 18 bp, synthetic construct
  source 1..18: mol_type = other DNA
catcatcatcaccatcac

SEQ ID NO: 302 - protein, 11 aa, synthetic construct
EQKLISEEDLN

SEQ ID NO: 303 - DNA, 33 bp, synthetic construct
  source 1..33: mol_type = other DNA
gaacaaaaactcatctcagaagaggatctgaat

SEQ ID NO: 304 - protein, 15 aa, synthetic construct
LHHILDAQKMVWNHR

SEQ ID NO: 305 - protein, 238 aa, Aequorea macrodactyla
MSKGEELFTGVVPILVELDGDVHGHKFSVRGEGEGDADYGKLEIKFICTTGKLPVPWPTLVTTLGYGILCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSYQTAISKDRNETRDHMVFLEFFSACGHTHGMDELYK

SEQ ID NO: 306 - DNA, 717 bp, Aequorea macrodactyla
  source 1..717: mol_type = genomic DNA
atgagtaaaggagaagaacttttcactggagttgtcccaattcttgttgaattagatggtgatgtccatggacataaattctctgtcagaggagaaggggaaggcgatgcagattatggaaaacttgaaatcaaattcatttgcactactggaaagctaccagttccatggccaacacttgttactacactgggctatggcatcctatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaactcacataatgtatacattatgccggacaaagccaataatggactcaaagtcaatttcaaaattagacacaatatcgaaggtggtggtgtccaactcgctgatcattaccaaacaaatgttccccttggagacggtcctgtccttataccaatcaatcactacctatcctatcaaacagccatttcaaaagatcgaaatgagacgagagatcatatggtgtttctggaatttttctcagcttgtggacatacacatggcatggatgaactatacaaataa

SEQ ID NO: 307 - protein, 1019 aa, synthetic construct
MADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSADFQLSAGRYHYQLVWCQK

SEQ ID NO: 308 - DNA, 8388 bp, synthetic construct
  source 1..8388: mol_type = other DNA
acgttaagggattttggtcatggacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaattaaggaatcccccaggacccaacgctgcccgagtttgtcagaaagcagaccaaacagcggttggaataatagcgagaacagagaaatagcggcaaaaataatacccgtatcacttttgctgatatggttgatgtcatgtagccaaatcgggaaaaacgggaagtaggctcccatgataaaaaagtaaaagaaaaagaataaaccgaacatccaaaagtttgtgttttttaaatagtacataatggatttccttacgcgaaatacgggcagacatggcctgcccggttattattatttttgacaccagaccaactggtaatggtagcgaccggcgctcagctggaaatccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcagccatttttttctccttacttacttaggatccccgggtaccgagctcgaattggggatcttgaagttcctattccgaagttcctattctctagaaagtataggaacttcagagcgcttttgaagctaattcgagctcggtacccggggatcccccgggctcgactgcattaatgaatcggccaacgcgcggggagaggcggtttgcgtattgggcgctcttccgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtactcgagcagaaagtcaaaagcctccgaccggaggcttttgacttgagggggatcgatcccttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgcagaactcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgccgcgatgaggaagcgggtgccccgcttcttcatcttcgcgcctcgggcctccaggccgcctacctgggcgaaaacatcggtgtttgtggcattcatacggactcctgttgggccagctcgcgcacgggctggcgggtcagcttggcttgaagatcgccacgcattgcggcgatctgcttctcggcatccttgcgcttctgcacgccttcctgctggatgcgaataacgtcctcgacggtcttgatgagcgtcgtctgaacctgcttgagcgtgtccacgtcgatcaccaggcgttggttctccttcgccgtctcgacggacgtgcgatgcagcagggccgcattgcgcttcatcaggtcgttggtggtgtcgtcgatggccgtggccagttcgacggcgttcttctgctcgttgaggctcaaggccagcatgaattgccgcttccacgccggcacggtgatttcgcggatggtgtggaatttatcgaccagcatctggttgttggcctggatcatgcggatggtcggcaggctctgcatggccgaatgttgcaaggcgatcaggtcgccgatgcgcttgtccaggttggcaaccatcgcatcgaggtcggccagctcctgcacgcggccagggtcgttcccgacattgccgcgcagaccctcggcctgctcgcgcagctcggcaaggcggaccttgccggccgcgatgtggacgccaagaaggcggtgttcctcgcgcacggctgcgaacatttcgtcgagcgaggcattgcgctgcgcgatgccttgctgggtggtctgcacttcgctgaccaggtgttcgatctgctcgcgggtcgtgtcgaagcgcgccatgaagcccgtcgaacggacgcggaagcggtcgatcagcgggccaatcaggggcaggcgggaacggttgtcggacaaagggccgacgttcagggaacgggccttggcgacaacctgggtcagtttctcgcctgcttcgtccaggtcgctgttgcgcacctggtccagcaggctatcggcgtagcgggacgtgtgctcggccacgtcgcggccgaactcggcaacggtctgcggactgccgacctcgatccgctgcgcgaccgcatggacttccggcacgtcgctttcctgcaagcccagctcgcgcagggttgccggggtcatgtcgaaggcgacgataggggccttggcgtcgtgcgtcgttttcagtgcgttcatagggttctcccgccgtgttattggttgatgccttccaggctctgcgaaaggctccgcatgagcgcctggtgagctttggccgcctcggcgaccattgccggattcatgttcttggtggtgatgagcgcgagggtgtgctgacgccagacgggcaccaggacggatgccgtttcagagaagcggtccagcatgtccacggcctgcgcccgcgtgagcttcatctgagtgacgctcatttcatgggacgccatgagggttgccaggttggcgagcttgcgcgcgaagcgttcgcgcggcttgtcgaactcgatcacgccggccttggccgcgccggcctcggggttctcgtccaggaactcgcgcccggcttgaatgtaggctctgagccggtctacctcggcctcatgcgtattgagcatgtcatccaaggcgcgcaacgtgtcccgcacgcgctgcgctacgccctcggcttcgtccagcaactggtcgagcgtcttgcgggcgacctgatacctcacctggcgttcaacctcacggccaagcatcttctcgaaccaggtaggcttttccgcgatcttgcgggggtccgcgtcggccagcttcgccacgatctggctgattttgtcggccagcgcggcaactgcgccgtgctccatcagattcgacagctcgttgagggaatccgccccgtcgatgccggccccgtactcgccaatcgtcgccggcgacgcgaagagggcgggcaaaacctcccccttcaatcgcgccatgttcacgctttgttcttccatggtatatctccttcttaaagttaaacaaaattattcggaacccagcatgatattccggaaataccaactaagtcaacggctgatggccaattcggcttcctcgctcactgactcgctgcgctcggtcgttcggctgcggcgagcggtatcagctcactcaaaggcggtaatacggttatccacagaatcaggggataacgcaggaaagaacatgtgagatcgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagctagcttttaaggtttaacggttgtggacaacaagccagggatgtaacgcactgagaagcccttagagcctctcaaagcaattttcagtgacacaggaacacttaacggctgacatgacgctcagtggaacgaaaactc

SEQ ID NO: 309 - protein, 159 aa, synthetic construct
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 310 - DNA, 483 bp, synthetic construct
  source 1..483: mol_type = other DNA
gacccgagcaaagattctaaagcacaagtatctgctgcagaagcaggaattacaggcacatggtataatcagctgggatctacatttattgttacagccggcgcagatggagctcttacaggaacatatgaatctgctgttggaaatgcagaatctagatacgtgcttacaggaagatatgattctgcacctgcaacagatggatccggaacagcacttggatggacagttgcatggaaaaacaattatagaaacgcacatagcgctacaacatggtctggccaatatgtgggaggtgcagaagcaagaattaacacacaatggcttttaacatctggaacaacagaagcaaatgcatggaaaagtactcttgttggacatgatacatttacaaaagttaaacctagcgcagcatctatcgatgcagcgaaaaaagcaggagttaacaatggcaatcctttagatgcagttcaacaataatga

(19) United States
(12) Patent Application Publication
(10) Pub. No.: US 2026/0347500 A1
(43) Pub. Date: Nov. 5, 2026

(54) P32-SPECIFIC ANTIBODIES AND CAR T CELLS FOR THE TREATMENT OF P32 EXPRESSING TUMORS

(71) Applicant: Ramot at Tel-Aviv University Ltd., Tel-Aviv (IL)

(72) Inventors: Dinorah FRIEDMANN-MORVINSKI, Tel-Aviv (IL); Liat ROUSSO-NOORI, Tel-Aviv (IL); Ignacio MASTANDREA, Tel-Aviv (IL); Vered PADLER-KARAVANI, Tel-Aviv (IL); Ron AMON, Tel-Aviv (IL)

(73) Assignee: Ramot at Tel-Aviv University Ltd., Tel-Aviv (IL)

(21) Appl. No.: 19/703,194

(22) Filed: Jun. 10, 2026

Related U.S. Application Data
(63) Continuation of application No. PCT/IL2024/051167, filed on Dec. 10, 2024.
(60) Provisional application No. 63/608,268, filed on Dec. 10, 2023.

(51) Int. Cl. C07K 16/30; C07K 14/705; C12N 5/0783

(52) U.S. Cl. CPC C07K 16/30; C07K 14/7051; C07K 14/70521; C07K 14/70578; C12N 5/0636; C07K 2319/02

(57) ABSTRACT

Provided are isolated p32 antibodies, chimeric antigen receptors comprising same, immune cells (e.g., T-cells) expressing same, nucleic acid constructs encoding same, pharmaceutical compositions comprising same and methods of treating p32-associated cancer using same. Exemplary scFv antibodies which are provided herein comprise SEQ ID NOs: 110, 111 and 100-109. Exemplary CAR sequences which are provided herein are set forth by SEQ ID NO: 82, 88 or 90.

### RELATED APPLICATIONS

This application is a Continuation of PCT Patent Application No. PCT/IL2024/051167 having International filing date of Dec. 10, 2024 which claims the benefit of priority under 35 USC § 119(e) of U.S. Provisional Patent Application No. 63/608,268 filed on Dec. 10, 2023. The contents of the above applications are all incorporated by reference as if fully set forth herein in their entirety.

### SEQUENCE LISTING STATEMENT

The XML file, entitled 107097SequenceListing.xml, created on Mar. 19, 2026, comprising 336,768 bytes, submitted concurrently with the filing of this application is incorporated herein by reference.

### FIELD AND BACKGROUND OF THE INVENTION

The present invention, in some embodiments thereof, relates to p32 specific antibodies and chimeric antigen receptors comprising same and, more particularly, but not exclusively, to methods using same for treating p32-associated cancer in a subject in need thereof.

Malignant gliomas are the most common primary brain tumors and glioblastoma is considered one of the most aggressive malignancies in adult and pediatric patients. Despite decades of research no curative treatment is available and it thus remains associated with a very dismal prognosis.

Immunotherapy has emerged as an important therapeutic modality for a broad range of cancer types. Chimeric antigen receptors (CARs) combine both antibody-like recognition with T cell activating function, endowing the engineered T cells with the capacity to recognize and kill cancer cells. For example, CAR T cells targeting CD19 have shown remarkable success in treating hematological malignancies (“liquid” cancer), but initial attempts to use the same approach in treating “solid” tumors have encountered several challenges.

One of the limitations in solid tumors is the lack of sufficient and specific targets, which can lead to CAR T cells with minimal “off tumor on target” toxicity. Rousso-Noori L., et al., 2021 (Nature Communications 12:3615) have identified p32/gC1qR/HABP/ClqBP (also known as “p32”) to be specifically expressed on the surface of glioma cells, making it an attractive tumor associated antigen for redirected chimeric antigen receptor (CAR) T cell therapy. P32 was shown to be expressed in all types of gliomas, including low and high grade gliomas, pediatric and adult gliomas, as well as in other cancers such as breast, lung, thyroid, ovary, skin, pancreas, mesothelioma, and testis cancer (Valentina Fogal et al., 2008. “Mitochondrial/cell-surface protein p32/gC1qR as a molecular target in tumor cells and tumor stroma”. Cancer Res. 68(17): 7210-8; Ellinor Peerschke et al., 2020. “gC1qR/HABP1/p32 Is a Potential New Therapeutic Target Against Mesothelioma”. Front Oncol. 10:1413). The expression of p32 in human cancers is significantly up-regulated compared to their corresponding normal tissue, and although p32 is primarily expressed in the mitochondria, several studies reported the expression of p32 on the surface of malignant cells (Rousso-Noori L., et al., 2021 (Supra) and PMID: 18757437; Lilach Agemy et al., 2013. “Proapoptotic peptide-mediated cancer therapy targeted to cell surface p32”, Mol Ther December; 21(12): 2195-204; Lauri Paasonen et al., 2016. “New p32/gC1qR Ligands for Targeted Tumor Drug Delivery”; Chembiochem. 17(7): 570-5; Ellinor Peerschke et al., 2020 (Supra).

CAR, which specifically targets p32, was shown to achieve both anti-tumor and anti-angiogenic activity (Rousso-Noori L., et al., 2021 (Supra)). P32 was found to be expressed not only on regular tumor vasculature (neo-angiogenesis, regular endothelial cells from the host that are recruited by the tumor cells to form blood vessels), but also in what was previously defined as “tumor derived endothelial cells” (TDECs), which are formed by glioma stem cells that differentiate into endothelial cells and have the ability to form blood vessels. This TDECs are independent of VEGF (they do not express VEGFR2) and hence are resistant to bevacizumab (Avastin; anti-angiogenic treatment) (Yasushi Soda et al., 2011. Proc Natl Acad Sci USA. 108(11):4274-80).

### SUMMARY OF THE INVENTION

According to an aspect of some embodiments of the present invention there is provided an isolated antibody or a fragment thereof which binds to a human p32 tumor associated antigen (TAA), the antibody comprising a heavy chain variable domain (VH) and a light chain variable domain (VL) each comprising three complementarity-determining regions (CDRs), wherein
(i) the VH CDRs 1, 2 and 3 comprise amino acid sequences SEQ ID NOs: 23, 25 and 27, respectively, and the VL CDRs 1, 2 and 3 comprise amino acid sequences SEQ ID NOs: 30, 32 and 34, respectively;
(ii) the VH CDRs 1, 2 and 3 comprise amino acid sequences SEQ ID NOs: 37, 39 and 41, respectively, and the VL CDRs 1, 2 and 3 comprise amino acid sequences SEQ ID NOs: 44, 46 and 48, respectively;
(iii) the VH CDRs 1, 2 and 3 comprise amino acid sequences SEQ ID NOs: 117, 119 and 121, respectively, and the VL CDRs 1, 2 and 3 comprise amino acid sequences SEQ ID NOs: 124, 126 and 128, respectively;
(iv) the VH CDRs 1, 2 and 3 comprise amino acid sequences SEQ ID NOs: 132, 134 and 136, respectively, and the VL CDRs 1, 2 and 3 comprise amino acid sequences SEQ ID NOs: 139, 141, 143, respectively;
(v) the VH CDRs 1, 2 and 3 comprise amino acid sequences SEQ ID NOs: 147, 149, 151, respectively, and the VL CDRs 1, 2 and 3 comprise amino acid sequences SEQ ID NOs: 154, 156 and 158, respectively;
(vi) the VH CDRs 1, 2 and 3 comprise amino acid sequences SEQ ID NOs: 145, 161 and 163, respectively, and the VL CDRs 1, 2 and 3 comprise amino acid sequences SEQ ID NOs: 166, 168, 170, respectively;
(vii) the VH CDRs 1, 2 and 3 comprise amino acid sequences SEQ ID NOs: 173, 175 and 177 respectively, and the VL CDRs 1, 2 and 3 comprise amino acid sequences SEQ ID NOs: 180, 182 and 184, respectively;
(viii) the VH CDRs 1, 2 and 3 comprise amino acid sequences SEQ ID NOs: 187, 189 and 191, respectively, and the VL CDRs 1, 2 and 3 comprise amino acid sequences SEQ ID NOs: 194, 196 and 198, respectively;
(ix) the VH CDRs 1, 2 and 3 comprise amino acid sequences SEQ ID NOs: 201, 203 and 205, respectively, and the VL CDRs 1, 2 and 3 comprise amino acid sequences SEQ ID NOs: 208, 210 and 212, respectively;
(x) the VH CDRs 1, 2 and 3 comprise amino acid sequences SEQ ID NOs: 215, 217 and 219, respectively, and the VL CDRs 1, 2 and 3 comprise amino acid sequences SEQ ID NOs: 222, 224 and 226, respectively;
(xi) the VH CDRs 1, 2 and 3 comprise amino acid sequences SEQ ID NOs: 229, 231 and 233, respectively, and the VL CDRs 1, 2 and 3 comprise amino acid sequences SEQ ID NOs: 236, 238 and 240, respectively; or
(xii) the VH CDRs 1, 2 and 3 comprise amino acid sequences SEQ ID NOs: 243, 245 and 247, respectively, and the VL CDRs 1, 2 and 3 comprise amino acid sequences SEQ ID NOs: 250, 252 and 254, respectively.

According to an aspect of some embodiments of the present invention there is provided chimeric antigen receptor (CAR) comprising the p32 antibody fragment of some embodiments of the invention and an intracellular signaling domain, the intracellular signaling domain comprising a co-stimulatory signaling region and an activation domain.

According to an aspect of some embodiments of the present invention there is provided a chimeric antigen receptor (CAR) comprising a p32 antibody fragment and an intracellular signaling domain, the intracellular signaling domain comprising a co-stimulatory signaling region and an activation domain, wherein the co-stimulatory signaling region comprises the 41BB amino acid sequence and the activation domain comprises the CD3zeta amino acid sequence.

According to an aspect of some embodiments of the present invention there is provided a nucleic acid construct comprising a polynucleotide comprising a nucleic acid sequence encoding the isolated antibody or the fragment thereof of some embodiments of the invention, wherein the polynucleotide is operably linked to a promoter for directing expression of the nucleic acid sequence in a host cell.

According to an aspect of some embodiments of the present invention there is provided a nucleic acid construct comprising a polynucleotide comprising a nucleic acid sequence encoding the CAR of some embodiments of the invention, wherein the polynucleotide is operably linked to a promoter for directing expression of the nucleic acid sequence in a host cell.

According to an aspect of some embodiments of the present invention there is provided an isolated T lymphocyte cell (T cell) transduced to express the antibody or the fragment thereof of some embodiments of the invention or the CAR of some embodiments of the invention.

According to an aspect of some embodiments of the present invention there is provided a pharmaceutical composition comprising the isolated antibody or the fragment thereof of some embodiments of the invention, the nucleic acid construct of some embodiments of the invention, or the isolated genetically modified immune cell (e.g., T cell) of some embodiments of the invention, and a pharmaceutically acceptable carrier.

According to an aspect of some embodiments of the present invention there is provided a method of treating a subject diagnosed with a p32-associated cancer, the method comprising administering to the subject a therapeutically effective amount of the isolated antibody or the fragment thereof of some embodiments of the invention, the nucleic acid construct of some embodiments of the invention, or the isolated genetically modified immune cell (e.g., T cell) of some embodiments of the invention, thereby treating the subject diagnosed with a p32-associated cancer.

According to an aspect of some embodiments of the present invention there is provided a method of inhibiting angiogenesis in a p32-associated cancer, comprising contacting cells of a p32-associated cancer tumor microenvironment (TME) with the isolated antibody or the fragment thereof of some embodiments of the invention, the nucleic acid construct of some embodiments of the invention, or the isolated genetically modified immune cell (e.g., T cell) of some embodiments of the invention, thereby inhibiting angiogenesis in the p32-associated cancerous tumor.

According to an aspect of some embodiments of the present invention there is provided a method of inhibiting expression of p32 on a tumor cell, a macrophage residing in a tumor microenvironment or a tumor-derived endothelial cell, contacting the tumor cell, the macrophage or the tumor-derived endothelial cell with the isolated antibody or the fragment thereof of some embodiments of the invention, the nucleic acid construct of some embodiments of the invention, or the isolated genetically modified immune cell (e.g., T cell) of some embodiments of the invention, thereby inhibiting expression of the p32 on the tumor cell, the macrophage residing in the tumor microenvironment or the tumor-derived endothelial cell.

According to an aspect of some embodiments of the present invention there is provided the isolated antibody or the fragment thereof of some embodiments of the invention, the nucleic acid construct of some embodiments of the invention, or the isolated genetically modified immune cell (e.g., T cell) of some embodiments of the invention, for use in the treatment of a p32-associated cancer.

According to some embodiments of the invention, the antibody of
(i) further comprises heavy chain variable domain framework regions (FRs) 1, 2, 3 and 4 comprising amino acids SEQ ID NOs: 22, 24, 26 and 28, respectively;
(ii) further comprises heavy chain variable domain FRs 1, 2, 3 and 4 comprising amino acids SEQ ID NOs: 36, 38, 40 and 42, respectively;
(iii) further comprises heavy chain variable domain FRs 1, 2, 3 and 4 comprising amino acids SEQ ID NOs: 116, 118, 120 and 122, respectively;
(iv) further comprises heavy chain variable domain FRs 1, 2, 3 and 4 comprising amino acids SEQ ID NOs: 131, 133, 135 and 137, respectively;
(v) further comprises heavy chain variable domain FRs 1, 2, 3 and 4 comprising amino acids SEQ ID NOs: 146, 148, 150 and 152, respectively;
(vi) further comprises heavy chain variable domain FRs 1, 2, 3 and 4 comprising amino acids SEQ ID NOs: 130, 160, 162 and 164, respectively;
(vii) further comprises heavy chain variable domain FRs 1, 2, 3 and 4 comprising amino acids SEQ ID NOs: 172, 174, 176 and 178, respectively;
(viii) further comprises heavy chain variable domain FRs 1, 2, 3 and 4 comprising amino acids SEQ ID NOs: 186, 188, 190 and 192, respectively;
(ix) further comprises heavy chain variable domain FRs 1, 2, 3 and 4 comprising amino acids SEQ ID NOs: 200, 202, 204 and 206, respectively;
(x) further comprises heavy chain variable domain FRs 1, 2, 3 and 4 comprising amino acids SEQ ID NOs: 214, 216, 218 and 220, respectively;
(xi) further comprises heavy chain variable domain FRs 1, 2, 3 and 4 comprising amino acids SEQ ID NOs: 228, 230, 232 and 234, respectively; or
(xii) further comprises heavy chain variable domain FRs 1, 2, 3 and 4 comprising amino acids SEQ ID NOs: 242, 244, 246 and 248, respectively.

According to some embodiments of the invention, the antibody of
(i) further comprises light chain variable domain framework regions (FRs) 1, 2, 3 and 4 comprising amino acids SEQ ID NOs: 29, 31, 33 and 35, respectively;
(ii) further comprises light chain variable domain FRs 1, 2, 3 and 4 comprising amino acids SEQ ID NOs: 43, 45, 47 and 49, respectively;
(iii) further comprises light chain variable domain FRs 1, 2, 3 and 4 comprising amino acids SEQ ID NOs: 123, 125, 127 and 129, respectively;
(iv) further comprises light chain variable domain FRs 1, 2, 3 and 4 comprising amino acids SEQ ID NOs: 138, 140, 142 and 144, respectively;
(v) further comprises light chain variable domain FRs 1, 2, 3 and 4 comprising amino acids SEQ ID NOs: 153, 155, 157 and 159, respectively;
(vi) further comprises light chain variable domain FRs 1, 2, 3 and 4 comprising amino acids SEQ ID NOs: 165, 167, 169 and 171, respectively;
(vii) further comprises light chain variable domain FRs 1, 2, 3 and 4 comprising amino acids SEQ ID NOs: 179, 181, 183, 185, respectively;
(viii) further comprises light chain variable domain FRs 1, 2, 3 and 4 comprising amino acids SEQ ID NOs: 193, 195, 197 and 199, respectively;
(ix) further comprises light chain variable domain FRs 1, 2, 3 and 4 comprising amino acids SEQ ID NOs: 207, 209, 211 and 213, respectively;
(x) further comprises light chain variable domain FRs 1, 2, 3 and 4 comprising amino acids acids SEQ ID NOs: 221, 223, 225 and 227, respectively;
(xi) further comprises light chain variable domain FRs 1, 2, 3 and 4 comprising amino acids SEQ ID NOs: 235, 237, 239 and 241, respectively; or
(xii) further comprises light chain variable domain FRs 1, 2, 3 and 4 comprising amino acids SEQ ID NOs: 249, 251, 253 and 255, respectively.

According to some embodiments of the invention, the antibody comprises a heavy chain variable domain comprising an amino acid sequence at least 90% identical to the amino acid sequence selected from the group consisting of SEQ ID NOs: 92, 96, 256, 258, 260, 262, 264, 266, 268, 270, 272 and 274.

According to some embodiments of the invention, the antibody comprises a heavy chain variable domain comprising the amino acid sequence selected from the group consisting of SEQ ID NOs: 92, 96, 256, 258, 260, 262, 264, 266, 268, 270, 272 and 274.

According to some embodiments of the invention, the antibody comprises a light chain variable domain comprising an amino acid sequence at least 90% identical to the amino acid sequence selected from the group consisting of SEQ ID NOs: 93, 97, 257, 259, 261, 263, 265, 267, 269, 271, 273 and 275.

According to some embodiments of the invention, the antibody comprises a light chain variable domain comprising the amino acid sequence selected from the group consisting of SEQ ID NOs: 93, 97, 257, 259, 261, 263, 265, 267, 269, 271, 273 and 275.

According to some embodiments of the invention, the isolated antibody or the fragment thereof of some embodiments of the invention, being a single chain Fv (scFv).

According to some embodiments of the invention, the scFv is selected from the group consisting of SEQ ID NOs: 110, 111 and 100-109.

According to some embodiments of the invention, the isolated antibody or the fragment thereof of some embodiments of the invention, being conjugated to a functional moiety.

According to some embodiments of the invention, the functional moiety comprises a therapeutic moiety.

According to some embodiments of the invention, the functional moiety is a therapeutic moiety.

According to some embodiments of the invention, the functional moiety comprises a detectable moiety.

According to some embodiments of the invention, the functional moiety is a detectable moiety.

According to some embodiments of the invention, the co-stimulatory signaling region comprises CD28, 41BB, OX40, ICOS, CD27, MYD88-CD40, Toll like-Receptors (TLRs) or KIR2DS2.

According to some embodiments of the invention, the activation domain comprises FcRgamma or CD3zeta.

According to some embodiments of the invention, the CAR comprising the amino acid sequence as set forth by SEQ ID NO: 88 or 90.

According to some embodiments of the invention, the p32 antibody comprises a heavy chain CDRs 1, 2, and 3 as set forth by SEQ ID NOs: 9, 11, and 13, respectively.

According to some embodiments of the invention, the p32 antibody comprises light chain CDRs 1, 2, and 3 as set forth by SEQ ID NOs: 16, 18 and 20, respectively.

According to some embodiments of the invention, the CAR further comprises heavy chain variable domain comprising framework regions (FRs) 1, 2, 3 and 4 as set forth by SEQ ID NOs: 8, 10, 12, and 14, respectively.

According to some embodiments of the invention, the CAR further comprises light chain variable domain comprising framework regions (FRs) 1, 2, 3 and 4 as set forth by SEQ ID NOs: 15, 17, 19 and 21, respectively.

According to some embodiments of the invention, the CAR comprising the amino acid sequence as set forth by SEQ ID NO: 82.

According to some embodiments of the invention, the CAR further comprises a hinge sequence, a transmembrane domain and/or a cell surface or secreted immunomodulatory molecule.

According to some embodiments of the invention, the hinge sequence comprises CD8 hinge, CD28 hinge, IgG1 hinge or IgG4 hinge sequence.

According to some embodiments of the invention, the transmembrane domain (TMD) comprises a CD3-zeta TMD, CD8alpha TMD, CD4 TMD, CD28 TMD or ICOS TMD.

According to some embodiments of the invention, the cell surface or secreted immunomodulatory molecule enhances T cell function or modifies tumor microenvironment.

According to some embodiments of the invention, the isolated antibody or the fragment thereof, or the CAR of some embodiments of the invention, being soluble.

According to some embodiments of the invention, the isolated antibody or the fragment thereof of some embodiments of the invention, or the CAR of some embodiments of the invention, being insoluble.

According to some embodiments of the invention, the host cell is a T lymphocyte cell.

According to some embodiments of the invention, the contacting is effected in-vivo by administration of the T cells into a subject.

According to some embodiments of the invention, the contacting is effected in-vitro.

According to some embodiments of the invention, the cells of the p32-associated cancer TME are endothelial cells.

According to some embodiments of the invention, the endothelial cells are tumor-derived endothelial cells.

According to some embodiments of the invention, the p32-associated cancer is glioblastoma, breast cancer, lung cancer, thyroid cancer, ovary cancer, skin cancer, pancreatic cancer or testicular cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings.

With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a histogram showing the binding of different yeast clones presenting anti-p32 scFv from the 3^{rd} round of panning compared to native p32 scFv (“native”) and secondary antibody control. 25 ng of p32-Bio was used for panning and flow cytometry analysis.

FIG. 2 is a graph showing the binding capacity of selected p32 scFv clones—expressing yeast cells. Clones (#1, 5, 8, 11, and 20, as well as “native” p32) were examined against 6 serial dilutions of p32-biotin by flow cytometry. It is noted that clones #1, 5, 8, 11, and 20 exhibit significantly improved binding to p32 as compared to the “native” p32 scFv clone.

FIGS. 3A-E depict proliferation and cytotoxic effects of human p32-41BB-CD3zeta CAR T cells on human glioma cells. FIGS. 3A-C depict the structure of CAR T constructs which include the p32 scFV sequence (either the native p32 scFv (FIG. 3A), or the mutated p32 scFv from clones #8 (FIG. 3B) or #20 (FIG. 3C)), along with the 41BB and CD3z sequences. FIG. 3D—CellTracer™ dilution assay of labeled p32 CAR T human lymphocytes co-cultured with U251 glioma cells for 72 hours (Effector to Target (E:T)=1:1). FIG. 3E—Cytotoxic action of human p32 CAR T cells against U251 cells was measured by the amount of residual GFP⁺ tumor cells after co-culture (E:T=2:1) with native p32 CAR, selected p32 clones CAR T (native p32 CAR clone, #8 p32 CAR clone and #20 p32 CAR clone) and un-transduced (UT) T cells (control) for 72 hours. It is noted that while the native p32 CAR T cells significantly reduced the percentage of residual tumor cells, the #8 and #20 p32 CAR T cells exhibit a significantly higher effect on reducing the percentage of residual tumor cells compared to the native p32 CAR T cells.

FIGS. 4A-K depict the effect of the isolated p32-CD28-FcRgamma CAR T (also referred to as “p32-CD28g CAR T”) and p32-41BB-CD3zeta CAR T (also referred to as “p32-41BBz CAR T”) in killing cancer cells. FIGS. 4A-B—Schematic representations of p32 CAR constructs: FLAG-p32-CD28g-mCherry (FIG. 4A) and FLAG-P32-41BBz-mCherry (FIG. 4B) CAR constructs. FIG. 4C—Primary human T cells were either un-transduced (UT) or transduced with the FLAG-p32-CD28g-mCherry CAR or FLAG-p32-41BBz-mCherry CAR. Transduction rates were evaluated by flow cytometry via FLAG expression, using an anti-FLAG primary antibody and a secondary antibody conjugated to Alexa-647. FIG. 4D—Frequency of CD4+/CD8+ T cells on day 3 after transduction analyzed by flow cytometry. FIG. 4E—Analysis of CD25 activation marker expression on day 6 after cell transduction. “US Ctrl”=unstained control T cells; “UT cells”=un-transduced T cells; “p32-CD28g CAR T cells”—T cells transduced with the native p32-CD28g CAR (schematically shown in FIG. 4A); “p32-41BBz CAR T cells”=T cells transduced with the native p32-41BB CD3z CAR (schematically shown in FIG. 4B). FIGS. 4F-G—Human target GBM83 (patient-derived glioma cells) luciferase-expressing cells were co-cultured with control UT cells, p32-CD28g-mCherry or p32-41BBz-mCherry CAR T-cells at the indicated E:T ratios. Eighteen hours later, the cytotoxic action of the human CAR T cells was measured by quantifying luciferase activity in tumor target cells (FIG. 4F; data represents mean±SD). Statistical analysis is shown for 5:1 E:T ratio (FIG. 4G). FIGS. 4H-K: GBM83 GFP+ cells were co-cultured with control UT cells (FIG. 4H), p32-CD28g-mCherry CAR T-cells (FIG. 4I) or p32-41BBz-mCherry CAR T-cells (FIG. 4J) for 72 hours in a 3:1 ratio and analyzed by flow cytometry. The cytotoxic action exerted by the p32-CD28-FcRgamma and p32-41BB-CD3zeta against GBM83 glioma cells was measured by the amount of residual GFP+ tumor cells after co-culture (13% and 5%, respectively) as well as the expansion of the T cell population (87% and 95%). Representative plots are shown. FIG. 4K—Quantification of % GBM83 cells in the co-cultures (described in FIGS. 4H-J). The results show that p32-41BBz-mCherry CAR T-cells have an improved function in terms of activation, proliferation and killing capability when compared to p32-CD28g-mCherry CAR T-cells.

FIGS. 5A-I depict that tumor infiltrating macrophages are being targeted by p32 CAR-T therapy. FIG. 5A—p32 cell surface expression analyzed by flow cytometry (FACS) in RAW 264.7 macrophage-like cell line after exposure to 005 conditioned media (CM; a conditioned medium of mouse glioblastoma cells termed “005”) or in the presence of “N2” (N2 supplemented stem cell media alone, composition of the media is described in Rousso-Noori L., et al., 2021 (Supra)). The control in FIG. 5A is RAW 264.7 cell exposed only to the media used to grow 005 cells (fresh from the bottle), showing that the media itself does not induce the expression of p32 at the surface of RAW 264.7 cells. Only when the latter are exposed to CM they express p32. FIGS. 5B-C: Representative 005 tumors and wild type brains were dissociated and analyzed by FACS for p32 surface expression on CD45+/CD11b+/CD68+ cells (“SSC”=side scatter; measures scatter at a ninety-degree angle relative to the laser in flow cytometry). The results show that macrophages that were removed (isolated) from brain tumors with the 005 glioblastoma cells are positive for p32 (more than 89%) as compared to absence of such p32+ macrophages (less than 1%) in brains of wild-type mice (without the glioblastoma cells). FIG. 5D shows same results as depicted in FIG. 5C in an histogram graph and gating on the CD68+ population in the 005 dissociated tumor (Isotype=IgG isotype-PE control antibody); FIGS. 5E-F: Tumor sections (40 μm) of mice, which were treated with either mSP6 (an irrelevant control CAR T cells) or with p32 CAR T cell (native p32 scFV-CD28-FcRγ CAR T cells), were stained with CD68 antibody and analyzed by confocal microscope (FIG. 5E). Quantification was assessed by Fiji/ImageJ (FIG. 5F). Each dot shown in the graph represents average of three measurements done per slide. A total of 5-6 slides originated in three different mice and were stained per group. Unpaired t test was used. Two-tailed P value is shown. The results demonstrate that CAR T cells, which were administered to the mice by intra-venous administration, and which comprise the p32 scFV reached the site of tumor (i.e., the brain) and bind therein to the p32 tumor-associated antigen (TAA). Furthermore, the CAR T cells were able to eliminate not only tumor cells and TDEC as previously described (Rousso-Noori L., et al., 2021 (Supra)) but also tumor associated macrophages (TAMs). FIGS. 5G-I: p32 expression was analyzed in bone-marrow macrophages (CD45+/CD11b+/CD68+) of wild type and 005-tumor-bearing mice (FIGS. 5G-H). FIG. 5I—a histogram presenting quantification of the results shown in FIGS. 5G and 5H, demonstrating that there is no difference between the circulating macrophages (from bone marrow) of mice bearing the glioblastoma tumor (005) and control wild-type mice (healthy mice, without the glioblastoma). Altogether, these results clearly show the specificity of the p32+ macrophages to the tumor environment and their absence in the peripheral bone marrow.

FIGS. 6A-C depict the sequences of the Native scFvp32-CD28-gamma CAR. FIG. 6A—The amino acid of the native scFvp32-CD28-gamma CAR, set forth by SEQ ID NO: 50. The Native scFvp32-CD28-gamma CAR comprises an OncoM signal peptide (SEQ ID NO: 51; black letters highlighted in green), a FLAG tag (SEQ ID NO: 52; black letters highlighted in grey), the native scFvp32 VH sequence (SEQ ID NO: 53; white letters highlighted in Teal), a linker (SEQ ID NO: 54; white letters highlighted in Burgundy), the native scFv32 VL (SEQ ID NO: 55; white letters highlighted in Forest color), a His 6× tag (SEQ ID NO: 56; red letters), a Myc tag (SEQ ID NO: 57; Olympic color), and a CD28 hinge (SEQ ID NO: 58), CD28 transmembrane (SEQ ID NO: 59) and CD28 cytoplasmic (SEQ ID NO: 60) domains (black letters highlighted in yellow, italics, bolded and underlined, respectively), an FcRIgamma chain (SEQ ID NO: 61, black letters highlighted in Cyan), a T2A sequence (SEQ ID NO: 62; black letters highlighted in Olive color), and an MCherry sequence (SEQ ID NO: 63; white letters highlighted in red).

FIG. 6B—The nucleic acid sequence encoding the Native scFvp32-CD28-gamma CAR, set forth by SEQ ID NO: 64. SEQ ID NO: 64 comprises the coding sequence of an OncoM signal peptide (SEQ ID NO: 65; black letters highlighted in green), the coding sequence of a FLAG tag (SEQ ID NO: 66; black letters highlighted in grey), the coding sequence of the native scFvp32 VH sequence (SEQ ID NO: 67; white letters highlighted in Teal), the coding sequence of a linker (SEQ ID NO: 68; white letters highlighted in Bargundy), the coding sequence of the native scFv32 VL (SEQ ID NO: 69; white letters highlighted in Forest color), the coding sequence of a His 6× tag (SEQ ID NO: 70; red letters), the coding sequence of a Myc tag (SEQ ID NO: 71; Olympic color), the coding sequence of a CD28 hinge (SEQ ID NO: 72), a CD28 transmembrane (SEQ ID NO: 73) and a CD28 cytoplasmic (SEQ ID NO: 74) domains (black letters highlighted in yellow, italics, bolded and underlined, respectively), the coding sequence of an FcRIgamma chain (SEQ ID NO: 75, black letters highlighted in Cyan), the coding sequence of a T2A sequence (SEQ ID NO: 76; black letters highlighted in Olive color), and the coding sequence of an MCherry sequence (SEQ ID NO: 77; white letters highlighted in red).

FIG. 6C depicts the color coded domains of the Native scFvp32-CD28-gamma CAR sequences described and shown in FIGS. 6A and 6B.

FIGS. 7A-C depict the sequences of Native scFvp32-41BB-zeta CAR. FIG. 7A—The amino acid of the Native scFvp32-41BB-zeta CAR, set forth by SEQ ID NO: 82. The Native scFvp32-41BB-zeta CAR comprises an OncoM signal peptide (SEQ ID NO: 51; black letters highlighted in green), a FLAG tag (SEQ ID NO: 52; black letters highlighted in grey), the native scFvp32 V_{H} sequence (SEQ ID NO: 53; white letters highlighted in Teal), a linker (SEQ ID NO: 54; white letters highlighted in Bargundy), the native scFv32 V_{L} (SEQ ID NO: 55; white letters highlighted in Forest color), a His 6× tag (SEQ ID NO: 56; red letters), a Myc tag (SEQ ID NO: 57; Olympic color), the CD8 hinge (SEQ ID NO: 78; black letters highlighted in yellow) and CD8 transmembrane (SEQ ID NO: 79; Orange letters) domains, the 41BB sequence (SEQ ID NO: 80), a CD3zeta sequence (SEQ ID NO: 81), a T2A sequence (SEQ ID NO: 62; black letters highlighted in Olive color), and an MCherry sequence (SEQ ID NO: 63; white letters highlighted in red).

FIG. 7B—The nucleic acid coding sequence of the Native scFvp32-41BB-zeta CAR (SEQ ID NO: 87) comprises the coding sequence of the OncoM signal peptide (SEQ ID NO: 65; black letters highlighted in green), the coding sequence of a FLAG tag (SEQ ID NO: 66; black letters highlighted in grey), the coding sequence of the native scFvp32 V_{H} sequence (SEQ ID NO: 67; white letters highlighted in Teal), the coding sequence of a linker (SEQ ID NO: 68; white letters highlighted in Burgundy), the coding sequence of the native scFv32 V_{L} (SEQ ID NO: 69; white letters highlighted in Forest color), the coding sequence of a His 6× tag (SEQ ID NO: 70; red letters), the coding sequence of a Myc tag (SEQ ID NO: 71; Olympic color), the coding sequence of the CD8 hinge (SEQ ID NO: 83; black letters highlighted in yellow), the coding sequence of CD8 transmembrane (SEQ ID NO: 84; Orange letters) domains, the coding sequence of the 41BB sequence (SEQ ID NO: 85), the coding sequence of CD3zeta sequence (SEQ ID NO: 86), the coding sequence of a T2A sequence (SEQ ID NO: 76; black letters highlighted in Olive color), and the coding sequence of an MCherry sequence (SEQ ID NO: 77; white letters highlighted in red).

FIG. 7C depicts the color coded domains of the Native/WT scFvp32-41BB-zeta CAR sequences shown in FIGS. 7A and 7B.

FIGS. 8A-C depict the sequences of scFvp32 col #8-41BB-zeta CAR. FIG. 8A—The amino acid of the scFvp32 col #8-41BB-zeta CAR (SEQ ID NO: 88). The scFvp32 col #8-41BB-zeta CAR comprises an OncoM signal peptide (SEQ ID NO: 51; black letters highlighted in green), a FLAG tag (SEQ ID NO: 52; black letters highlighted in grey), the scFvp32 col #8 VH sequence (SEQ ID NO: 92; white letters highlighted in Teal), a linker (SEQ ID NO: 54; white letters highlighted in Bargundy), the scFvp32 col #8 VL (SEQ ID NO: 93; black letters highlighted in Olive color), a His 6× tag (SEQ ID NO: 56; red letters), a Myc tag (SEQ ID NO: 57; Olympic color), the CD8 hinge (SEQ ID NO: 78; black letters highlighted in yellow) and CD8 transmembrane (SEQ ID NO: 79; Orange letters) domains, the 41BB sequence (SEQ ID NO: 80; black letters in Cyan), a CD3zeta sequence (SEQ ID NO: 81, black letters in purple), a T2A sequence (SEQ ID NO: 62; black letters highlighted in Olive color), and an MCherry sequence (SEQ ID NO: 63; white letters highlighted in red).

FIG. 8B—The nucleic acid sequence encoding the scFvp32 col #8-41BB-zeta CAR (SEQ ID NO: 89) comprises the coding sequence of an OncoM signal peptide (SEQ ID NO: 65; black letters highlighted in green), the coding sequence of a FLAG tag (SEQ ID NO: 66; black letters highlighted in grey), the coding sequence of the scFvp32 col #8 VH sequence (SEQ ID NO: 94; white letters highlighted in Teal), the coding sequence of a linker (SEQ ID NO: 68; white letters highlighted in Bargundy), the coding sequence of the scFvp32 col #8 VL (SEQ ID NO: 95; black letters highlighted in Olive color), the coding sequence of a His 6× tag (SEQ ID NO: 70; red letters), the coding sequence of a Myc tag (SEQ ID NO: 71; Olympic color), the coding sequence of the CD8 hinge (SEQ ID NO: 83; black letters highlighted in yellow), the coding sequence of the CD8 transmembrane (SEQ ID NO: 84; Orange letters) domains, the coding sequence of the 41BB sequence (SEQ ID NO: 85; black letters in Cyan), the coding sequence of the CD3zeta sequence (SEQ ID NO: 86, black letters in purple), the coding sequence of a T2A sequence (SEQ ID NO: 76; black letters highlighted in Olive color), and the coding sequence of an MCherry sequence (SEQ ID NO: 77; white letters highlighted in red).

FIG. 8C depicts the color coded domains of the scFvp32 col #8-41BB-zeta CAR sequences shown in FIGS. 8A and 8B.

FIGS. 9A-D depict the sequences of scFvp32 col #20-41BB-zeta CAR.

FIG. 9A—The amino acid of the scFvp32 col #20-41BB-zeta CAR, set forth by SEQ ID NO: 90. The scFvp32 col #20-41BB-zeta CAR comprises an OncoM signal peptide (SEQ ID NO: 51; black letters highlighted in green), a FLAG tag (SEQ ID NO: 52; black letters highlighted in grey), the scFvp32 col #20 VH sequence (SEQ ID NO: 96; black letters highlighted in Teal), a linker (SEQ ID NO: 54; white letters highlighted in Burgundy), the scFvp32 col #20 VL (SEQ ID NO: 97; black letters highlighted in Olive color), a His 6× tag (SEQ ID NO: 56; red letters), a Myc tag (SEQ ID NO: 57; Olympic color), the CD8 hinge (SEQ ID NO: 78; black letters highlighted in yellow), a CD8 transmembrane (SEQ ID NO: 79; Orange letters), the 41BB sequence (SEQ ID NO: 80; black letters in Cyan), a CD3zeta sequence (SEQ ID NO: 81, black letters in purple highlight), a T2A sequence (SEQ ID NO: 62; black letters highlighted in Olive highlight), and an MCherry sequence (SEQ ID NO: 63; white letters highlighted in red).

FIG. 9B depicts the color coded domains of the scFvp32 col #20-41BB-zeta CAR sequences shown in FIG. 9A.

FIG. 9C—The nucleic acid sequence encoding the scFvp32 col #20-41BB-zeta CAR (SEQ ID NO: 91). The coding sequence of scFvp32 col #20-41BB-zeta CAR comprises the coding sequence of an OncoM signal peptide (SEQ ID NO: 65; black letters highlighted in green), the coding sequence of a FLAG tag (SEQ ID NO: 66; black letters highlighted in grey), the coding sequence of the scFvp32 col #20 VH sequence (SEQ ID NO: 98; white letters highlighted in Teal), the coding sequence of a linker (SEQ ID NO: 68; white letters highlighted in Burgundy), the coding sequence of the scFvp32 col #20 VL (SEQ ID NO: 99; white letters highlighted in Olive color), the coding sequence of a His 6× tag (SEQ ID NO: 70; red letters), the coding sequence of a Myc tag (SEQ ID NO: 71; Olympic color), the coding sequence of the CD8 hinge (SEQ ID NO: 83; black letters highlighted in yellow) and CD8 transmembrane (SEQ ID NO: 84; Orange letters) domains, the coding sequence of the 41BB sequence (SEQ ID NO: 85; black letters in Cyan), the coding sequence of the CD3zeta sequence (SEQ ID NO: 86, black letters in purple highlight), the coding sequence of a T2A sequence (SEQ ID NO: 76; black letters highlighted in Olive highlight), and the coding sequence of an MCherry sequence (SEQ ID NO: 77; white letters highlighted in red).

FIG. 9D depicts the color coded domains of the scFvp32 col #20-41BB-zeta CAR sequences shown in FIG. 9C.

FIGS. 10A-L depict sequence alignments between the amino acid sequence of the native p32 scFv sequence (SEQ ID NO: 110) and the scFv of clone #1 (FIG. 10A), 5 (FIG. 10B), 9 (FIG. 10C), 10 (FIG. 10D), 11 (FIG. 10E), 12 (FIG. 10F), 14 (FIG. 10G), 15 (FIG. 10H), 17 (FIG. 10I), 24 (FIG. 10J), 8 (FIG. 10K) and 20 (FIG. 10L).

FIGS. 11A-F depict the sequences of scFv of p32 native, p32 clone #8 and p32 clone #20 with colored domains: VH sequences—white letters highlighted in Teal; linker sequences—white letters highlighted in Burgundy; and VL sequences—white letters highlighted in in Forest color. FIG. 11A—Amino acid sequence of the p32 native scFv; FIG. 11B—Nucleic acid sequence of the p32 native scFv; FIG. 11C—Amino acid sequence of the p32 clone #8 scFv; FIG. 11D—Nucleic acid sequence of the p32 clone #8 scFv; FIG. 11E—Amino acid sequence of the p32 clone #20 scFv; FIG. 11F—Nucleic acid sequence of the p32 clone #20 scFv.

### DESCRIPTION OF SPECIFIC EMBODIMENTS OF THE INVENTION

The present invention, in some embodiments thereof, relates to p32 specific antibodies and chimeric antigen receptors comprising same and, more particularly, but not exclusively, to methods using same for treating p32-associated cancer in a subject in need thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

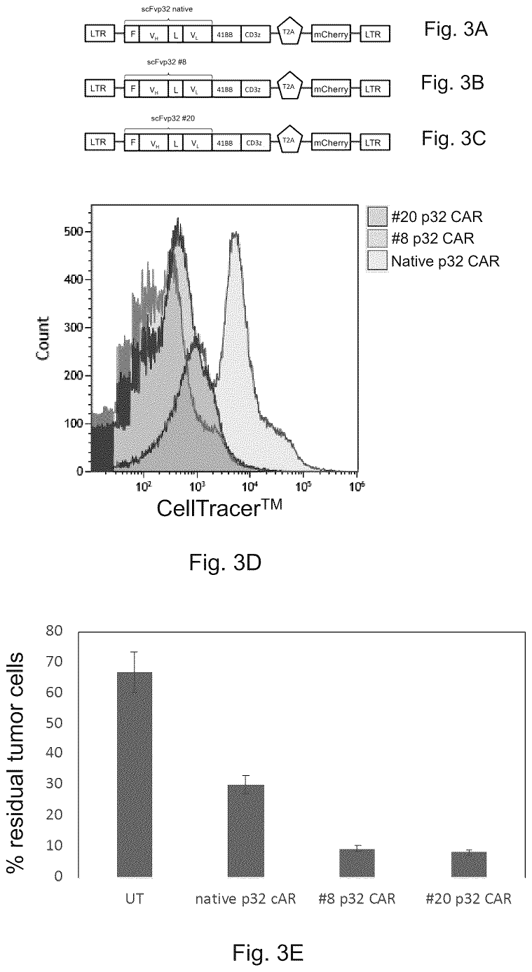

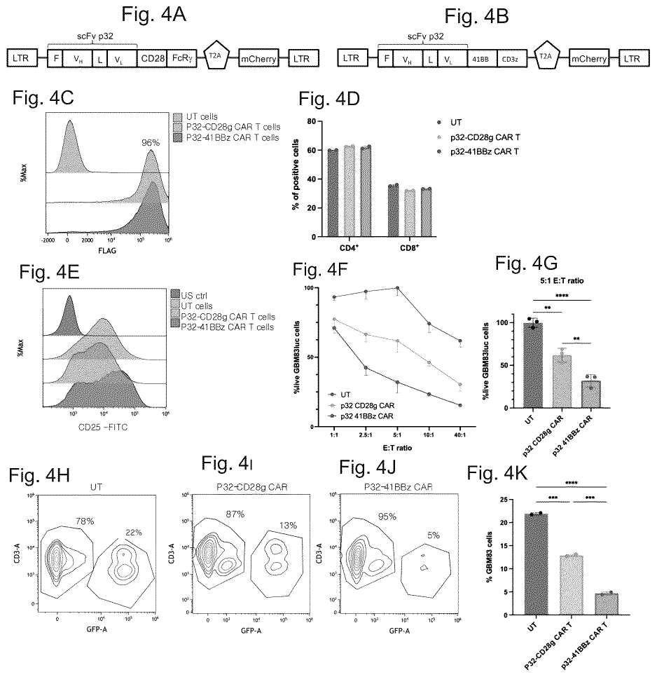

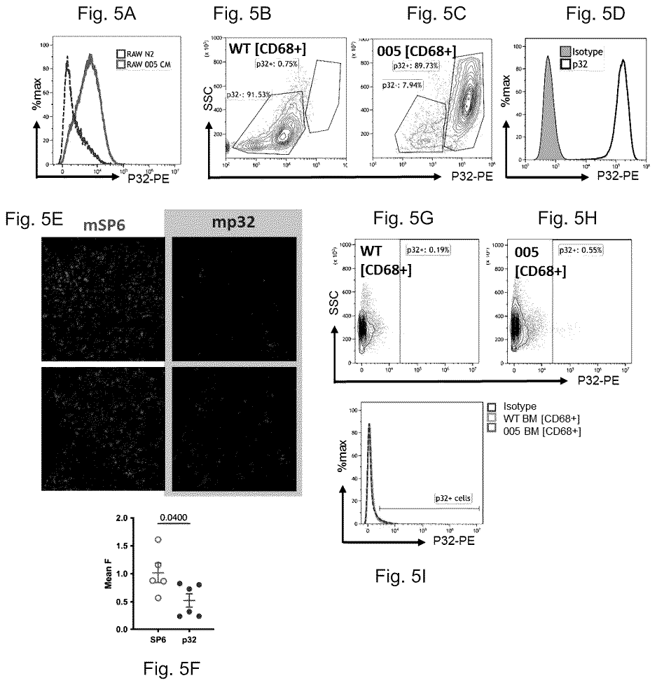

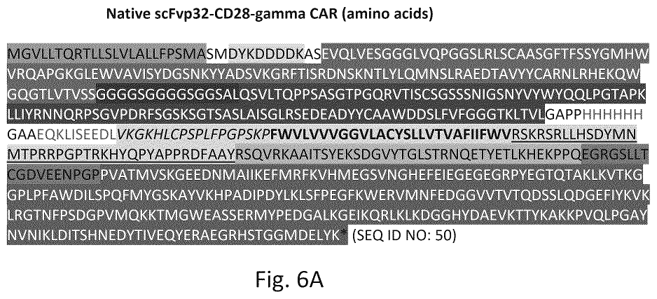

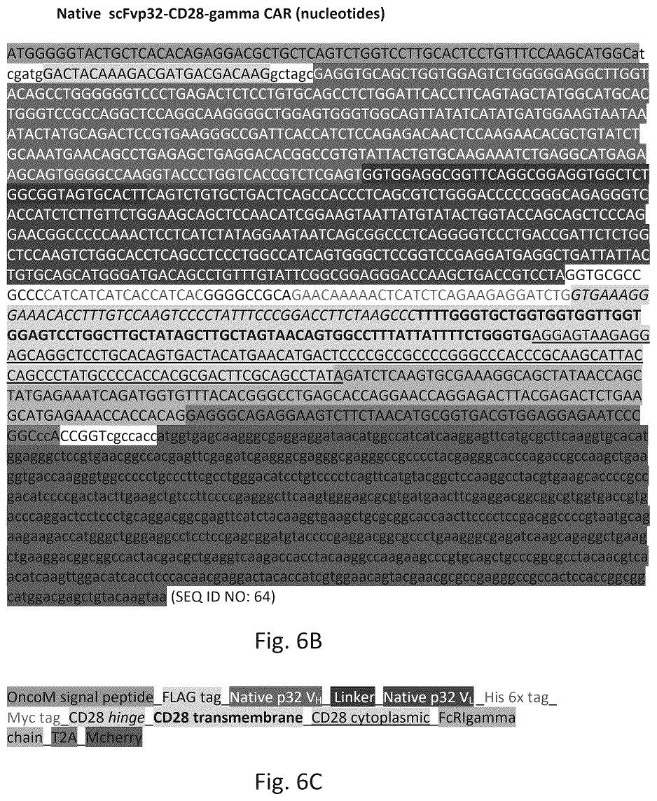

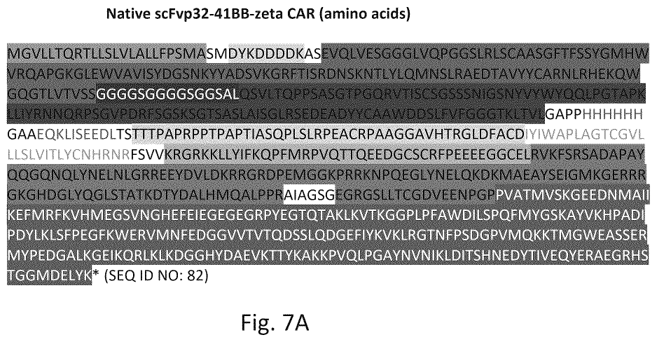

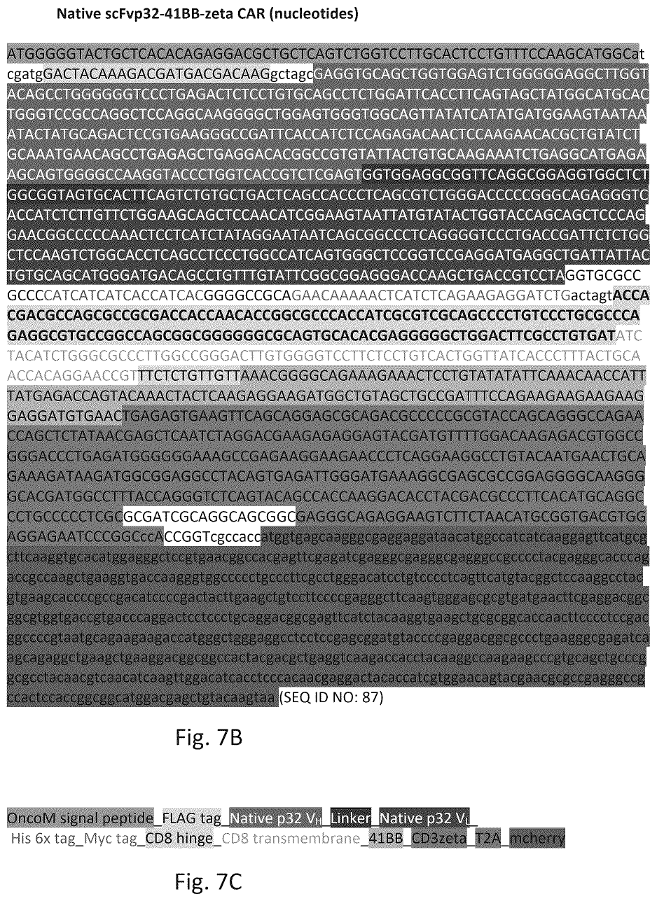

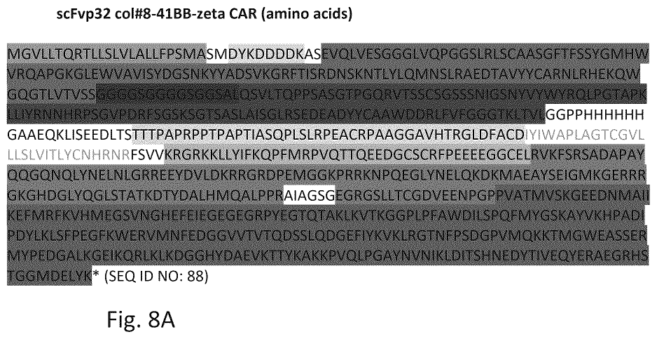

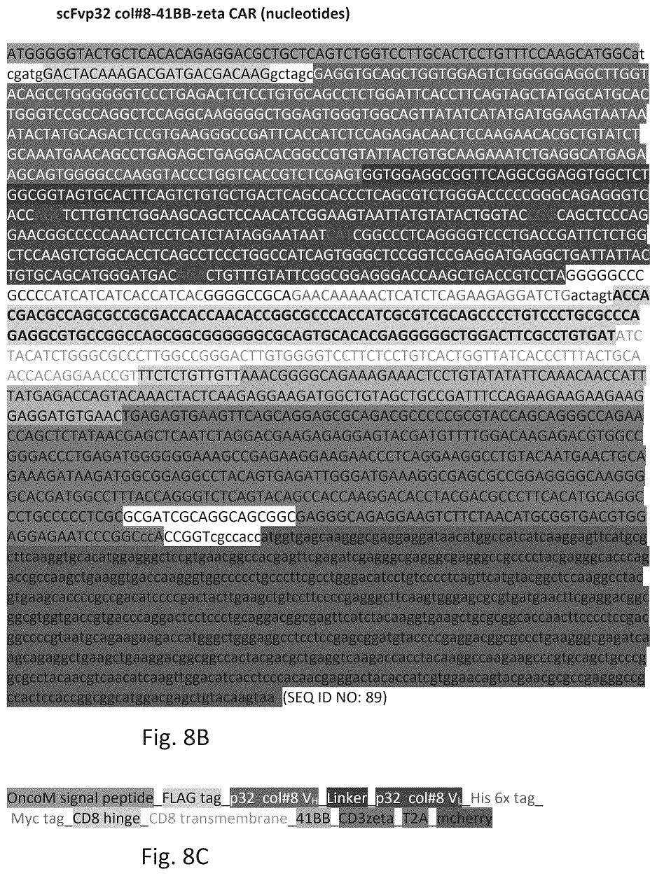

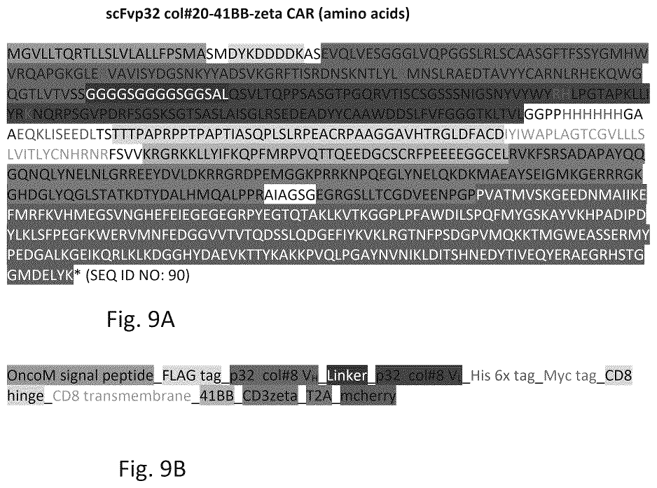

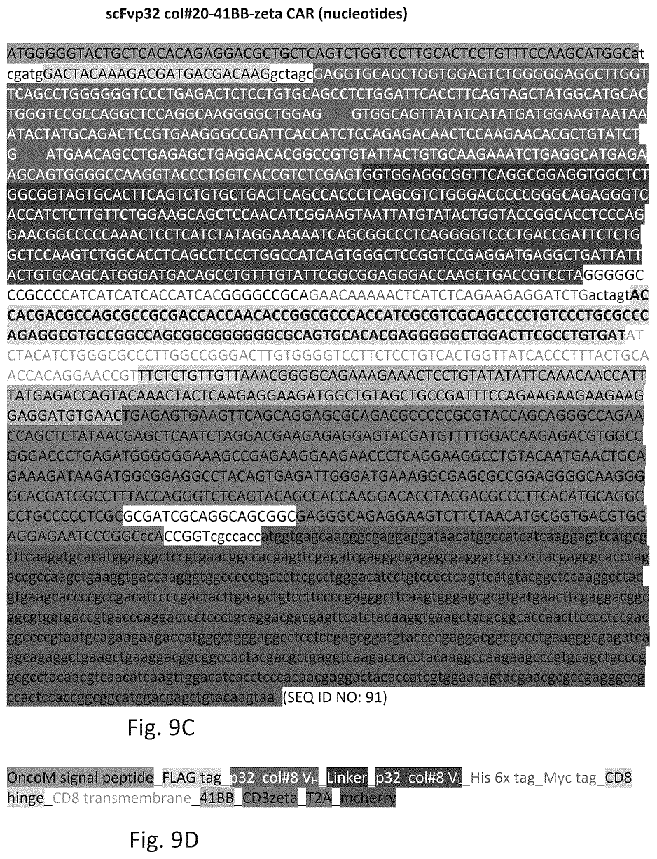

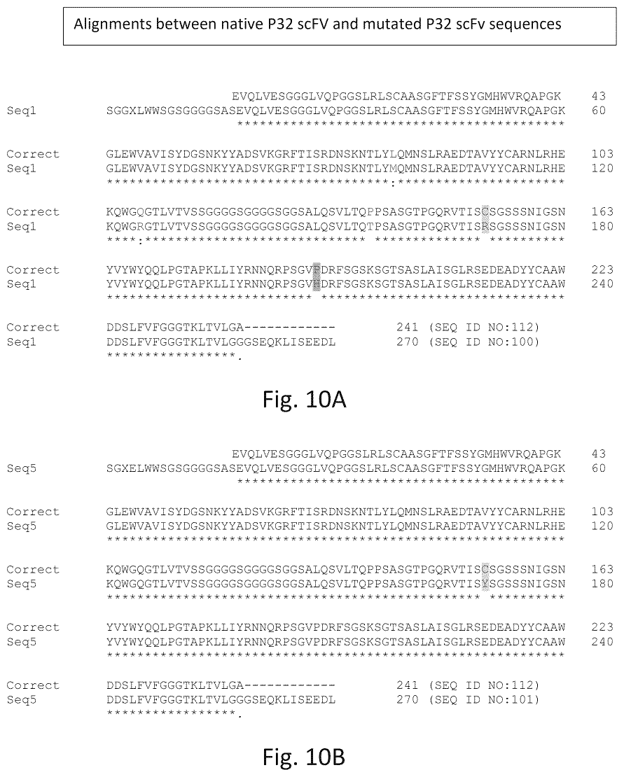

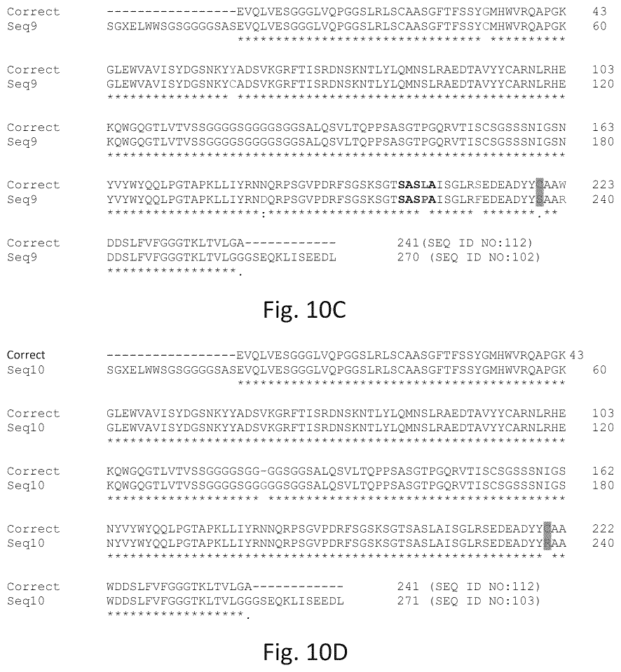

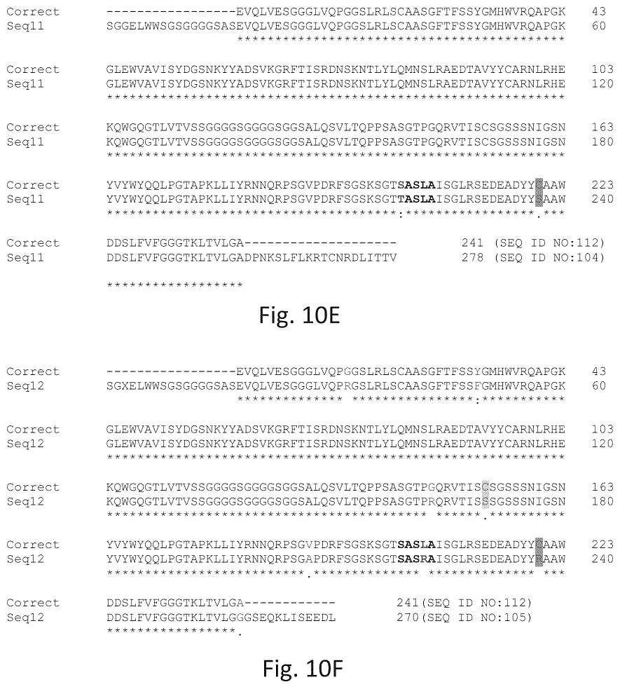

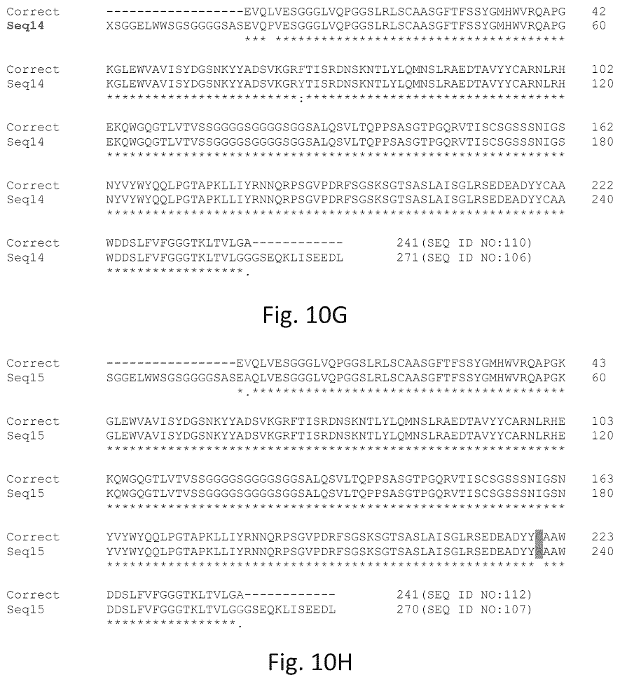

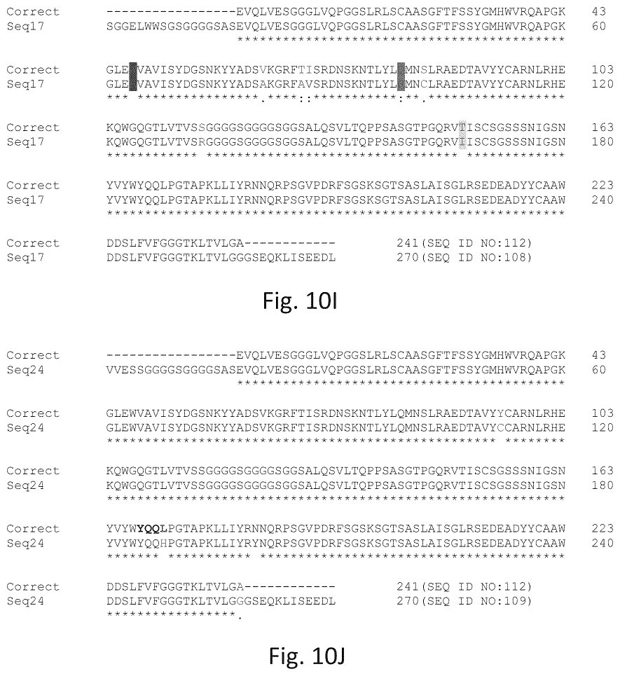

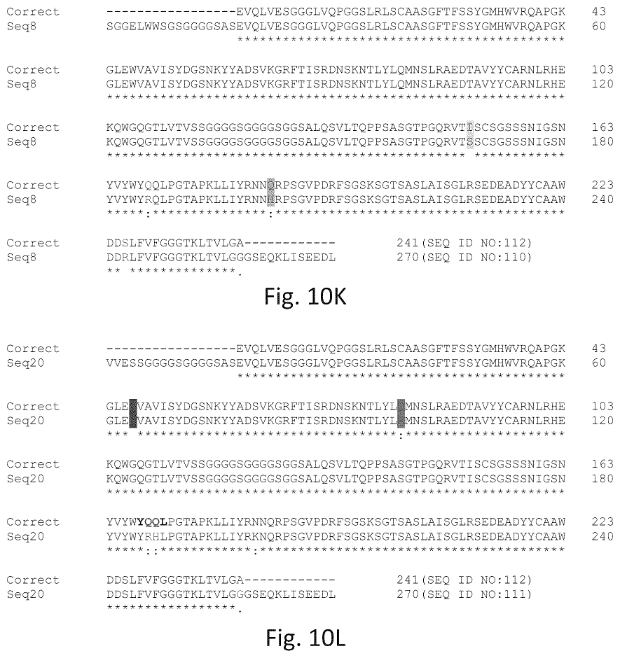

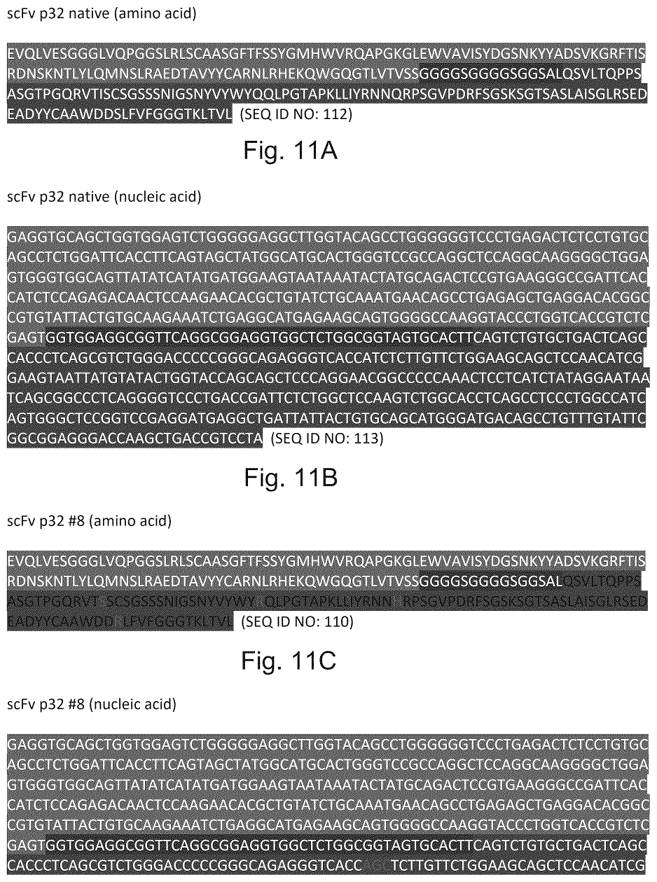

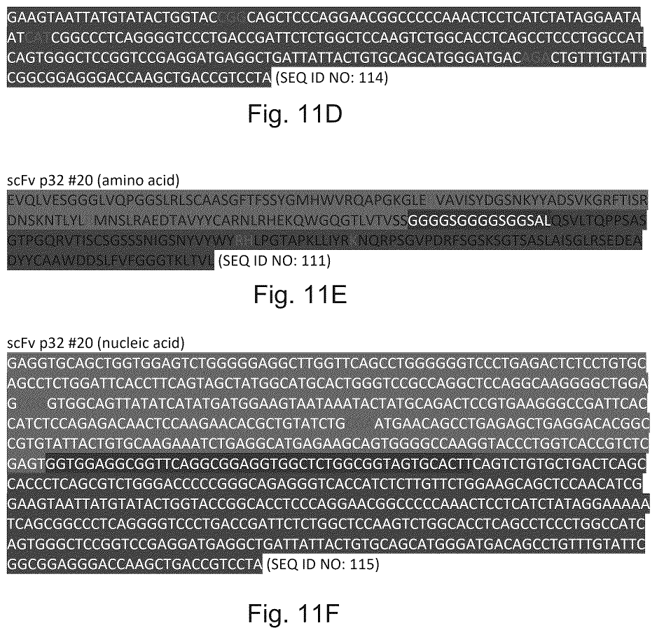

The present inventors generated p32 CAR T cells and found them to recognize and specifically eliminate murine and human p32 expressing glioma cells and tumor-derived endothelial cells in-vitro and to control tumor growth in orthotopic syngeneic and xenograft mouse models in a highly specific manner. The Examples section which follows shows that while the native p32 CAR T cells significantly reduced the percentage of residual tumor cells, clones #8 and #20 of p32 CAR T cells exhibit a significantly higher effect on reducing the percentage of residual tumor cells compared to the native p32 CAR T cells (FIGS. 3A-E). In addition, the present inventors showed that the p32-41BB-CD3zeta CAR T-cells have an improved function in terms of activation, proliferation and killing capability of tumor cells when compared to p32-CD28-FcRgamma CAR T-cells (FIGS. 4A-K). Moreover, CAR T cells, which were administered to the mice by intra-venous administration, and which comprise the p32 scFV reached the site of tumor (i.e., the brain) and bound therein to the p32 tumor-associated antigen (TAA). Furthermore, the CAR T cells were able to eliminate not only tumor cells and TDEC but also tumor associated macrophages (TAMs) (FIGS. 5A-I). Thus, the results presented herein clearly show the specificity of the p32+ macrophages to the tumor environment and their absence in the peripheral bone marrow.

The results presented herein demonstrate that p32 is selectively expressed on tumor-associated macrophages within the tumor microenvironment, as confirmed by flow cytometry and immunofluorescence. Importantly, no p32 expression was observed in CD68+ macrophages from healthy tissue or circulating bone marrow macrophages, suggesting that p32 expression is tumor-specific and localized to TAMs. Treatment with p32-CAR T cells significantly reduced the presence of TAMs in tumors, highlighting their potential to reprogram the immunosuppressive tumor microenvironment. These findings suggest that p32-positive TAMs are a novel, tumor-restricted target for p32-CAR T cells, providing a promising therapeutic approach with minimal off-target effects on healthy macrophages.

Since in gliomas p32 is expressed on tumor cells as well as in tumor-derived endothelial cells and tumor vasculature a p32 CAR T cell can be used as an anti-angiogenic agent.

According to an aspect of some embodiments of the present invention there is provided an isolated antibody or a fragment thereof which binds to a human p32 tumor associated antigen (TAA), the antibody comprising a heavy chain variable domain (VH) and a light chain variable domain (VL) each comprising three complementarity-determining regions (CDRs), wherein
(i) the VH CDRs 1, 2 and 3 comprise amino acid sequences SEQ ID NOs: 23, 25 and 27, respectively, and the VL CDRs 1, 2 and 3 comprise amino acid sequences SEQ ID NOs: 30, 32 and 34, respectively;
(ii) the VH CDRs 1, 2 and 3 comprise amino acid sequences SEQ ID NOs: 37, 39 and 41, respectively, and the VL CDRs 1, 2 and 3 comprise amino acid sequences SEQ ID NOs: 44, 46 and 48, respectively;
(iii) the VH CDRs 1, 2 and 3 comprise amino acid sequences SEQ ID NOs: 117, 119 and 121, respectively, and the VL CDRs 1, 2 and 3 comprise amino acid sequences SEQ ID NOs: 124, 126 and 128, respectively;
(iv) the VH CDRs 1, 2 and 3 comprise amino acid sequences SEQ ID NOs: 132, 134 and 136, respectively, and the VL CDRs 1, 2 and 3 comprise amino acid sequences SEQ ID NOs: 139, 141, 143, respectively;
(v) the VH CDRs 1, 2 and 3 comprise amino acid sequences SEQ ID NOs: 147, 149, 151, respectively, and the VL CDRs 1, 2 and 3 comprise amino acid sequences SEQ ID NOs: 154, 156 and 158, respectively;
(vi) the VH CDRs 1, 2 and 3 comprise amino acid sequences SEQ ID NOs: 145, 161 and 163, respectively, and the VL CDRs 1, 2 and 3 comprise amino acid sequences SEQ ID NOs: 166, 168, 170, respectively;
(vii) the VH CDRs 1, 2 and 3 comprise amino acid sequences SEQ ID NOs: 173, 175 and 177 respectively, and the VL CDRs 1, 2 and 3 comprise amino acid sequences SEQ ID NOs: 180, 182 and 184, respectively;
(viii) the VH CDRs 1, 2 and 3 comprise amino acid sequences SEQ ID NOs: 187, 189 and 191, respectively, and the VL CDRs 1, 2 and 3 comprise amino acid sequences SEQ ID NOs: 194, 196 and 198, respectively;
(ix) the VH CDRs 1, 2 and 3 comprise amino acid sequences SEQ ID NOs: 201, 203 and 205, respectively, and the VL CDRs 1, 2 and 3 comprise amino acid sequences SEQ ID NOs: 208, 210 and 212, respectively;
(x) the VH CDRs 1, 2 and 3 comprise amino acid sequences SEQ ID NOs: 215, 217 and 219, respectively, and the VL CDRs 1, 2 and 3 comprise amino acid sequences SEQ ID NOs: 222, 224 and 226, respectively;
(xi) the VH CDRs 1, 2 and 3 comprise amino acid sequences SEQ ID NOs: 229, 231 and 233, respectively, and the VL CDRs 1, 2 and 3 comprise amino acid sequences SEQ ID NOs: 236, 238 and 240, respectively; or
(xii) the VH CDRs 1, 2 and 3 comprise amino acid sequences SEQ ID NOs: 243, 245 and 247, respectively, and the VL CDRs 1, 2 and 3 comprise amino acid sequences SEQ ID NOs: 250, 252 and 254, respectively.

As used herein the term “p32” refers to the tumor associated antigen (TAA) p32 also known as gC1qR, HABP, C1qBP which is expressed on a surface of a cell in the tumor microenvironment.

The term “tumor microenvironment” or “TME” refers to the area surrounding the tumor.

In brain tumors, the tumor microenvironment typically comprises blood vessels, immune cells, neuronal cells, glial cells of the CNS, signaling molecules, and the extracellular matrix. The glial cells of the CNS, which are part of the TME, may include microglial cells, astrocytes, and oligodendrocytes.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)2 fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

The CDRs depicted herein were identified according to Kabat et al. (See, e.g., Kabat et al., 1992, Sequences of Proteins of Immunological Interest, 5th ed., Public Health Service, NIH, Washington D.C.).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule;
(iii) disulfide-stabilized Fv (“dsFv”), a genetically engineered antibody including the variable region of the light chain and the variable region of the heavy chain, linked by a genetically engineered disulfide bond;
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing and using antibodies or antibodies fragments according to some embodiments of the invention are provided herein-under.

As used herein the term “isolated” refers to at least partially separated from the natural environment.

For example, an isolated antibody can be at least partially separated from the organism producing same, e.g., a human body, or an animal body.

According to some embodiments the isolated antibody or the fragment thereof, is soluble.

According to some embodiments the isolated antibody is a recombinant molecule which does not exist in nature.

According to some embodiments the isolated antibody is a chimeric molecule which does not exist in nature.

According to some embodiments of the invention, the isolated antibody or the fragment thereof being insoluble.

According to some embodiments of the invention, the isolated antibody of
(i) further comprises heavy chain variable domain framework regions (FRs) 1, 2, 3 and 4 comprising amino acids SEQ ID NOs: 22, 24, 26 and 28, respectively;
(ii) further comprises heavy chain variable domain FRs 1, 2, 3 and 4 comprising amino acids SEQ ID NOs: 36, 38, 40 and 42, respectively;
(iii) further comprises heavy chain variable domain FRs 1, 2, 3 and 4 comprising amino acids SEQ ID NOs: 116, 118, 120 and 122, respectively;
(iv) further comprises heavy chain variable domain FRs 1, 2, 3 and 4 comprising amino acids SEQ ID NOs: 131, 133, 135 and 137, respectively;
(v) further comprises heavy chain variable domain FRs 1, 2, 3 and 4 comprising amino acids SEQ ID NOs: 146, 148, 150 and 152, respectively;
(vi) further comprises heavy chain variable domain FRs 1, 2, 3 and 4 comprising amino acids SEQ ID NOs: 130, 160, 162 and 164, respectively;
(vii) further comprises heavy chain variable domain FRs 1, 2, 3 and 4 comprising amino acids SEQ ID NOs: 172, 174, 176 and 178, respectively;
(viii) further comprises heavy chain variable domain FRs 1, 2, 3 and 4 comprising amino acids SEQ ID NOs: 186, 188, 190 and 192, respectively;
(ix) further comprises heavy chain variable domain FRs 1, 2, 3 and 4 comprising amino acids SEQ ID NOs: 200, 202, 204 and 206, respectively;
(x) further comprises heavy chain variable domain FRs 1, 2, 3 and 4 comprising amino acids SEQ ID NOs: 214, 216, 218 and 220, respectively;
(xi) further comprises heavy chain variable domain FRs 1, 2, 3 and 4 comprising amino acids SEQ ID NOs: 228, 230, 232 and 234, respectively; or
(xii) further comprises heavy chain variable domain FRs 1, 2, 3 and 4 comprising amino acids SEQ ID NOs: 242, 244, 246 and 248, respectively.

According to some embodiments of the invention, the antibody of
(i) further comprises light chain variable domain framework regions (FRs) 1, 2, 3 and 4 comprising amino acids SEQ ID NOs: 29, 31, 33 and 35, respectively;
(ii) further comprises light chain variable domain FRs 1, 2, 3 and 4 comprising amino acids SEQ ID NOs: 43, 45, 47 and 49, respectively;
(iii) further comprises light chain variable domain FRs 1, 2, 3 and 4 comprising amino acids SEQ ID NOs: 123, 125, 127 and 129, respectively;
(iv) further comprises light chain variable domain FRs 1, 2, 3 and 4 comprising amino acids SEQ ID NOs: 138, 140, 142 and 144, respectively;
(v) further comprises light chain variable domain FRs 1, 2, 3 and 4 comprising amino acids SEQ ID NOs: 153, 155, 157 and 159, respectively;
(vi) further comprises light chain variable domain FRs 1, 2, 3 and 4 comprising amino acids SEQ ID NOs: 165, 167, 169 and 171, respectively;
(vii) further comprises light chain variable domain FRs 1, 2, 3 and 4 comprising amino acids SEQ ID NOs: 179, 181, 183, 185, respectively;
(viii) further comprises light chain variable domain FRs 1, 2, 3 and 4 comprising amino acids SEQ ID NOs: 193, 195, 197 and 199, respectively;
(ix) further comprises light chain variable domain FRs 1, 2, 3 and 4 comprising amino acids SEQ ID NOs: 207, 209, 211 and 213, respectively;
(x) further comprises light chain variable domain FRs 1, 2, 3 and 4 comprising amino acids acids SEQ ID NOs: 221, 223, 225 and 227, respectively;
(xi) further comprises light chain variable domain FRs 1, 2, 3 and 4 comprising amino acids SEQ ID NOs: 235, 237, 239 and 241, respectively; or
(xii) further comprises light chain variable domain FRs 1, 2, 3 and 4 comprising amino acids SEQ ID NOs: 249, 251, 253 and 255, respectively.

According to some embodiments of the invention, the isolated antibody or the fragment thereof comprises a heavy chain variable domain comprising an amino acid sequence having at least 90% sequence identity e.g., at least 91%, 92%, 93%, 94%, 95%, 96%, 97%, 98%, 99% sequence identity or more to the amino acid sequence selected from the group consisting of SEQ ID NOs: 92, 96, 256, 258, 260, 262, 264, 266, 268, 270, 272 and 274.

According to some embodiments of the invention, the isolated antibody or the fragment thereof comprises a heavy chain variable domain comprising the amino acid sequence selected from the group consisting of SEQ ID NOs: 92, 96, 256, 258, 260, 262, 264, 266, 268, 270, 272 and 274.

According to some embodiments of the invention, the isolated antibody or the fragment thereof comprises a light chain variable domain comprising an amino acid sequence at least 90% sequence identity, e.g., at least 91%, 92%, 93%, 94%, 95%, 96%, 97%, 98%, 99% sequence identity or more to the amino acid sequence selected from the group consisting of SEQ ID NOS: 93, 97, 257, 259, 261, 263, 265, 267, 269, 271, 273 and 275.

According to some embodiments of the invention, the isolated antibody or the fragment thereof comprises a light chain variable domain comprising the amino acid sequence selected from the group consisting of SEQ ID NOs: 93, 97, 257, 259, 261, 263, 265, 267, 269, 271, 273 and 275.

Tools for determining and qualifying identities between 2 or more sequences are provided hereinbelow.

According to some embodiments of the invention, the isolated antibody or the fragment thereof being a single chain Fv (scFv).

According to some embodiments of the invention, the scFv is selected from the group consisting of SEQ ID NOs: 110, 111 and 100-109.

According to some embodiments of the invention, the isolated antibody or the fragment thereof being conjugated to a functional moiety.

According to an aspect of some embodiments of the invention, there is provided a molecule comprising the antibody of some embodiments of the invention being conjugated to a functional moiety (also referred to as an “immuneconjugate”) such as a detectable or a therapeutic moiety. The immunoconjugate molecule can be an isolated molecule such as a soluble and/or a synthetic molecule.

According to some embodiments of the invention, the functional moiety is a therapeutic moiety.

The immunoconjugate can include the antibody of some embodiments of the invention and a therapeutic moiety such as a drug, a prodrug, an immune activation molecule, a cytotoxic moiety, a toxic moiety, a cytokine moiety and/or a second antibody moiety comprising a different specificity to the antibodies of the invention.

In an antibody-directed enzyme prodrug therapy (ADEPT) an antibody targets a tumor antigen and is conjugated with an enzyme. The enzyme then activates a non-toxic prodrug into a cytotoxic drug at the tumor site, reducing damage to healthy tissues. Non-limiting example of enzymes which can be used include, but are not limited to, β-glucuronidase, which converts prodrugs into active chemotherapeutics; Carboxypeptidase G2, which activates nitrogen mustard-based prodrugs; and alkaline phosphatase, which activates phosphate-based prodrugs.

In an antibody-drug conjugate (ADC), enzymes can be part of the payload mechanism, ensuring precise drug activation within cancer cells. For example, Brentuximab vedotin (Adcetris) is an antibody-drug conjugate (ADC) designed to target and kill cancer cells expressing CD30, a protein found on the surface of certain lymphomas, including Hodgkin lymphoma and anaplastic large cell lymphoma; and Trastuzumab emtansine (Kadcyla) is an ADC designed to target and kill cancer cells overexpressing HER2 (human epidermal growth factor receptor 2), common in some breast cancers.

Enzyme-fused antibodies for immune modulation use enzymes to amplify immune responses at the tumor site. Examples include, but are not limited to, Pegylated asparaginase (which depletes asparagine, starving cancer cells), IDO (Indoleamine 2,3-dioxygenase) inhibitors (which modify immune checkpoints).

Additional or alternative therapeutic moieties which can be conjugated to the antibody of the invention are provided in Table 1, hereinbelow.

TABLE 1

| | Amino acid sequence | Nucleic acid sequence |
|---|---|---|
| Therapeutic | (GenBank Accession No.)/ | (GenBank Accession No.)/ |
| moiety | SEQ ID NO: | SEQ ID NO: |
| Pseudomonas | ABU63124/276 | EU090068/277 |
| exotoxin | | |
| Diphtheria | AAV70486/278 | AY820132.1/279 |
| toxin | | |
| interleukin 2 | CAA00227/280 | A02159/281 |
| CD3 | P07766/282 | X03884/283 |
| CD16 | NP_000560.5/284 | NM_000569.6/285 |
| interleukin 4 | NP_000580.1/286 | NM_000589.2/287 |
| HLA-A2 | P01892/288 | K02883/289 |
| interleukin 10 | P22301/290 | M57627/291 |
| Ricin toxin | EEF27734/292 | EQ975183/293 |

According to some embodiments of the invention, the functional moiety is a detectable moiety.

Various types of detectable or reporter moieties may be conjugated to the antibody of the invention. These include, but not are limited to, a radioactive isotope (such as [¹²⁵]iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, U K. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimmer or tetramer form of the antibody).

Table 2 provides non-limiting examples of identifiable moieties which can be conjugated to the antibody of the invention.

TABLE 2

| | Amino Acid sequence | Nucleic Acid sequence |
|---|---|---|
| | (GenBank Accession | (GenBank Accession |
| Identifiable Moiety | No.)/SEQ ID NO: | No.)/SEQ ID NO: |
| Green Fluorescent | AAL33912/294 | AF435427/295 |
| protein | | |
| Alkaline phosphatase | AAK73766/296 | AY042185/297 |
| Peroxidase | CAA00083/298 | A00740/299 |
| Histidine tag | Amino acids 264-269 of | Nucleotides 790-807 of |
| | GenBank Accession No. | GenBank Accession No. |
| | AAK09208/300 | AF329457/301 |
| Myc tag | Amino acids 273-283 of | Nucleotides 817-849 of |
| | GenBank Accession No. | GenBank Accession No. |
| | AAK09208/302 | AF329457/303 |
| Biotin lygase tag | LHHILDAQKMVWNHR/ | |
| | 304 | |
| orange fluorescent | AAL33917/305 | AF435432/306 |
| protein | | |
| Beta galactosidase | ACH42114/307 | EU626139/308 |
| Streptavidin | AAM49066/309 | AF283893/310 |

The functional moiety (the detectable or therapeutic moiety of some embodiments of the invention) may be attached or conjugated to the antibody or antibody fragment of some embodiments of the invention in various ways, depending on the context, application and purpose.

When the functional moiety is a polypeptide, the immunoconjugate may be produced by recombinant means. For example, the nucleic acid sequence encoding a toxin or a fluorescent protein [e.g., green fluorescent protein (GFP), red fluorescent protein (RFP) or yellow fluorescent protein (YFP)] may be ligated in-frame with the nucleic acid sequence encoding the antibody or the fragment thereof of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., www (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties to the antibody of the invention include for example, SPDP conjugation (e.g., as described in Cumber et al. (1985, Methods of Enzymology 112:207-224); Glutaraldehyde conjugation (e.g., which is described in G. T. Hermanson (1996, “Antibody Modification and Conjugation, in Bioconjugate Techniques, Academic Press, San Diego)); Carbodiimide conjugation (e.g., using a dehydrating agent such as a carbodiimide, e.g., in the presence of 4-dimethyl aminopyridine (e.g., described in J. March, Advanced Organic Chemistry: Reaction's, Mechanism, and Structure, pp. 349-50 & 372-74 (3d ed.), 1985; B. Neises et al. (1978); Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)), each of which is fully incorporated herein by reference.

According to an aspect of some embodiments of the invention, there is provided a chimeric antigen receptor (CAR) comprising the p32 antibody fragment of some embodiments of the invention and an intracellular signaling domain, the intracellular signaling domain comprising a co-stimulatory signaling region and an activation domain.

As used herein the phrase “chimeric antigen receptor (CAR)” refers to a synthetic receptor engineered to direct immune cells, such as T cells, to specifically recognize cells expressing p32. The CAR comprises a sequence of an antibody or a fragment thereof and an intracellular signaling module which brings about a signaling activity once the antigen is bound to the antibody.

According to some embodiments of the invention, the CAR comprising the p32 antibody fragment of some embodiments of the invention is capable of killing cancer cells.

According to some embodiments of the invention, the co-stimulatory signaling region comprises CD28, 41BB, OX40, ICOS, CD27, MYD88-CD40, Toll-like receptor (TLRs) or KIR2DS2.

The term “41BB” or “4-1BB” which is interchangeably used herein refers to the CD137 (TNFRS9) inducible costimulatory receptor.

The term “CD28” (also known as Tp44; IMD123) as used herein refers to a protein essential for T-cell proliferation and survival, cytokine production, and T-helper type-2 development.

The term “OX40” (also known as “TNF receptor superfamily member 4”, ACT35, CD134, IMD16, TXGP1L) as used herein refers to a member of the TNF-receptor superfamily which has been shown to activate NF-kappaB through its interaction with adaptor proteins TRAF2 and TRAF5.

The term “ICOS” (inducible T cell costimulatory; also known as AILIM, CD278, CVID1) as used herein refers to a protein which belongs to the CD28 and CTLA-4 cell-surface receptor family.

The term “CD27” (also known as T14; S152; Tp55; TNFRSF7; S152. LPFS2) as used herein refers to a member of the TNF-receptor superfamily. The CD27 receptor is required for generation and long-term maintenance of T cell immunity.

The term “MYD88” (MYD88 innate immune signal transduction adaptor; also known as WM1; IMD68; MYD88D) as used herein refers to cytosolic adapter protein that plays a central role in the innate and adaptive immune response. This protein functions as an essential signal transducer in the interleukin-1 and Toll-like receptor signaling pathways.

The term “CD40” (also known as p50; Bp50; CDW40; TNFRSF5) as used herein refers to member of the TNF-receptor superfamily. The encoded protein is a receptor on antigen-presenting cells of the immune system and is essential for mediating a broad variety of immune and inflammatory responses including T cell-dependent immunoglobulin class switching, memory B cell development, and germinal center formation.

The term “KIR2DS2” (killer cell immunoglobulin like receptor, two Ig domains and short cytoplasmic tail 2) as used herein refers to a member of the killer-cell immunoglobulin-like receptor (KIR) family, specifically a type of activating receptor found on the surface of natural killer (NK) cells and some T cells.

According to some embodiments of the invention, the activation domain comprises FcRgamma or CD3zeta.

The term “FcRgamma” (also known as FCER1G, Fc epsilon receptor Ig) as used herein refers to a protein which is part of the high affinity IgE receptor, a key molecule involved in allergic reactions. The receptor is a tetramer composed of 1 alpha, 1 beta, and 2 gamma chains. The gamma chains are also subunits of other Fc receptors.

The term “CD3zeta” (also known as CD247, T3Z, CD3H, CD3Q, CD3Z, TCRZ, IMD25) as used herein refers to a T-cell receptor zeta, which together with T-cell receptor alpha/beta and gamma/delta heterodimers, and with CD3-gamma, -delta and -epsilon, forms the T-cell receptor-CD3 complex. The zeta chain plays an important role in coupling antigen recognition to several intracellular signal-transduction pathways.

According to some embodiments of the invention, the CAR of some embodiments of the invention comprising the amino acid sequence as set forth by SEQ ID NO: 88 or 90.

According to an aspect of some embodiments of the invention, there is provided a chimeric antigen receptor (CAR) comprising a p32 antibody fragment and an intracellular signaling domain, the intracellular signaling domain comprising a co-stimulatory signaling region and an activation domain, wherein the co-stimulatory signaling region comprises the 41BB amino acid sequence and the activation domain comprises the CD3zeta amino acid sequence.

According to some embodiments of the invention, the p32 antibody fragment, which is included in the CAR of some embodiments of the invention, comprises a heavy chain CDRs 1, 2 and 3 as set forth by SEQ ID NOs: 9, 11, and 13, respectively.

According to some embodiments of the invention, the p32 antibody fragment, which is included in the CAR of some embodiments of the invention, comprises light chain CDRs 1, 2 and 3 as set forth by SEQ ID NOs: 16, 18 and 20, respectively.

According to some embodiments of the invention, the CAR of some embodiments of the invention, further comprises heavy chain variable domain comprising framework regions (FRs) 1, 2, 3 and 4 as set forth by SEQ ID NOs: 8, 10, 12, and 14, respectively.

According to some embodiments of the invention, the CAR of some embodiments of the invention, further comprises light chain variable domain comprising framework regions (FRs) 1, 2, 3 and 4 as set forth by SEQ ID NOs: 15, 17, 19 and 21, respectively.

According to some embodiments of the invention, the CAR of some embodiments of the invention, comprising the amino acid sequence as set forth by SEQ ID NO: 82.

According to some embodiments of the invention, the CAR of some embodiments of the invention further comprises a hinge sequence, a transmembrane domain and/or a cell surface or secreted immunomodulatory molecule.

As used herein the phrase “hinge sequence” refers to a flexible linker between the antibody fragment of some embodiments of the invention and the transmembrane domain of the CAR of some embodiments of the invention, which allows the antibody fragment to be positioned at an optimal distance from the cell membrane so as to enable an effective interaction between the antibody and the antigen on target cell.

According to some embodiments of the invention, the hinge sequence comprises CD8 hinge, CD28 hinge, IgG1 hinge or IgG4 hinge sequence.

According to some embodiments of the invention, the transmembrane domain (TMD) comprises a CD3-zeta TMD, CD8alpha TMD, CD4 TMD, CD28 TMD or ICOS TMD.

According to some embodiments of the invention, the cell surface or secreted immunomodulatory molecule enhances T cell function or modifies tumor microenvironment.

According to some embodiments of the invention, the CAR of some embodiments of the invention, being soluble.

According to some embodiments of the invention, the CAR of some embodiments of the invention, being insoluble.

According to an aspect of some embodiments of the invention, there is provided a nucleic acid construct comprising a polynucleotide comprising a nucleic acid sequence encoding the isolated antibody or the fragment thereof of some embodiments of the invention, wherein the polynucleotide is operably linked to a promoter for directing expression of the nucleic acid sequence in a host cell.

According to an aspect of some embodiments of the invention, there is provided a nucleic acid construct comprising a polynucleotide comprising a nucleic acid sequence encoding the CAR of some embodiments of the invention, wherein the polynucleotide is operably linked to a promoter for directing expression of the nucleic acid sequence in a host cell.

The nucleic acid construct of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

A non-limiting example of a promoter suitable for expression of the CAR construct of some embodiments of the invention is the EF1-alpha (elongation factor 1 alpha) promoter.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation of the polypeptide of interest (e.g., the antibody or the antibody fragment thereof of some embodiments of the invention). Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the polypeptide of interest can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149:51-60).

Recombinant viral vectors are useful for in vivo expression of the polypeptide of interest since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into a host cell of interest. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, transformation, lipofection, electroporation, infection with recombinant viral vectors, or transduction with viruses. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). Phage or viral vectors can be introduced into host cells, after growth of infectious particles in suitable packaging cells, many of which are commercially available. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the polypeptide of interest of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the polypeptide of interest and the heterologous protein, the polypeptide of interest can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by some embodiments of the invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to some embodiments of the invention, the host cell is genetically modified to express the nucleic acid construct of some embodiments of the invention, e.g., the CAR construct of some embodiments of the invention.

According to some embodiments of the invention, the host cell is an immune cell.

According to an aspect of some embodiments of the invention, there is provided an isolated genetically modified immune cell which expresses the CAR of some embodiments of the invention, or the antibody or the fragment thereof of some embodiments of the invention.

According to some embodiments of the invention, the immune cell is a T lymphocyte cell.

According to some embodiments of the invention, the immune cell is a natural killer (NK) cell, natural killer T cell (NKT cells), macrophage, gamma delta (78) T cell, dendritic cell, or B cell.

According to some embodiments of the invention, the immune cell is a NK cell, NKT cell, or B cell.

According to some embodiments of the invention, the immune cell is a natural killer T cell.

According to some embodiments of the invention, the immune cell is a natural killer cell.

According to some embodiments of the invention, the antibody is expressed as a membrane bound polypeptide or transmembrane polypeptide of the host cell.

According to some embodiments of the invention, the antibody is secreted from the host cell.

According to some embodiments of the invention, the antibody is expressed as a transmembrane polypeptide of the host cell.

According to some embodiments of the invention, the genetically modified immune cell is adoptively transferred to the subject in need thereof.

According to some embodiments of the invention, the CAR of some embodiments of the invention is expressed as a transmembrane protein within the host cell.

The isolated antibody or the fragment thereof of some embodiments of the invention, the nucleic acid construct of some embodiments of the invention, or the isolated genetically modified immune cell (e.g., T cell) of some embodiments of the invention can be administered to an organism per se, or in a pharmaceutical composition where it is mixed with suitable carriers or excipients.

According to an aspect of some embodiments of the invention, there is provided a pharmaceutical composition comprising the isolated antibody or the fragment thereof of some embodiments of the invention, the nucleic acid construct of some embodiments of the invention, or the isolated genetically modified immune cell (e.g., T cell) of some embodiments of the invention, and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the isolated antibody or the fragment thereof of some embodiments of the invention, the nucleic acid construct of some embodiments of the invention, or the isolated genetically modified immune cell (e.g., T cell) of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (the isolated antibody or the fragment thereof of some embodiments of the invention, the nucleic acid construct of some embodiments of the invention, or the isolated genetically modified immune cell (e.g., T cell) of some embodiments of the invention) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to an aspect of some embodiments of the invention, there is provided the isolated antibody or the fragment thereof of some embodiments of the invention, the nucleic acid construct of some embodiments of the invention, or the isolated genetically modified immune cell (e.g., T cell) of some embodiments of the invention, for use in the treatment of a p32-associated cancer.

According to an aspect of some embodiments of the invention, there is provided a method of treating a subject diagnosed with a p32-associated cancer, the method comprising administering to the subject a therapeutically effective amount of isolated antibody or the fragment thereof of some embodiments of the invention, the nucleic acid construct of some embodiments of the invention, or the isolated genetically modified immune cell (e.g., T cell) of some embodiments of the invention, thereby treating the subject diagnosed with a p32-associated cancer.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the phrase “p32-associated cancer” refers to any cancer characterized by expression of the p32 TAA on the cell surface of the cancerous cell.

It should be noted that the p32 TAA can be expressed on the surface of a tumor cell, a macrophage residing in the tumor microenvironment, or a tumor-derived endothelial cell, and the agents described herein can be used to inhibit expression of the p32 on the tumor cell, on the macrophage residing in the tumor microenvironment and/or on the tumor-derived endothelial cell.

As used herein “tumor-derived endothelial cell” (TDEC) refers to an endothelial cell which has been differentiated and formed from a tumor stem cell, and has the ability to form blood vessels which support the growth of the tumor.

It should be noted that since the p32 TAA is not expressed on a circulating macrophage (i.e., on a macrophage which is not derived from the tumor microenvironment), the use of the claimed agents (e.g., the isolated antibodies or fragments thereof, the CAR, the constructs and/or the T cells of some embodiments of the invention) is specific to the tumor microenvironment and is not expected to cause unspecific effect.

According to some embodiments of the invention, the p32-associated cancer is a solid tumor.

According to some embodiments of the invention, the p32-associated cancer is glioblastoma, breast cancer, lung cancer, thyroid cancer, ovary cancer, skin cancer, pancreatic cancer or testicular cancer.

According to an aspect of some embodiments of the invention, there is provided a method of inhibiting angiogenesis in a p32-associated cancer, comprising contacting cells of a p32-associated cancer tumor microenvironment (TME) with the isolated antibody or the fragment thereof of some embodiments of the invention, the nucleic acid construct of some embodiments of the invention, or the isolated genetically modified immune cell (e.g., T cell) of some embodiments of the invention, thereby inhibiting angiogenesis in the p32-associated cancerous tumor.

According to some embodiments of the invention the contacting is effected in-vivo by administration of the T cells into a subject.

According to some embodiments of the invention the contacting is effected in-vitro.

For example, including for example, adding the active agent (e.g., isolated antibody or the fragment thereof of some embodiments of the invention, the nucleic acid construct of some embodiments of the invention, or the isolated genetically modified immune cell (e.g., T cell) of some embodiments of the invention) to the cells of a p32-associated cancer tumor microenvironment (TME) such that the active agent is in direct contact with the cells.

According to some embodiments of the invention, the cells of the p32-associated cancer tumor microenvironment are incubated with the active agent of some embodiments of the invention.

According to some embodiments of the invention, the cells of the p32-associated cancer TME are endothelial cells.

According to some embodiments of the invention, the endothelial cells are tumor-derived endothelial cells.

According to an aspect of some embodiments of the invention, there is provided a method of inhibiting expression of p32 on a tumor cell, a macrophage residing in a tumor microenvironment or a tumor-derived endothelial cell, contacting the tumor cell, the macrophage or the tumor-derived endothelial cell with the isolated antibody or the fragment thereof of some embodiments of the invention, the nucleic acid construct of some embodiments of the invention, or the isolated genetically modified immune cell (e.g., T cell) of some embodiments of the invention, thereby inhibiting expression of the p32 on the tumor cell, the macrophage residing in the tumor microenvironment or the tumor-derived endothelial cell.

According to some embodiments of the invention, the contacting the tumor cell, the macrophage or the tumor-derived endothelial cell is effected in-vivo.

According to some embodiments of the invention, the contacting the tumor cell, the macrophage or the tumor-derived endothelial cell is effected in-vitro.

### Methods of Preparing and Using Antibodies

Following is a non-limiting description of methods of producing and using antibodies or antibodies fragments according to some embodiments of the invention.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U.S.A 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U.S.A 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73:119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2:97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2:106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F (ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147 (1): 86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368:856-859 (1994); Morrison, Nature 368 812-13 (1994); Fishwild et al., Nature Biotechnology 14, 845-51 (1996); Neuberger, Nature Biotechnology 14:826 (1996); and Lonberg and Huszar, Intern. Rev. Immunol. 13, 65-93 (1995).

It will be appreciated that targeting of particular compartment within the cell can be achieved using intracellular antibodies (also known as “intrabodies”). These are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92:3137-3141; Deshane et al., 1994, Gene Ther. 1:332-337; Marasco et al., 1998 Human Gene Ther 9:1627-42; Shaheen et al., 1996 J. Virol. 70:3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12:396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Once antibodies are obtained, they may be tested for activity, for example via ELISA.

Following is a non-limiting description of preparing a genetically modified immune cells (e.g., immune cells which are transduced to express the CAR of some embodiments of the invention) and administering same to a subject in need thereof.

For production of a stable cell line which produces the CAR of some embodiments of the invention a retroviral packaging system is used, for example, the GAL-V RD114 system, which comprises the 293 Vec GAL-V and 293Vec RD114 cells. The 293Vec GAL-V cells are HEK293 cells engineered to express the gibbon ape leukemia virus (GAL-V) envelope protein. The 293 Vec RD114 cells are HEK293 cells engineered to express the RD114 envelope protein, derived from a feline endogenous retrovirus, which is useful for enhancing delivery intro hematopoietic cells, stem cells and lymphocytes.

Briefly, 293 Vec GAL-V cells are transfected with the CAR of some embodiments of the invention, and the supernatant containing the retroviral particles are collected at 48 hours. Viral media can be then filtered and Polyberen can be added to a final concentration of 8 μg/ml. Infection of 293 Vec RD114 cells is done by using the supernatant of the viral media, preferably centrifuging the cells with the media for 10 minutes with no breaks at room temperature, and followed by incubation (e.g., overnight). Following infection, 293Vec RD114 cells are sorted to select only the CAR expressing packaging cells.

Immune cells can be isolated from peripheral blood mononuclear cells (PBMCs) of a subject. For example, PBMCs are separated on a centrifuge gradient (e.g., on Lymphoprep; Ferenius Kabi Norge AS), and activated for 48 hours on non-tissue culture plates (e.g., 6-well plates) which are pre-coated with antiCD3 and antiCD28 antibodies (e.g., available from Biolegend). On day 2, activated T cells (e.g., at a concentration of 2-3×10⁶ cells/well, e.g., at a concentration of 2.5×10⁶ cells/well) are transduced with retroviral supernatant obtained from either CAR-PG13 or CAR-RD114 cells using “spin-infection” on RetroNectin 6-well coated plates with addition of 100 U/ml IL-2 (PeproTech).

Evaluation of transduction efficiency can be estimated by analyzing FLAG cell surface expression, as well as mcherry expression on retrovirally transduced cells and comparing to either isotype control stained or un-transduced T cells (UT).

The immune cells transduced to express the CAR of some embodiments of the invention can be derived from either autologous sources such as self bone marrow cells or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include for example, the use of gene-editing techniques (CRISPR/Cas9 editing) to prepare “universal” allogeneic CAR T cells products that avoid graft-versus-host disease (GVHD) and host-mediated graft rejection, essentially as described in Diorio, C., et al., 2024 (“Allogeneic chimeric antigen receptor cell therapies for cancer: progress made and remaining roadblocks”. Nat. Rev. Clin. Oncol. Hypertext Transfer Protocol Secure://doi(dot)org/10.1038/s41571-024-00959-y; which is fully incorporated herein by reference in its entirety).

As described above, administration of the immune cells transduced with the CAR of some embodiments of the invention to the subject in need thereof can be effected using any suitable route such as intravenous, intra kidney, intra gastrointestinal track, intra peritoneal administrations, intra-tumor, intra CNS methods (as described above), e.g., by intra ventricular routes.

### Sequence Identities

As used herein, “sequence identity” or “identity” in the context of two polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff JG. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN or the BlastP software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

Pairwise global alignment was defined by S. B. Needleman and C. D. Wunsch, (“A general method applicable to the search of similarities in the amino acid sequence of two proteins” Journal of Molecular Biology, 1970, pages 443-53, volume 48).

For example, when starting from a polypeptide sequence and comparing to other polypeptide sequences, the EMBOSS-6.0.1 Needleman-Wunsch algorithm (available from emboss(dot)sourceforge(dot)net/apps/cvs/emboss/apps/needle(dot)html) can be used to find the optimum alignment (including gaps) of two sequences along their entire length—a “Global alignment”. Default parameters for Needleman-Wunsch algorithm (EMBOSS-6.0.1) include: gapopen=10; gapextend=0.5; datafile=EBLOSUM62; brief=YES.

According to some embodiments of the invention, the parameters used with the EMBOSS-6.0.1 tool (for protein-protein comparison) include: gapopen=8; gapextend=2; datafile=EBLOSUM62; brief=YES.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 94 is expressed in a DNA sequence format (e.g., reciting T for thymine), but it can refer to either a DNA sequence that corresponds to a p32 scFv nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### General Materials and Experimental Methods

### Antibodies

The following antibodies were used in the experiments: Mouse anti c-myc (clone 9E10) (Santa Cruz Biotechnology), APC-Streptavidin (SouthernBiotech), Alexa Fluor 488 goat anti-mouse IgG1 (Life). Additional antibodies are described herein.

### Random Mutagenesis and Library Generation

Validation of Cell Surface Expression and Sequence of Native/WT scFV P32

P32 scFv was amplified from pCR3.1-2.15 plasmid by the following primers:

For:
(SEQ ID NO: 1)
GgtggaggcggtagcggaggcggagggtcggctagcG
AGGTGCAGCTGGTGGAGTCTG;
Rev:
(SEQ ID NO: 2)
TTACAAgtcctcttcagaaataagcttttgttcggatcc
gcccccTAGGACGGTCAGCTTGGTCCCTC

PETCON2 plasmid contains HA and cMyc tags to label the scFv: HA tag starts 30 amino acids upstream to VH; The c-myc tag starts 5 amino acids downstream to VL, and a GGGS linker (SEQ ID NO: 3) is present between VL to the c-myc tag. pETCON2 plasmid was digested with Ndel and BamHI (Fermentas). Digested vector were extracted from gel using Wizard SV GEL & PCR clean-up system (Promega).

Both p32 scFV (as insert) and the cut PETCON2 plasmid were transformed into EBY100 competent cells at 3:1 ratio, 10 μl of each 1 ml transformation mix, plated as a spot on YPD and SD-Trp plates. Yeast were then collected (13,000 g; 30 sec) and plated on SD-Trp by “semi-isolating” form. Selected colonies were evaluated for antigen binding by FACS analysis, plasmids were purified by Zymoprep yeast miniprep kit and their scFv sequence was confirmed.

### Generation of p32 Library and Mutagenesis

scFv was first amplified by mixing 2 μl scFv template, 1 μl of each primer, 25 μl 2× Phusion hot start flex 2× master mix (NEB) and 21 μl water. PCR settings were 95 °C for 2 minutes, followed by 30 cycles of 95 °C, for 30 seconds(s), 55 °C for 30 seconds, 72 °C for 60 seconds and final incubation of 72 °C for 5 minutes. Amplified fragment was purified and then subjected to mutagenesis by mixing 100 and 200 ng template, 1 μl of each primer, 1 μl dNTPs, 5 μl buffer, 1 μl polymerase of GeneMorph II Random mutagenesis Kit (Agilent) complete to 50 μl with water. Mutagenesis settings were 95 °C for 2 minutes followed by 17 cycles of 95 °C for 30 seconds, 60 °C for 30 seconds, 72 °C for 60 seconds and final incubation of 72 °C for 10 minutes. Each mutated fragment (100 and 200 ng original templates) was purified then amplified by preparing five identical reactions of 2 μl scFv template, 1 μl of each primer, 25 μl 2× Phusion hot start flex 2× master mix (NEB) and 21 μl water. PCR settings were 95 °C for 2 minutes followed by 30 cycles of 95 °C for 30 seconds 55 °C for 30 seconds, 72 °C for 60 seconds and final incubation of 72 °C for 10 minutes. Amplified mutated fragments were purified from agarose gel. To generate P32 library, yeast cells were prepared and electroporated according to Benatuil et al. 2010 (Protein Eng Des Sel. 2010; 23:155-159).

### Yeast FACS Staining and Sorting

Human p32 antigen (Sigma, PREST ANTIGEN C1QBP APREST75557) was biotinylated by EZ-Link Sulfo-NHS-SS-Biotin kit (Thermo) according to manufacturer instructions.

P32 yeast library was cultured in SD-Trp media at 30 °C, passaged for three days, then scFv was expressed by changing the media to SG-Trp and the temperature to 20 °C. For first panning 1×10⁷ yeast cells were washed with 1 ml PBS+0.5% ovalbumin (sigma) (assay buffer) then incubated with 100 ng/ml p32-Biotin and mouse anti-c-myc 1:50, both diluted in assay buffer for 1 hour in room temperature with rotation. Cells were washed with 1 ml ice cold assay buffer, then incubated for 40 minutes on ice with APC-Streptavidin and Alexa Fluor 488 goat anti-mouse IgG1 diluted 1:50 and 1:200 respectively in assay buffer. Cells were washed with 1 ml ice cold PBS, then top 5% of double positive yeast were sorted into SD-Trp media using Aria sorter (Beckman coulter). Recovered cells were induced again in second panning, 5×10⁶ cells were stained with 50 ng/ml P32-Biotin. Other reagents were at same concentration as in first panning. Top 3% of double positive yeast were collected. In the third panning cycle, 5×10⁶ cells were stained with 25 ng/ml p32-Biotin. The other reagents were at same concentration as in first panning. Top 3% of double positive yeast were collected. Sorted cells were plated on SD-Trp plates and 30 single colonies were picked and cultured.

Apparent K_{D} Calculations with Yeast

Native and mutated anti-p32 scFv-expressing yeast cells were stained for FACS analysis. First, the yeast cells were washed with PBS+0.5% ovalbumin. Next, the antigens were added in serial dilutions ranging from 1000-3.9 ng/μl in PBS+0.5% ovalbumin and mouse anti-c-myc (1:50), and incubated for an hour at room temperature. Cells were washed and incubated with APC-Streptavidin and Alexa Fluor 488 goat anti-mouse IgG1 (diluted 1:50 and 1:200, respectively). Finally, the cells were washed and resuspended in ice cold PBS and analyzed by FACS. The scFv expressing cells were gated and the geometric mean of antigen binding was calculated. Geometric mean was plotted versus antigen concentration and apparent K_{D} was calculated according to non-linear fit with one-site specific binding using GraphPad Prism 8.0.

Cloning of scFvp32 Colonies #8 and #20 in MSGV Retroviral Vector

Genes encoding for the scFv-p32 #8 and #20 were cloned into a pMSGV retroviral vector containing CD28 or 41BB costimulatory domain followed by FcγR (Globerson-Levin, A., Waks, T. & Eshhar, Z. 2014. “Elimination of progressive mammary cancer by repeated administrations of chimeric antigen receptor modified T cells”. Mol. Ther. 22, 1029-1038) or CD3zeta signaling domain. A T2A-mcherry fluorescent reporter cassette was added for easy assessment of transduction efficiency and a FLAG tag was added at the scFv N-terminus for evaluation of surface expression by FACS.

Sequences encoding scFv-32 #8 and scFv-32 #20 were amplified from the pETCON2 plasmid via PCR using primers for Gibson assembly cloning.

YST col FLAG VH for:
(SEQ ID NO: 4)
ACGATGACGACAAGGCTAGCGAGGTGCAGCTGGTGGAG;
YST col REV:
(SEQ ID NO: 5)
TGGTGATGATGATGGGGGGGGCCCCCTAGGACGGTCAG.

scFv P32 was first amplified by mixing 2 μl scFv template, 1 μl of each primer, 25 μl of Phusion hot start flex 2× master mix (NEB) and 21 μl water. PCR settings were 98 °C for 30 seconds followed by 30 cycles of 98 °C for 10 seconds, 60 °C for 30 seconds, 72 °C for 3 minutes and final incubation of 72 °C for 10 minutes. MSGV is a splicing-enhanced MSCV (murine stem cell virus) based retroviral vector, which was used to prepare retroviral particles in order to transduce T cells and obtain CAR T cells.
MSVG expressing the intracellular domain of CAR (41BBz or CD28g) construct was also amplified via PCR using primers for Gibson:

MSVG scP32His FOR:
(SEQ ID NO: 6)
AGCTGACCGTCCTAGGGGGCCCGCCCCATCAT;
MSVG VHscp32 Flag R:
(SEQ ID NO: 7)
GACTCCACCAGCTGCACCTCGCTAGCCTTGTC;

The backbone was amplified by mixing 2 μl of template, 1 μl of each primer, 25 μl of Phusion hot start flex 2× master mix (NEB) and 21 μl water. PCR settings were 98 °C for 30 seconds followed by 30 cycles of 98 °C for 10 seconds, 60 °C for 30 seconds, 72 °C for 3 minutes and final incubation of 72 °C for 10 minutes. PCR products were extracted from gel using Zymo-clean gel DNA recovery kit (D4001). scFv P32 inserts and the MSVG backbone were mixed in 3:1 ratio and 2× Gibson assembly master mix (NEB) and incubated for 1 hour at 50 °C. Followed by transformation, plasmids from possible clones were purified via mini prep and transfected into HEK293 cells to check for mcherry and Flag expression.
Generation of p32-Specific CAR and Retrovirus Production

For production of stable cell line which will produce new MSVG-CAR scp32 #8 (col8)/col 20 based P32 the present inventors used the GAL-V RD114 system. 293 Vec GAL-V and 293 vec RD114 cells were provided by BioVecPharma. 293 Vec GAL-V cells were transfected with the MSGV-CAR construct vector and the supernatant containing the retroviral particles were collected at 48 hours. Viral media was filtered and Polyberen was added to a final concentration of 8 μg/ml. Infection of 293 Vec RD114 cells was done by using this viral supernatant media and centrifuging the cells with this media for 10 minutes with no breaks at room temperature, and incubated overnight. Following infection, 293 Vec RD114 cells were sorted to select only the CAR expressing packaging cells.

### Transduction of Primary Lymphocytes

To generate p32 hCAR T cells, peripheral blood mononuclear cells (PBMCs) were separated on Lymphoprep (Ferenius Kabi Norge AS) centrifuge gradient, and activated on non-tissue culture 6-well plates which were pre-coated with antiCD3 and antiCD28 (Biolegend) antibodies for 48 hours. On day 2, activated T cells (2.5×10⁶ cells/well) were transduced with retroviral supernatant obtained from either CAR-PG13 or CAR-RD114 cells using “spin-infection” on RetroNectin 6-well coated plates with addition of 100 U/ml IL-2 (PeproTech).

In a similar fashion, to obtain p32 mCAR T cells, retroviral supernatant obtained from CAR-GPE86 packaging cell line was used to transduce activated murine splenocytes by “spin-infection” on RetroNectin plates (5×10⁶ cells/well). Splenocytes from syngeneic mice were activated with soluble antiCD3 (clone 145-2C11, Biolegend) and antiCD28 (clone 37.51, Biolegend) and 100 U/mL of IL-2 for 2 days before transduction was performed.

Transduction efficiency was estimated by analyzing FLAG cell surface expression, as well as mcherry expression on retrovirally transduced cells and comparing to either isotype control stained or un-transduced T cells (UT).

### CAR T-Cell Tumor Recognition

Cytotoxicity—For the specific lysis assays, luciferase-expressing tumor targets (1×10⁴ cells) were cocultured with varying amounts of p32 CAR T, or UT T cells, for 18 hour. Luminescence was measured using a IVIS lumina III™ system (Perkin Elmer) immediately upon addition of Luciferin (D-Luciferin Firefly, potassium salt Cat No. LUCK250, GOLDBIO, Gold Biotechnology). Other cytotoxicity assays included co-culture of GFP positive target cells with varying amounts of p32 CAR T, or UT T cells for 3-5 days. Cells were collected, stained and analyzed by flow cytometry.

### Proliferation Assay

For proliferation assay, T cells were labeled with CellTrace™ Violet probe (Molecular Probes™ Thermo Fisher Scientific), according to manufacturer's instruction and co-cultured with glioma cells at an ET ratio of either 3:1 or 1:1. CellTrace™ MViolet dilution was analyzed gating on CAR T+ (using anti-CD3 antibody and mcherry/T cells (anti-CD3+ and mcherry negative for un-transduced control cells) on day 3 using flow cytometry.

### Flow Cytometry and Immunofluorescence Staining

Efficiency of T cell transduction was assessed using anti-FLAG antibody (14793, Cell Signaling) along with mcherry fluorescent reporter. For flow cytometry analysis 100,000 cells were stained with the appropriated antibodies according to the antibodies manufacture's instructions. Briefly, the cells were incubated with TruStain FcX™-Fc blocker CD16/32 (Biolegend). Samples were then stained with different combinations of antibodies (anti-p32 PE (60.11) Santa Cruz sc-23884; anti-mouseCD68, Invitrogen, Cat #14-0681-82; Anti-mouse CD11b-BV785 BioLegend, Cat #101243; Anti-mouse CD45-FITC, BioLegend, Cat #147709). Cells were washed and resuspended in phosphate buffered saline (PBS) prior to acquisition on Attune NxT Flow Cytometer and analysis was performed using Kaluza software.

For confocal fluorescence imaging analysis, mice bearing glioma tumors (005) were treated with either Sp6 (irrelevant control) or p32-specific CAR T cells and perfused with 1×PBS and fixed with 4% paraformaldehyde. Brains were collected and coronal sections (30-40 μm) were cut using a HM450 Microtome (ThermoFisher Scientific) and images were obtained using a Zeiss LSM 800 Confocal Microscope.

### Tissue Processing

Brain tumors (GFP+ tissue resected under fluorescent microscope) were dissociated using a Neural dissociation kit (Miltenyi Biotec) according to manufacturer's instructions and the resulting cell suspension was cleaned of debris (myelin) via Percoll (Sigma) density gradient centrifugation. CD45-cells were enriched using anti-mouse-CD45 magnetic-microbead and MS columns (Miltenyi Biotec) according to manufacturer's instructions.

### Example 1

### Random Mutagenesis Revealed Improved P32 Scfv Antibodies

### Experimental Results

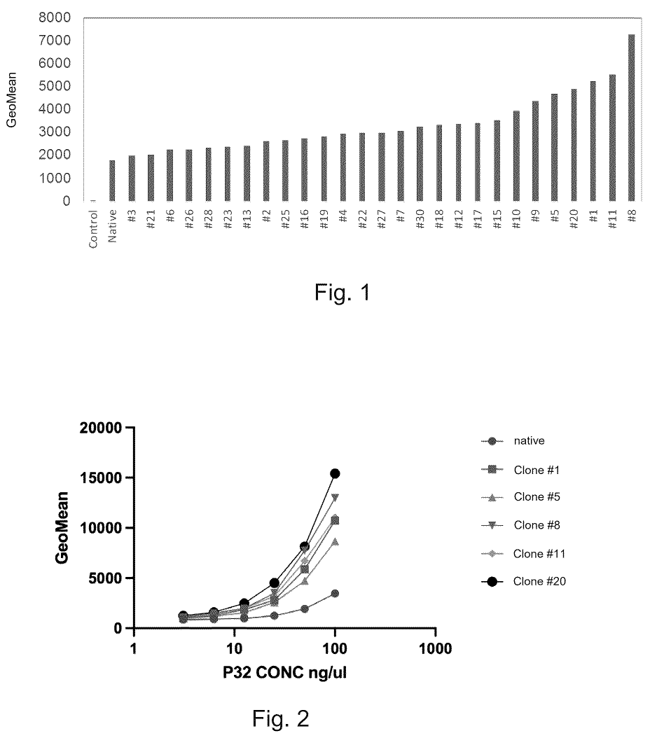

The present inventors have performed random site-direct mutagenesis on the “native” anti-p32 scFv antibody [which comprises SEQ ID NO: 67 (the V_{H} coding sequence) and SEQ ID NO: 69 (the V_{L} coding sequence), and generated a yeast display library in order to improve the affinity and binding of the scFv p32 antibody. Following three rounds of FACS sorting and enrichment (panning), the top 3-5% of binders to p32 were collected. Out of 30 single clones isolated, 27 of them showed improved binding (measured by flow cytometry-Geo mean fluorescence value) as compared to native scFv p32 (FIG. 1). From the top 10 clones, 5 were selected to measure approximate Kd values compared to native scFv p32 and the results confirmed improved recognition of all mutant clones tested (FIG. 2).

### Example 2

### The Chimeric Antigen Receptor (CAR) T Cells with the Improved P32 SCFV Exhibit Significant Cytotoxic Effects on Cancer Cells

### Experimental Results

Engineered p32 CAR T cells with the improved p32 scFv antibodies decrease proliferation of glioma cells and reduces percentage of residual tumor cells—The present inventors have generated two CAR constructs which included the p32 scFV from two representative clones (#8 and #20), as well as the 41BB and CD3z sequences (representative schemes are shown in FIGS. 3A-C) and compared their effect on proliferation in vitro on U251 glioma cells to that of CAR T cells expressing the native p32 scFv antibody. FIG. 3D shows that the CAR T cells expressing clones #20 or #8 p32 scFv significantly reduce the proliferation of U251 glioma cells by about 10-20 folds as compared to CAR T cells expressing the native p32 scFv. FIG. 3E shows that the CAR T cells expressing clones #20 or #8 p32 scFv significantly reduce the percentage of residual tumor cells compared to CAR T cells expressing the native p32 scFv. U251 glioma cells expressing GFP (target cells) were co-culture with varying amounts of p32 CAR T, or UT T cells for 3-5 days. Cells were collected, stained anti-CD3 and analyzed by flow cytometry. Taken together, these results show that CAR T cells expressing clones #20 or #8 p32 scFv have an improved function in terms of proliferation and killing capability when compared to native p32 scFv CAR T-cells.

Table 3, herein below, provides the FR1-4 and CDR1-3 sequences of the heavy chain and light chains of the native p32 compared to the p32-Col8 and p32-Col20 mutated antibodies generated and analyzed by present inventors.

TABLE 3

| Ab | FR1 | CDR1 | FR2 | CDR2 | FR3 | CDR3 | FR4 |
|---|---|---|---|---|---|---|---|
| Native- | EVQLVESGGG | SYGM | WVRQ | VISYDG | RFTISRDNSKNT | NLR | WGQ |
| heavy | LVQPGGSLRLS | H | APGKG | SNKYYA | LYLQMNSLRAE | HEK | GTLV |
| | CAASGFTFS | | LEWVA | DSVKG | DTAVYYCAR | Q | TVSS |
| SEQ ID | 8 | 9 | 10 | 11 | 12 | 13 | 14 |
| NO: | | | | | | | |
| Native- | QSVLTQPPSAS | SGSSS | WYQQL | RNNQRP | GVPDRFSGSKSG | AAW | FGGG |
| light | GTPGQRVTISC | NIGS | PGTAP | S | TSASLAISGLRSE | DDS | TKLT |
| | | NYVY | KLLIY | | DEADYYC | LFV | VL |
| SEQ ID | 15 | 16 | 17 | 18 | 19 | 20 | 21 |
| NO: | | | | | | | |
| Col 8- | EVQLVESGGG | SYGM | WVRQ | VISYDG | RFTISRDNSKNT | NLR | WGQ |
| Heavy* | LVQPGGSLRLS | H | APGKG | SNKYYA | LYLQMNSLRAE | HEK | GTLV |
| | CAASGFTFS | | LEWVA | DSVKG | DTAVYYCAR | Q | TVSS |
| SEQ ID | 22 | 23 | 24 | 25 | 26 | 27 | 28 |
| NO: | | | | | | | |
| Col 8- | QSVLTQPPSAS | SGSSS | WYRQL | RNNHRP | GVPDRFSGSKSG | AAW | FGGG |
| Light | GTPGQRVTSSC | NIGS | PGTAP | S | TSASLAISGLRSE | DDR | TKLT |
| | | NYVY | KLLIY | | DEADYYC | LFV | VL |
| SEQ ID | 29 | 30 | 31 | 32 | 33 | 34 | 35 |
| NO: | | | | | | | |
| Col 20 - | EVQLVESGGG | SYGM | WVRQ | VISYDG | RFTISRDNSKNT | NLR | WGQ |
| Heavy | LVQPGGSLRLS | H | APGKG | SNKYYA | LYLRMNSLRAE | HEK | GTLV |
| | CAASGFTFS | | LERVA | DSVKG | DTAVYYCAR | Q | TVSS |
| SEQ ID | 36 | 37 | 38 | 39 | 40 | 41 | 42 |
| NO: | | | | | | | |
| Col 20- | QSVLTQPPSAS | SGSSS | WYRH | RKNQRP | GVPDRFSGSKSG | AAW | FGGG |
| Light | GTPGQRVTISC | NIGS | LPGTA | S | TSASLAISGLRSE | DDS | TKLT |
| | | NYVY | PKLLIY | | DEADYYC | LFV | VL |
| SEQ ID | 43 | 44 | 45 | 46 | 47 | 48 | 49 |
| NO: | | | | | | | |

Table 3. Bolded and underlined amino acids are mutated compared to the native p32 antibody sequences.
* It is noted that the p32 Col 8 heavy chain sequence (SEQ ID NO: 92) is identical to the p32 native heavy chain sequence (SEQ ID NO: 53).

Table 4, herein below, provides the FR1-4 and CDR1-3 and scFv of the heavy chain and light chains of the mutated antibodies generated and analyzed by present inventors.

TABLE 4

| | | SEQ ID |
|---|---|---|
| | Amino Acid Sequence | NO: |
| Clone | | |
| #1 | Variable Heavy Chain | |
| FR1 | EVQLVESGGGLVQPGGSLRLSCAASGFTFS | 116 |
| CDR1 | SYGMH | 117 |
| FR2 | WVRQAPGKGLEWVA | 118 |
| CDR2 | VISYDGSNKYYADSVKG | 119 |
| FR3 | RFTISRDNSKNTLYMQMNSLRAEDTAVYYCAR | 120 |
| CDR3 | NLRHEKQ | 121 |
| FR4 | WGRGTLVTVSS | 122 |
| Clone | | |
| #1 | Variable Light Chain | |
| FR1 | QSVLTQTPSASGTPGQRVTISR | 123 |
| CDR1 | SGSSSNIGSNYVY | 124 |
| FR2 | WYQQLPGTAPKLLIY | 125 |
| CDR2 | RNNQRPS | 126 |
| FR3 | GVHDRFSGSKSGTSASLAISGLRSEDEADYYC | 127 |
| CDR3 | AAWDDSLFV | 128 |
| FR4 | FGGGTKLTVL | 129 |
| p32 | EVQLVESGGGLVQPGGSLRLSCAASGFTFSSYGMHWVRQAPGKGL | 100 |
| scFv | EWVAVISYDGSNKYYADSVKGRFTISRDNSKNTLYMQMNSLRAED | |
| Clone | TAVYYCARNLRHEKQWGRGTLVTVSSGGGGSGGGGSGGSALQSVL | |
| #1 | TQTPSASGTPGQRVTISRSGSSSNIGSNYVYWYQQLPGTAPKLLI | |
| | YRNNQRPSGVHDRFSGSKSGTSASLAISGLRSEDEADYYCAAWDD | |
| | SLFVFGGGTKLTVL | |
| Clone | | |
| #5 | Variable Heavy Chain | |
| FR1 | EVQLVESGGGLVQPGGSLRLSCAASGFTFS | 131 |
| CDR1 | SYGMH | 132 |
| FR2 | WVRQAPGKGLEWVA | 133 |
| CDR2 | VISYDGSNKYYADSVKG | 134 |
| FR3 | RFTISRDNSKNTLYLQMNSLRAEDTAVYYCAR | 135 |
| CDR3 | NLRHEKQ | 136 |
| FR4 | WGQGTLVTVSS | 137 |
| Clone | | |
| #5 | Variable Light Chain | |
| FR1 | QSVLTQPPSASGTPGQRVTISY | 138 |
| CDR1 | SGSSSNIGSNYVY | 139 |
| FR2 | WYQQLPGTAPKLLIY | 140 |
| CDR2 | RNNQRPS | 141 |
| FR3 | GVPDRFSGSKSGTSASLAISGLRSEDEADYYC | 142 |
| CDR3 | AAWDDSLFV | 143 |
| FR4 | FGGGTKLTVL | 144 |
| p32 | EVQLVESGGGLVQPGGSLRLSCAASGFTFSSYGMHWVRQAPGKGL | 101 |
| scFv | EWVAVISYDGSNKYYADSVKGRFTISRDNSKNTLYLQMNSLRAED | |
| Clone | TAVYYCARNLRHEKQWGQGTLVTVSSGGGGSGGGGSGGSALQSVL | |
| #5 | TQPPSASGTPGQRVTISYSGSSSNIGSNYVYWYQQLPGTAPKLLI | |
| | YRNNQRPSGVPDRFSGSKSGTSASLAISGLRSEDEADYYCAAWDD | |
| | SLFVFGGGTKLTVL | |
| Clone | | |
| #8 | Variable Heavy Chain | |
| FR1 | EVQLVESGGGLVQPGGSLRLSCAASGFTFS | 22 |
| CDR1 | SYGMH | 23 |
| FR2 | WVRQAPGKGLEWVA | 24 |
| CDR2 | VISYDGSNKYYADSVKG | 25 |
| FR3 | RFTISRDNSKNTLYLQMNSLRAEDTAVYYCAR | 26 |
| CDR3 | NLRHEKQ | 27 |
| FR4 | WGQGTLVTVSS | 28 |
| Clone | | |
| #8 | Variable Light Chain | |
| FR1 | QSVLTQPPSASGTPGQRVTSSC | 29 |
| CDR1 | SGSSSNIGSNYVY | 30 |
| FR2 | WYRQLPGTAPKLLIY | 31 |
| CDR2 | RNNHRPS | 32 |
| FR3 | GVPDRFSGSKSGTSASLAISGLRSEDEADYYC | 33 |
| CDR3 | AAWDDRLFV | 34 |
| FR4 | FGGGTKLTVL | 35 |
| p32 | EVQLVESGGGLVQPGGSLRLSCAASGFTFSSYGMHWVRQAPGKGL | 110 |
| scFv | EWVAVISYDGSNKYYADSVKGRFTISRDNSKNTLYLQMNSLRAED | |
| Clone | TAVYYCARNLRHEKQWGQGTLVTVSSGGGGSGGGGSGGSALQSVL | |
| #8 | TQPPSASGTPGQRVTSSCSGSSSNIGSNYVYWYRQLPGTAPKLLI | |
| | YRNNHRPSGVPDRFSGSKSGTSASLAISGLRSEDEADYYCAAWDD | |
| | RLFVFGGGTKLTVL | |
| Clone | | |
| #9 | Variable Heavy Chain | |
| FR1 | EVQLVESGGGLVQPGGSLRLSCAASGFTFS | 146 |
| CDR1 | SYCMH | 147 |
| FR2 | WVRQAPGKGLEWVA | 148 |
| CDR2 | VISYDGSNKYCADSVKG | 149 |
| FR3 | RFTISRDNSKNTLYLQMNSLRAEDTAVYYCAR | 150 |
| CDR3 | NLRHEKQ | 151 |
| FR4 | WGQGTLVTVSS | 152 |
| Clone | | |
| #9 | Variable Light Chain | |
| FR1 | QSVLTQPPSASGTPGQRVTISC | 153 |
| CDR1 | SGSSSNIGSNYVY | 154 |
| FR2 | WYQQLPGTAPKLLIY | 155 |
| CDR2 | RNDQRPS | 156 |
| FR3 | GVPDRFSGSKSGTSASPAISGLRFEDEADYYS | 157 |
| CDR3 | AARDDSLFV | 158 |
| FR4 | FGGGTKLTVL | 159 |
| p32 | EVQLVESGGGLVQPGGSLRLSCAASGFTFSSYCMHWVRQAPGKGL | 102 |
| scFv | EWVAVISYDGSNKYCADSVKGRFTISRDNSKNTLYLQMNSLRAEDT | |
| Clone | AVYYCARNLRHEKQWGQGTLVTVSSGGGGSGGGGSGGSALQSVLTQ | |
| #9 | PPSASGTPGQRVTISCSGSSSNIGSNYVYWYQQLPGTAPKLLIYRN | |
| | DQRPSGVPDRFSGSKSGTSASPAISGLRFEDEADYYSAARDDSLFV | |
| | FGGGTKLTVL | |
| Clone | | |
| #10 | Variable Heavy Chain | |
| FR1 | EVQLVESGGGLVQPGGSLRLSCAASGFTFS | 130 |
| CDR1 | SYGMH | 145 |
| FR2 | WVRQAPGKGLEWVA | 160 |
| CDR2 | VISYDGSNKYYADSVKG | 161 |
| FR3 | RFTISRDNSKNTLYLQMNSLRAEDTAVYYCAR | 162 |
| CDR3 | NLRHEKQ | 163 |
| FR4 | WGQGTLVTVSS | 164 |
| Clone | | |
| #10 | Variable Light Chain | |
| FR1 | QSVLTQPPSASGTPGQRVTISC | 165 |
| CDR1 | SGSSSNIGSNYVY | 166 |
| FR2 | WYQQLPGTAPKLLIY | 167 |
| CDR2 | RNNQRPS | 168 |
| FR3 | GVPDRFSGSKSGTSASLAISGLRSEDEADYYR | 169 |
| CDR3 | AAWDDSLFV | 170 |
| FR4 | FGGGTKLTVL | 171 |
| p32 | EVQLVESGGGLVQPGGSLRLSCAASGFTFSSYGMHWVRQAPGKGL | 103 |
| scFv | EWVAVISYDGSNKYYADSVKGRFTISRDNSKNTLYLQMNSLRAED | |
| Clone | TAVYYCARNLRHEKQWGQGTLVTVSSGGGGSGGGGGSGGSALQSV | |
| #10 | LTQPPSASGTPGQRVTISCSGSSSNIGSNYVYWYQQLPGTAPKLL | |
| | IYRNNQRPSGVPDRFSGSKSGTSASLAISGLRSEDEADYYRAAWD | |
| | DSLFVFGGGTKLTVL | |
| Clone | | |
| #11 | Variable Heavy Chain | |
| FR1 | EVQLVESGGGLVQPGGSLRLSCAASGFTFS | 172 |
| CDR1 | SYGMH | 173 |
| FR2 | WVRQAPGKGLEWVA | 174 |
| CDR2 | VISYDGSNKYYADSVKG | 175 |
| FR3 | RFTISRDNSKNTLYLQMNSLRAEDTAVYYCAR | 176 |
| CDR3 | NLRHEKQ | 177 |
| FR4 | WGQGTLVTVSS | 178 |
| Clone | | |
| #11 | Variable Light Chain | |
| FR1 | QSVLTQPPSASGTPGQRVTISC | 179 |
| CDR1 | SGSSSNIGSNYVY | 180 |
| FR2 | WYQQLPGTAPKLLIY | 181 |
| CDR2 | RNNQRPS | 182 |
| FR3 | GVPDRFSGSKSGTTASLAISGLRSEDEADYYS | 183 |
| CDR3 | AAWDDSLFV | 184 |
| FR4 | FGGGTKLTVL | 185 |
| p32 | EVQLVESGGGLVQPGGSLRLSCAASGFTFSSYGMHWVRQAPGKGL | 104 |
| scFv | EWVAVISYDGSNKYYADSVKGRFTISRDNSKNTLYLQMNSLRAED | |
| Clone | TAVYYCARNLRHEKQWGQGTLVTVSSGGGGSGGGGSGGSALQSVL | |
| #11 | TQPPSASGTPGQRVTISCSGSSSNIGSNYVYWYQQLPGTAPKLLI | |
| | YRNNQRPSGVPDRFSGSKSGTTASLAISGLRSEDEADYYSAAWDD | |
| | SLFVFGGGTKLTVL | |
| Clone | | |
| #12 | Variable Heavy Chain | |
| FR1 | EVQLVESGGGLVQPRGSLRLSCAASGFTFS | 186 |
| CDR1 | SEGMH | 187 |
| FR2 | WVRQAPGKGLEWVA | 188 |
| CDR2 | VISYDGSNKYYADSVKG | 189 |
| FR3 | RFTISRDNSKNTLYLQMNSLRAEDTAVYYCAR | 190 |
| CDR3 | NLRHEKQ | 191 |
| FR4 | WGQGTLVTVSS | 192 |
| Clone | | |
| #12 | Variable Light Chain | |
| FR1 | QSVLTQPPSASGTPRQRVTISS | 193 |
| CDR1 | SGSSSNIGSNYVY | 194 |
| FR2 | WYQQLPGTAPKLLIY | 195 |
| CDR2 | RNNQRPS | 196 |
| FR3 | GAPDRFSGSKSGTSASRAISGLRSEDEADYYR | 197 |
| CDR3 | AAWDDSLFV | 198 |
| FR4 | FGGGTKLTVL | 199 |
| p32 | EVQLVESGGGLVQPRGSLRLSCAASGFTFSSEGMHWVRQAPGKGLE | 105 |
| scFv | WVAVISYDGSNKYYADSVKGRFTISRDNSKNTLYLQMNSLRAEDT | |
| Clone | AVYYCARNLRHEKQWGQGTLVTVSSGGGGSGGGGSGGSALQSVLTQ | |
| #12 | PPSASGTPRQRVTISSSGSSSNIGSNYVYWYQQLPGTAPKLLIYRN | |
| | NQRPSGAPDRFSGSKSGTSASRAISGLRSEDEADYYRAAWDDSLFV | |
| | FGGGTKLTVL | |
| Clone | | |
| #15 | Variable Heavy Chain | |
| FR1 | EAQLVESGGGLVQPGGSLRLSCAASGFTFS | 200 |
| CDR1 | SYGMH | 201 |
| FR2 | WVRQAPGKGLEWVA | 202 |
| CDR2 | VISYDGSNKYYADSVKG | 203 |
| FR3 | RFTISRDNSKNTLYLQMNSLRAEDTAVYYCAR | 204 |
| CDR3 | NLRHEKQ | 205 |
| FR4 | WGQGTLVTVSS | 206 |
| Clone | | |
| #15 | Variable Light Chain | |
| FR1 | QSVLTQPPSASGTPGQRVTISC | 207 |
| CDR1 | SGSSSNIGSNYVY | 208 |
| FR2 | WYQQLPGTAPKLLIY | 209 |
| CDR2 | RNNQRPS | 210 |
| FR3 | GVPDRFSGSKSGTSASLAISGLRSEDEADYYR | 211 |
| CDR3 | AAWDDSLFV | 212 |
| FR4 | FGGGTKLTVL | 213 |
| p32 | EAQLVESGGGLVQPGGSLRLSCAASGFTFSSYGMHWVRQAPGK | 107 |
| scFv | GLEWVAVISYDGSNKYYADSVKGRFTISRDNSKNTLYLQMNSLRA | |
| Clone | EDTAVYYCARNLRHEKQWGQGTLVTVSSGGGGSGGGGSGGSALQSV | |
| #15 | LTQPPSASGTPGQRVTISCSGSSSNIGSNYVYWYQQLPGTAPKLLI | |
| | YRNNQRPSGVPDRFSGSKSGTSASLAISGLRSEDEADYYRAAWDDS | |
| | LFVFGGGTKLTVL | |
| Clone | | |
| #17 | Variable Heavy Chain | |
| FR1 | EVQLVESGGGLVQPGGSLRLSCAASGFTFS | 214 |
| CDR1 | SYGMH | 215 |
| FR2 | WVRQAPGKGLERVA | 216 |
| CDR2 | VISYDGSNKYYADSAKG | 217 |
| FR3 | RFAVSRDNSKNTLYLRMNCLRAEDTAVYYCAR | 218 |
| CDR3 | NLRHEKQ | 219 |
| FR4 | WGQGTLVTVSR | 220 |
| Clone | | |
| #17 | Variable Light Chain | |
| FR1 | QSVLTQPPSASGTPGQRVIISC | 221 |
| CDR1 | SGSSSNIGSNYVY | 222 |
| FR2 | WYQQLPGTAPKLLIY | 223 |
| CDR2 | RNNQRPS | 224 |
| FR3 | GVPDRFSGSKSGTSASLAISGLRSEDEADYYC | 225 |
| CDR3 | AAWDDSLFV | 226 |
| FR4 | FGGGTKLTVL | 227 |
| p32 | EVQLVESGGGLVQPGGSLRLSCAASGFTFSSYGMHWVRQAPGKGL | 108 |
| scFv | ERVAVISYDGSNKYYADSAKGRFAVSRDNSKNTLYLRMNCLRAED | |
| Clone | TAVYYCARNLRHEKQWGQGTLVTVSRGGGGSGGGGSGGSALQSVL | |
| #17 | TQPPSASGTPGQRVIISCSGSSSNIGSNYVYWYQQLPGTAPKLLI | |
| | YRNNQRPSGVPDRFSGSKSGTSASLAISGLRSEDEADYYCAAWDD | |
| | SLFVFGGGTKLTVL | |
| Clone | | |
| #20 | Variable Heavy Chain | |
| FR1 | EVQLVESGGGLVQPGGSLRLSCAASGFTFS | 36 |
| CDR1 | SYGMH | 37 |
| FR2 | WVRQAPGKGLERVA | 38 |
| CDR2 | VISYDGSNKYYADSVKG | 39 |
| FR3 | RFTISRDNSKNTLYLRMNSLRAEDTAVYYCAR | 40 |
| CDR3 | NLRHEKQ | 41 |
| FR4 | WGQGTLVTVSS | 42 |
| Clone | | |
| #20 | Variable Light Chain | |
| FR1 | QSVLTQPPSASGTPGQRVTISC | 43 |
| CDR1 | SGSSSNIGSNYVY | 44 |
| FR2 | WYRHLPGTAPKLLIY | 45 |
| CDR2 | RKNQRPS | 46 |
| FR3 | GVPDRFSGSKSGTSASLAISGLRSEDEADYYC | 47 |
| CDR3 | AAWDDSLFV | 48 |
| FR4 | FGGGTKLTVL | 49 |
| p32 | EVQLVESGGGLVQPGGSLRLSCAASGFTFSSYGMHWVRQAPGKGL | 111 |
| scFv | ERVAVISYDGSNKYYADSVKGRFTISRDNSKNTLYLRMNSLRAEDT | |
| Clone | AVYYCARNLRHEKQWGQGTLVTVSSGGGGSGGGGSGGSALQSVLTQ | |
| #20 | PPSASGTPGQRVTISCSGSSSNIGSNYVYWYRHLPGTAPKLLIYRK | |
| | NQRPSGVPDRFSGSKSGTSASLAISGLRSEDEADYYCAAWDDSLFV | |
| | FGGGTKLTVL | |
| Clone # | | |
| 14 | Variable Heavy Chain | |
| FR1 | EVQPVESGGGLVQPGGSLRLSCAASGFTFS | 228 |
| CDR1 | SYGMH | 229 |
| FR2 | WVRQAPGKGLEWVA | 230 |
| CDR2 | VISYDGSNKYYADSVKG | 231 |
| FR3 | RYTISRDNSKNTLYLQMNSLRAEDTAVYYCAR | 232 |
| CDR3 | NLRHEKQ | 233 |
| FR4 | WGQGTLVTVSS | 234 |
| Clone # | | |
| 14 | Variable Light Chain | |
| FR1 | QSVLTQPPSASGTPGQRVTISC | 235 |
| CDR1 | SGSSSNIGSNYVY | 236 |
| FR2 | WYQQLPGTAPKLLIY | 237 |
| CDR2 | RNNQRPS | 238 |
| FR3 | GVPDRFSGSKSGTSASLAISGLRSEDEADYYC | 239 |
| CDR3 | AAWDDSLFV | 240 |
| FR4 | FGGGTKLTVL | 241 |
| p32 | EVQPVESGGGLVQPGGSLRLSCAASGFTFSSYGMHWVRQAPGKGL | 106 |
| scFv | EWVAVISYDGSNKYYADSVKGRYTISRDNSKNTLYLQMNSLRAED | |
| Clone | TAVYYCARNLRHEKQWGQGTLVTVSSGGGGSGGGGSGGSALQSVL | |
| #14 | TQPPSASGTPGQRVTISCSGSSSNIGS | |
| | NYVYWYQQLPGTAPKLLIYRNNQRPSGVPDRFSGSKSGTSASLAI | |
| | SGLRSEDEADYYCAAWDDSLFVFGGGTKLTVL | |
| Clone | | |
| #24 | Variable Heavy Chain | |
| FR1 | EVQLVESGGGLVQPGGSLRLSCAASGFTFS | 242 |
| CDR1 | SYGMH | 243 |
| FR2 | WVRQAPGKGLEWVA | 244 |
| CDR2 | VISYDGSNKYYADSVKG | 245 |
| FR3 | RFTISRDNSKNTLYLQMNSLRAEDTAVYCCAR | 246 |
| CDR3 | NLRHEKQ | 247 |
| FR4 | WGQGTLVTVSS | 248 |
| Light | | |
| FR1 | QSVLTQPPSASGTPGQRVTISC | 249 |
| CDR1 | SGSSSNIGSNYVY | 250 |
| FR2 | WYQQHPGTAPKLLIY | 251 |
| CDR2 | RNNQRPS | 252 |
| FR3 | GVPDRFSGSKSGTSASLAISGLRSEDEADYYC | 253 |
| CDR3 | AAWDDSLFV | 254 |
| FR4 | FGGGTKLTVL | 255 |
| p32 | EVQLVESGGGLVQPGGSLRLSCAASGFTFSSYGMHWVRQAPGKGL | 109 |
| scFv | EWVAVISYDGSNKYYADSVKGRFTISRDNSKNTLYLQMNSLRAEDT | |
| Clone | AVYCCARNLRHEKQWGQGTLVTVSSGGGGSGGGGSGGSALQSVLT | |
| #24 | QPPSASGTPGQRVTISCSGSSSNIGSN | |
| | YVYWYQQHPGTAPKLLIYRYNQRPSGVPDRFSGSKSGTSASLAISGL | |
| | RSEDEADYYCAAWDDSLFVFGGGTKLTVL | |

Table 4. Underlined amino acids are mutated compared to the native p32 antibody sequences.

Table 5 provides the variable heavy and light chains of selected antibodies which are described herein.

TABLE 5

| Description | Amino acid SEQ ID NO: | Nucleic acid SEQ ID NO: |
|---|---|---|
| Native p32 V_{H} | 53 | 67 |
| Native p32 V_{L} | 55 | 69 |
| P32 Col#8 V_{H}* | 92 | 94 |
| P32 Col#8 V_{L} | 93 | 95 |
| P32 Col#20 V_{H} | 96 | 98 |
| P32 Col#8 V_{L} | 97 | 99 |

*The V_{H} sequence of p32 Col#8 is identical to the V_{H} sequence of p32 native.

Table 6 describes the sequence identifiers of the VH and VL sequences of the isolated antibodies.

TABLE 6

| | V_{H} amino acid sequence | V_{L} amino acid sequence |
|---|---|---|
| P32 Antibody name | SEQ ID NO: | SEQ ID NO: |
| native | 53 | 55 |
| Clone # 8 | 92 | 93 |
| Clone # 20 | 96 | 97 |
| Clone #1 | 256 | 257 |
| Clone #5 | 258 | 259 |
| Clone #9 | 260 | 261 |
| Clone #10 | 262 | 263 |
| Clone #11 | 264 | 265 |
| Clone #12 | 266 | 267 |
| Clone #14 | 268 | 269 |
| Clone #15 | 270 | 271 |
| Clone #17 | 272 | 273 |
| Clone #24 | 274 | 275 |

### Example 3

### Superior Killing Activity of the Native SCP32-41BB-Zeta Car Over the Native SCP32-CD28-Gamma Car

### Experimental Results

The present inventors have performed a side by side comparison of the native anti-p32 scFv fused to the intracytoplasmic domains CD28-FcRgamma (FIG. 4A) with the same native anti-p32 scFv extracellular domain but when fused to 41BB-CD3zeta domains (FIG. 4B). Transduction of human T cells showed the same level of expression on the surface of both CAR constructs and similar phenotype in terms of CD4/CD8 ratios (FIGS. 4B-C). All stimulated cells showed expression of CD25 on surface (FIG. 4E) with p32-41BB-CD3zeta showing a population with higher expression of this activation marker. Two different assays were performed to assess the cytotoxic effect of the CARs compared to un-transduced (UT) T cells (controls). Glioma patient derived cell line (GBM83) expressing the luciferase reporter were co-cultured with CAR transduced T cells or UT controls at different effector to target ratios. As shown in FIGS. 4F-G, the p32-41BB-CD3zeta CAR showed superior killing capacity compared to p32-CD28-FcRgamma. Similar results were obtained when the transduced CAR T cells and UT cells were co-cultured with GBM83 GFP+ cells and the cytotoxic activity was analyzed by flow cytometry analysis (FIGS. 4H-K). These results show a more significant killing effect for the T cells transduced with the p32-41BBz CAR construct as compared to the T cells transduced with the p32-CD28g CAR construct. In addition, both transduced T cells had a superior effect when compared to the un-transduced T cells. Altogether, these experiments support a superior killing activity of the native scFv p32-41BB-CD3zeta CAR when compared to native scFv p32-CD28-FcRgamma CAR construct.

### Example 4

### P32 Expression on Macrophages is Specific to the Tumor Microenvironment

### Experimental Results

p32 CAR T cells can target tumor associated macrophages—The present inventors have examined the tumor effect on p32 expression patterns in RAW 264.7 macrophage cell line. RAW 264.7 is a macrophage cell line that was established from a tumor in a male mouse (ATCC TIB-71 cells). Incubation of the RAW 264.7 with a conditioned medium of the glioblastoma 005 cells (Tomotoshi Marumoto et al., 2009. “Development of a novel mouse glioma model using lentiviral vectors”. Nat Med. 15(1):110-6) resulted in induction of p32 cell surface expression on the macrophages (FIG. 5A). In addition, the present inventors have analyzed the expression of p32 in tumor associated macrophages. To that end, tumors collected from 005-injected mice were dissociated and analyzed by flow cytometry gating on CD68 positive cells (macrophage lineage marker). The results showed high expression of p32 in CD68+ tumor associated macrophages (FIG. 5C). No expression was detected in CD68+ cells from dissociated brains of healthy (marked as “WT”) mice (FIG. 5B). FIG. 5D shows that CD68+ tumor associated macrophages (TAMs) express p32 compared to isotype control antibody (histogram presentation). Finally, immunofluorescence analysis of tumors of mice treated with the p32 CAR-T cells showed a significant decrease in CD68+ cells in the tumor area when compared to mice that underwent irrelevant (mSP6)-CAR-T cell therapy (FIGS. 5E-F). In order to study this phenomenon outside the tumor parenchyma and advocate for the safety or the p32 CAR T treatment, the present inventors showed that macrophages in the periphery (circulating bone marrow macrophages) do not show p32 cell surface expression [FIG. 5H (from 005-tumor-bearing mice), showing almost no p32+ cells, similarly to FIG. 5G (from healthy (WT)] and that the previous effect in p32 expression is only local, in the tumor microenvironment.

Altogether, the results demonstrate that p32 is selectively expressed on tumor-associated macrophages within the tumor microenvironment, as confirmed by flow cytometry and immunofluorescence. Importantly, no p32 expression was observed in CD68+ macrophages from healthy tissue or circulating bone marrow macrophages, suggesting that p32 expression is tumor-specific and localized to TAMs. Treatment with p32-CAR T cells significantly reduced the presence of TAMs in tumors, highlighting their potential to reprogram the immunosuppressive tumor microenvironment. These findings suggest that p32-positive TAMs are a novel, tumor-restricted target for p32-CAR T cells, providing a promising therapeutic approach with minimal off-target effects on healthy macrophages.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES (ADDITIONAL PUBLICATIONS ARE CITED IN TEXT)

Liat Rousso-Noori, Ignacio Mastandrea, Shauli Talmor, Tova Waks, Anat Globerson Levin, Maarja Haugas, Tambet Teesalu, Luis Alvarez-Vallina, Zelig Eshhar & Dinorah Friedmann-Morvinski. “P32-specific CAR T cells with dual antitumor and antiangiogenic therapeutic potential in gliomas”. Nature Communications 2021; 12(1):3615.
Agemy L, Kotamraju V R, Friedmann-Morvinski D, Sharma S, Sugahara K N, Ruoslahti E. “Proapoptotic peptide-mediated cancer therapy targeted to cell surface p32”. Mol Ther. 2013 December; 21(12):2195-204.
Globerson-Levin, A., Waks, T. & Eshhar, Z. Elimination of progressive mammary cancer by repeated administrations of chimeric antigen receptor modified T cells. Mol. Ther. 22, 1029-1038 (2014).
Marumoto, T. et al. Development of a novel mouse glioma model using lentiviral vectors. Nat. Med. 15, 110-116 (2009).
Mao, P. et al. Mesenchymal glioma stem cells are maintained by activated glycolytic metabolism involving aldehyde dehydrogenase 1A3. Proc. Natl Acad. Sci. USA 110, 8644-8649 (2013).
Labanieh L, Majzner R G, Klysz D, Sotillo E, Fisher C J, Vilches-Moure J G, Pacheco K Z B, Malipatlolla M, Xu P, Hui J H, Murty T, Theruvath J, Mehta N, Yamada-Hunter S A, Weber E W, Heitzeneder S, Parker K R, Satpathy A T, Chang H Y, Lin M Z, Cochran J R, Mackall C L. Enhanced safety and efficacy of protease-regulated CAR-T cell receptors. Cell May 12; 185(10):1745 (2022).
Yenugonda V, Nomura N, Kouznetsova V, Tsigelny I, Fogal V, Nurmemmedov E, Kesari S, Babic I. “A novel small molecule inhibitor of p32 mitochondrial protein overexpressed in glioma”. J. Transl Med. 2017 Oct. 18; 15(1):210.
Fogal V, Babic I, Chao Y, Pastorino S, Mukthavaram R, Jiang P, Cho Y J, Pingle S C, Crawford J R, Piccioni D E, Kesari S. “Mitochondrial p32 is upregulated in Myc expressing brain cancers and mediates glutamine addiction”. Oncotarget. 2015 Jan. 20; 6(2):1157-70.
Sánchez-Martín D, Cuesta A M, Fogal V, Ruoslahti E, Alvarez-Vallina L. “The multicompartmental p32/gC1qR as a new target for antibody-based tumor targeting strategies”. J Biol Chem. 2011 Feb. 18; 286(7):5197-203.
Fogal V, Richardson A D, Karmali P P, Scheffler I E, Smith J W, Ruoslahti E. “Mitochondrial p32 protein is a critical regulator of tumor metabolism via maintenance of oxidative phosphorylation”. Mol Cell Biol. 2010 March; 30(6):1303-18.
Fogal V, Zhang L, Krajewski S, Ruoslahti E. “Mitochondrial/cell-surface protein p32/gC1qR as a molecular target in tumor cells and tumor stroma”. Cancer Res. 2008 Sep. 1; 68(17):7210-8.

What is claimed is:

1. An isolated antibody or a fragment thereof which binds to a human p32 tumor associated antigen (TAA), the antibody comprising a heavy chain variable domain (VH) and a light chain variable domain (VL) each comprising three complementarity-determining regions (CDRs), wherein:
(i) the VH CDRs 1, 2 and 3 comprise amino acid sequences SEQ ID NOs: 23, 25 and 27, respectively, and the VL CDRs 1, 2 and 3 comprise amino acid sequences SEQ ID NOs: 30, 32 and 34, respectively;
(ii) the VH CDRs 1, 2 and 3 comprise amino acid sequences SEQ ID NOs: 37, 39 and 41, respectively, and the VL CDRs 1, 2 and 3 comprise amino acid sequences SEQ ID NOs: 44, 46 and 48, respectively;
(iii) the VH CDRs 1, 2 and 3 comprise amino acid sequences SEQ ID NOs: 117, 119 and 121, respectively, and the VL CDRs 1, 2 and 3 comprise amino acid sequences SEQ ID NOs: 124, 126 and 128, respectively;
(iv) the VH CDRs 1, 2 and 3 comprise amino acid sequences SEQ ID NOs: 132, 134 and 136, respectively, and the VL CDRs 1, 2 and 3 comprise amino acid sequences SEQ ID NOs: 139, 141, 143, respectively;
(v) the VH CDRs 1, 2 and 3 comprise amino acid sequences SEQ ID NOs: 147, 149, 151, respectively, and the VL CDRs 1, 2 and 3 comprise amino acid sequences SEQ ID NOs: 154, 156 and 158, respectively;
(vi) the VH CDRs 1, 2 and 3 comprise amino acid sequences SEQ ID NOs: 145, 161 and 163, respectively, and the VL CDRs 1, 2 and 3 comprise amino acid sequences SEQ ID NOs: 166, 168, 170, respectively;
(vii) the VH CDRs 1, 2 and 3 comprise amino acid sequences SEQ ID NOs: 173, 175 and 177 respectively, and the VL CDRs 1, 2 and 3 comprise amino acid sequences SEQ ID NOs: 180, 182 and 184, respectively;
(viii) the VH CDRs 1, 2 and 3 comprise amino acid sequences SEQ ID NOs: 187, 189 and 191, respectively, and the VL CDRs 1, 2 and 3 comprise amino acid sequences SEQ ID NOs: 194, 196 and 198, respectively;
(ix) the VH CDRs 1, 2 and 3 comprise amino acid sequences SEQ ID NOs: 201, 203 and 205, respectively, and the VL CDRs 1, 2 and 3 comprise amino acid sequences SEQ ID NOs: 208, 210 and 212, respectively;
(x) the VH CDRs 1, 2 and 3 comprise amino acid sequences SEQ ID NOs: 215, 217 and 219, respectively, and the VL CDRs 1, 2 and 3 comprise amino acid sequences SEQ ID NOs: 222, 224 and 226, respectively;
(xi) the VH CDRs 1, 2 and 3 comprise amino acid sequences SEQ ID NOs: 229, 231 and 233, respectively, and the VL CDRs 1, 2 and 3 comprise amino acid sequences SEQ ID NOs: 236, 238 and 240, respectively; or
(xii) the VH CDRs 1, 2 and 3 comprise amino acid sequences SEQ ID NOs: 243, 245 and 247, respectively, and the VL CDRs 1, 2 and 3 comprise amino acid sequences SEQ ID NOs: 250, 252 and 254, respectively.

2. The isolated antibody or the fragment thereof of claim 1, wherein said antibody of
(i) further comprises heavy chain variable domain framework regions (FRs) 1, 2, 3 and 4 comprising amino acids SEQ ID NOs: 22, 24, 26 and 28, respectively;
(ii) further comprises heavy chain variable domain FRs 1, 2, 3 and 4 comprising amino acids SEQ ID NOs: 36, 38, 40 and 42, respectively;
(iii) further comprises heavy chain variable domain FRs 1, 2, 3 and 4 comprising amino acids SEQ ID NOs: 116, 118, 120 and 122, respectively;
(iv) further comprises heavy chain variable domain FRs 1, 2, 3 and 4 comprising amino acids SEQ ID NOs: 131, 133, 135 and 137, respectively;
(v) further comprises heavy chain variable domain FRs 1, 2, 3 and 4 comprising amino acids SEQ ID NOs: 146, 148, 150 and 152, respectively;
(vi) further comprises heavy chain variable domain FRs 1, 2, 3 and 4 comprising amino acids SEQ ID NOs: 130, 160, 162 and 164, respectively;
(vii) further comprises heavy chain variable domain FRs 1, 2, 3 and 4 comprising amino acids SEQ ID NOs: 172, 174, 176 and 178, respectively;
(viii) further comprises heavy chain variable domain FRs 1, 2, 3 and 4 comprising amino acids SEQ ID NOs: 186, 188, 190 and 192, respectively;
(ix) further comprises heavy chain variable domain FRs 1, 2, 3 and 4 comprising amino acids SEQ ID NOs: 200, 202, 204 and 206, respectively;
(x) further comprises heavy chain variable domain FRs 1, 2, 3 and 4 comprising amino acids SEQ ID NOs: 214, 216, 218 and 220, respectively;
(xi) further comprises heavy chain variable domain FRs 1, 2, 3 and 4 comprising amino acids SEQ ID NOs: 228, 230, 232 and 234, respectively; or
(xii) further comprises heavy chain variable domain FRs 1, 2, 3 and 4 comprising amino acids SEQ ID NOs: 242, 244, 246 and 248, respectively.

3. The isolated antibody or the fragment thereof of claim 1, wherein said antibody of
(i) further comprises light chain variable domain framework regions (FRs) 1, 2, 3 and 4 comprising amino acids SEQ ID NOs: 29, 31, 33 and 35, respectively;
(ii) further comprises light chain variable domain FRs 1, 2, 3 and 4 comprising amino acids SEQ ID NOs: 43, 45, 47 and 49, respectively;
(iii) further comprises light chain variable domain FRs 1, 2, 3 and 4 comprising amino acids SEQ ID NOs: 123, 125, 127 and 129, respectively;
(iv) further comprises light chain variable domain FRs 1, 2, 3 and 4 comprising amino acids SEQ ID NOs: 138, 140, 142 and 144, respectively;
(v) further comprises light chain variable domain FRs 1, 2, 3 and 4 comprising amino acids SEQ ID NOs: 153, 155, 157 and 159, respectively;
(vi) further comprises light chain variable domain FRs 1, 2, 3 and 4 comprising amino acids SEQ ID NOs: 165, 167, 169 and 171, respectively;
(vii) further comprises light chain variable domain FRs 1, 2, 3 and 4 comprising amino acids SEQ ID NOs: 179, 181, 183, 185, respectively;
(viii) further comprises light chain variable domain FRs 1, 2, 3 and 4 comprising amino acids SEQ ID NOs: 193, 195, 197 and 199, respectively;
(ix) further comprises light chain variable domain FRs 1, 2, 3 and 4 comprising amino acids SEQ ID NOs: 207, 209, 211 and 213, respectively;
(x) further comprises light chain variable domain FRs 1, 2, 3 and 4 comprising amino acids SEQ ID NOs: 221, 223, 225 and 227, respectively;
(xi) further comprises light chain variable domain FRs 1, 2, 3 and 4 comprising amino acids SEQ ID NOs: 235, 237, 239 and 241, respectively; or
(xii) further comprises light chain variable domain FRs 1, 2, 3 and 4 comprising amino acids SEQ ID NOs: 249, 251, 253 and 255, respectively.

4. The isolated antibody or the fragment thereof of claim 1, wherein said antibody comprises a heavy chain variable domain comprising an amino acid sequence at least 90% identical to the amino acid sequence selected from the group consisting of SEQ ID NOs: 92, 96, 256, 258, 260, 262, 264, 266, 268, 270, 272 and 274.

5. The isolated antibody or the fragment thereof of claim 1, wherein said antibody comprises a heavy chain variable domain comprising the amino acid sequence selected from the group consisting of SEQ ID NOs: 92, 96, 256, 258, 260, 262, 264, 266, 268, 270, 272 and 274.

6. The isolated antibody or the fragment thereof of claim 1, wherein said antibody comprises a light chain variable domain comprising an amino acid sequence at least 90% identical to the amino acid sequence selected from the group consisting of SEQ ID NOs: 93, 97, 257, 259, 261, 263, 265, 267, 269, 271, 273 and 275.

7. The isolated antibody or the fragment thereof of claim 1, wherein said antibody comprises a light chain variable domain comprising the amino acid sequence selected from the group consisting of SEQ ID NOs: 93, 97, 257, 259, 261, 263, 265, 267, 269, 271, 273 and 275.

8. The isolated antibody or the fragment thereof of claim 1, being a single chain Fv (scFv).

9. The isolated antibody or the fragment thereof of claim 8, wherein said scFv is selected from the group consisting of SEQ ID NOs: 110, 111 and 100-109.

10. The isolated antibody or the fragment thereof of claim 1, being conjugated to a functional moiety.

11. A chimeric antigen receptor (CAR) comprising the p32 antibody fragment of claim 1 and an intracellular signaling domain, said intracellular signaling domain comprising a co-stimulatory signaling region and an activation domain.

12. The CAR of claim 11, wherein said co-stimulatory signaling region comprises CD28, 41BB, OX40, ICOS, CD27, MYD88-CD40, Toll-like receptor (TLRs) or KIR2DS2; and/or
wherein said activation domain comprises FcRgamma or CD3zeta.

13. The CAR of claim 11, comprising the amino acid sequence as set forth by SEQ ID NO: 88 or 90.

14. A chimeric antigen receptor (CAR) comprising a p32 antibody fragment and an intracellular signaling domain, said intracellular signaling domain comprising a co-stimulatory signaling region and an activation domain, wherein said co-stimulatory signaling region comprises the 41BB amino acid sequence and said activation domain comprises the CD3zeta amino acid sequence.

15. The CAR of claim 14, wherein said p32 antibody comprises a heavy chain CDRs 1, 2 and 3 as set forth by SEQ ID NOs: 9, 11, and 13, respectively and/or wherein said p32 antibody comprises light chain CDRs 1, 2 and 3 as set forth by SEQ ID NOs: 16, 18 and 20, respectively and/or wherein said p32 antibody comprises heavy chain variable domain comprising framework regions (FRs) 1, 2, 3 and 4 as set forth by SEQ ID NOs: 8, 10, 12, and 14, respectively.

16. The CAR of claim 14, further comprises light chain variable domain comprising framework regions (FRs) 1, 2, 3 and 4 as set forth by SEQ ID NOs: 15, 17, 19 and 21, respectively.

17. An isolated T lymphocyte cell (T cell) transduced to express the CAR of 11.
